SEQUENCE LISTING (36 sequences)

SEQ ID NO: 1 - DNA, 200 bp, synthetic construct
  misc_feature 1..200: note = synthetic polynucleotide
  source 1..200: mol_type = other DNA
agctctcctaccactagactgctgagacccgctgctctgctcaggactcgatttccagtacacaatctccctctttgaaaagtaccacacatcctggggtgctcttgcatttgtgtgacactttgctagccaggctcagtcctgggttccaggtggggactcaaacacactggcacgagtctacattggatattcttggt

SEQ ID NO: 2 - DNA, 199 bp, synthetic construct
  misc_feature 1..199: note = synthetic polynucleotide
  source 1..199: mol_type = other DNA
gctccccattcctcactggcccagcccctcttccctactctttctagcccctgcctcatctccctggctgccattgggagcctgccccactggaagccagtcgagataacttcgtataatgtatgctatacgaagttatatgcatggcctccgcgccgggttttggcgcctcccgcgggcgcccccctcctcacggcga

SEQ ID NO: 3 - DNA, 200 bp, synthetic construct
  misc_feature 1..200: note = synthetic polynucleotide
  source 1..200: mol_type = other DNA
cattctcagtattgttttgccaagttctaattccatcagacctcgacctgcagcccctagataacttcgtataatgtatgctatacgaagttatgctagctgtctcatagaggctggcgatctggctcagggacagccagtactgcaaagagtatccttgttcataccttctcctagtggccatctccctgggacagtca

SEQ ID NO: 4 - DNA, 208 bp, synthetic construct
  misc_feature 1..208: note = synthetic polynucleotide
  source 1..208: mol_type = other DNA
atccatttagcctttctctgatcactaagttggacagttggacagtcttcctcaaattagcttagactatcaaaattatactgtatttttggtatttccagcgatcgcttcagttacaaggctgttgaatgcacagaagcaaggataacactgattttttcactggtcagaataaaaattattgattgctcttttgcttatagtattc

SEQ ID NO: 5 - DNA, 2028 bp, synthetic construct
  misc_feature 1..2028: note = synthetic polynucleotide
  source 1..2028: mol_type = other DNA
aatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcatataatgtccatcagtaaatcttggtggtggtggcaataataaacttctactgataggtagaatggtgtgcaagcttgtccaatcacggattgcaggccacatgcggcccaggacaactttgaatgtggcccaacacaaattcataaactttcatacatctcgtttttagctcatcagctatcattagcggtagtgtatttaaagtgtggcccaagacaattcttcttattccaatgtggcccagggaaatcaaaagattggatgcccctggtatagaaaactaatagtgacagtgttcatatttcatgctttcccaaatacaggtattttattttcacattctttttgccatgtttatataataataaagaaaaaccctgttgatttgttggagccattgttatctgacagaaaataattgtttatattttttgcactacactgtctaaaattagcaagctctcttctaatggaactgtaagaaagatgaaatatttttgttttattataaatttatttcaccttaattctggtaatactcactgagtgactgtggggtgggaaatgatctcttaagaatttgatttctttctattccatagtacaaactcgttctctgttgaaacattcttctatcaccccagtgccctatccatgtacatgtgttcttattgctctagtcaaacggtgcttataaatatctttcagaaagtttaggagaaatctgtatcctatttgacttccaataatcatgtattggctgtcagcttcttacctactctcagtccagagaaatagtatttggcagccactctttaaagtttatgggttgtggattgtggcggttgatttattttttttatttcaattgggatagaattttttaatatacctgtatttttgttttgttttatgtagcttttctattagggagagtaggaaaagtgcaccattttcttctctaaatttccagtccagtctttaggggaatgttagtcttcctgagatgggggaaggaaaatcataatgccagtcactttgcaaataatattttatagtgataaatggttcattttggttacataggcatacaagtgggcttaaaacttggaatttaccagggctcaaaattaaaattcttacattagttactcgatatggatcgcttcagttgatcttagaaaactcaaggcatagatctgcaacctcgagataacttcgtataatgtatgctatacgaagttatatgcatggcctccgcgccgggttttggcgcctcccgcgggcgcccccctcctcacggcg

SEQ ID NO: 6 - DNA, 200 bp, synthetic construct
  misc_feature 1..200: note = synthetic polynucleotide
  source 1..200: mol_type = other DNA
cattctcagtattgttttgccaagttctaattccatcagacctcgacctgcagcccctagataacttcgtataatgtatgctatacgaagttatgctagcgtgatagtccttcacggaaagtacaagaatacacagaaaactgctgtttacattagtctttcacgtttttattttattctcacaaattttaatgcaatac

SEQ ID NO: 7 - DNA, 20 bp, synthetic construct
  misc_feature 1..20: note = synthetic oligonucleotide
  source 1..20: mol_type = other DNA
agggatttgaatcacgtttg

SEQ ID NO: 8 - DNA, 20 bp, synthetic construct
  misc_feature 1..20: note = synthetic oligonucleotide
  source 1..20: mol_type = other DNA
tttactggcaacatcaacag

SEQ ID NO: 9 - DNA, 20 bp, synthetic construct
  misc_feature 1..20: note = synthetic oligonucleotide
  source 1..20: mol_type = other DNA
gcccagggaaatcaaaagat

SEQ ID NO: 10 - DNA, 20 bp, synthetic construct
  misc_feature 1..20: note = synthetic oligonucleotide
  source 1..20: mol_type = other DNA
tggctccaacaaatcaacag

SEQ ID NO: 11 - DNA, 4201 bp, Homo sapiens
  source 1..4201: mol_type = other DNA
tccggcccgcacccacccccaagaggggccttcagctttggggctcagaggcacgacctcctggggagggttaaaaggcagacgcccccccgccccccgcgcccccgcgccccgactccttcgccgcctccagcctctcgccagtgggaagcggggagcagccgcgcggccggagtccggaggcgaggggaggtcggccgcaacttccccggtccaccttaagaggacgatgtagccagctcgcagcgctgaccttagaaaaacaagtttgcgcaaagtggagcggggacccggcctctgggcagccccggcggcgcttccagtgccttccagccctcgcgggcggcgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatga

SEQ ID NO: 12 - protein, 504 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 13 - DNA, 4109 bp, Homo sapiens
  source 1..4109: mol_type = other DNA
ctctctggccgcccctggctttatttctcgcgcgcttggggtctctcccagtctccgtctctccatttctcctggggggcggggagggggggtctccaaaaaccgcggcggcggcggcggccgctccaggcgcccgttccggagtcggggggaggcccagccgggaggggggaagggggggagccttagtcatttccccgctccagcctgctcccgcccgagcgcgcactcacggccgctctccctcctcgctccgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatga

SEQ ID NO: 14 - DNA, 3868 bp, Homo sapiens
  source 1..3868: mol_type = other DNA
cgctcgctcgcagagaagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatga

SEQ ID NO: 15 - DNA, 4031 bp, Mus musculus
  source 1..4031: mol_type = other DNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagtttcacccaccgtgaaggtcacccaacagtccccgacgtcaatgaaccaggtgaacctcacctgccgggctgagaggttctaccccgaggatctccagctgatctggctggagaatggaaacgtatcacggaatgacacgcccaagaatctcacaaagaacacggatgggacctataattacacaagcttgttcctggtgaactcatctgctcatagagaggacgtggtgttcacgtgccaggtgaagcacgaccaacagccagcgatcacccgaaaccataccgtgctgggatttgcccactcgagtgatcaagggagcatgcaaaccttccctgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccagatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 16 - protein, 509 aa, Mus musculus
MEPAGPAPGRLGPLLLCLLLSASCFCTGATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLAKPSPPEVSGPADRGIPDQKVNFTCKSHGFSPRNITLKWFKDGQELHPLETTVNPSGKNVSYNISSTVRVVLNSMDVNSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGFAHSSDQGSMQTFPDNNATHNWNVFIGVGVACALLVVLLMAALYLLRIKQKKAKGSTSSTRLHEPEKNAREITQIQDTNDINDITYADLNLPKEKKPAPRAPEPNNHTEYASIETGKVPRPEDTLTYADLDMVHLSRAQPAPKPEPSFSEYASVQVQRK

SEQ ID NO: 17 - DNA, 3377 bp, Mus musculus
  source 1..3377: mol_type = other DNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccagatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 18 - protein, 291 aa, Mus musculus
MEPAGPAPGRLGPLLLCLLLSASCFCTGATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLDNNATHNWNVFIGVGVACALLVVLLMAALYLLRIKQKKAKGSTSSTRLHEPEKNAREITQIQDTNDINDITYADLNLPKEKKPAPRAPEPNNHTEYASIETGKVPRPEDTLTYADLDMVHLSRAQPAPKPEPSFSEYASVQVQRK

SEQ ID NO: 19 - DNA, 4043 bp, Mus musculus
  source 1..4043: mol_type = other DNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagtttcacccaccgtgaaggtcacccaacagtccccgacgtcaatgaaccaggtgaacctcacctgccgggctgagaggttctaccccgaggatctccagctgatctggctggagaatggaaacgtatcacggaatgacacgcccaagaatctcacaaagaacacggatgggacctataattacacaagcttgttcctggtgaactcatctgctcatagagaggacgtggtgttcacgtgccaggtgaagcacgaccaacagccagcgatcacccgaaaccataccgtgctgggatttgcccactcgagtgatcaagggagcatgcaaaccttccctgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccaggtacagtctttgatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 20 - protein, 513 aa, Mus musculus
MEPAGPAPGRLGPLLLCLLLSASCFCTGATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLAKPSPPEVSGPADRGIPDQKVNFTCKSHGFSPRNITLKWFKDGQELHPLETTVNPSGKNVSYNISSTVRVVLNSMDVNSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGFAHSSDQGSMQTFPDNNATHNWNVFIGVGVACALLVVLLMAALYLLRIKQKKAKGSTSSTRLHEPEKNAREITQVQSLIQDTNDINDITYADLNLPKEKKPAPRAPEPNNHTEYASIETGKVPRPEDTLTYADLDMVHLSRAQPAPKPEPSFSEYASVQVQRK

SEQ ID NO: 21 - DNA, 3845 bp, Mus musculus
  source 1..3845: mol_type = other DNA
aagctcccctgccgcgggcagcctcttgcccactggagtctaaggactggccgggtgagaggccgagaccagggggcgatcggccgccacttccccagtccaccttaagaggaccaagtagccagcccgccgcgccgacctcagaaaaacaagtttgcgcaaagtggtgcgcggccagcctctgggcagagggagcggtgcttccaccgcctggcagccctgcgcgcggcggcgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagtttcacccaccgtgaaggtcacccaacagtccccgacgtcaatgaaccaggtgaacctcacctgccgggctgagaggttctaccccgaggatctccagctgatctggctggagaatggaaacgtatcacggaatgacacgcccaagaatctcacaaagaacacggatgggacctataattacacaagcttgttcctggtgaactcatctgctcatagagaggacgtggtgttcacgtgccaggtgaagcacgaccaacagccagcgatcacccgaaaccataccgtgctgggatttgcccactcgagtgatcaagggagcatgcaaaccttccctgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccaggtacagtctttgatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 22 - DNA, 3389 bp, Mus musculus
  source 1..3389: mol_type = other DNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccaggtacagtctttgatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 23 - protein, 295 aa, Mus musculus
MEPAGPAPGRLGPLLLCLLLSASCFCTGATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLDNNATHNWNVFIGVGVACALLVVLLMAALYLLRIKQKKAKGSTSSTRLHEPEKNAREITQVQSLIQDTNDINDITYADLNLPKEKKPAPRAPEPNNHTEYASIETGKVPRPEDTLTYADLDMVHLSRAQPAPKPEPSFSEYASVQVQRK

SEQ ID NO: 24 - DNA, 3020 bp, Mus musculus
  source 1..3020: mol_type = other DNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacagataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccagatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 25 - protein, 172 aa, Mus musculus
MEPAGPAPGRLGPLLLCLLLSASCFCTDNNATHNWNVFIGVGVACALLVVLLMAALYLLRIKQKKAKGSTSSTRLHEPEKNAREITQIQDTNDINDITYADLNLPKEKKPAPRAPEPNNHTEYASIETGKVPRPEDTLTYADLDMVHLSRAQPAPKPEPSFSEYASVQVQRK

SEQ ID NO: 26 - DNA, 3393 bp, Mus musculus
  source 1..3393: mol_type = other DNA
actctaccctgcagccttcagcttggcacaaactaaacagtgactcttccccaagtgccgagtttaattcctggcttggccgaaaggattcagagagggataagcagcccctctggccttcagtgccaaaataagaaagagtatttcacatccacaagcaggaagtacacttcatacctctctaagataaaagacctattcacaatcaaaaatgtccctgcagatggtaacagtgggtcataacatagccttaattcaaccaggcttctcacttatgaattttgatggccaagttttcttctttggccagaaaggctggcctaagagatcctgtcctactggagtctttcattttgatataaaacaaaatcatctcaaactgaagcctgcaatcttctctaaagattcctgctacctcccacctcttcgttatccagctacttgctcatacaaaggcagcatagactctgacaagcatcaatatatcattcacggagggaaaacaccaaacaatgagctttccgataagatttatatcatgtctgtcgcttgcaagaataacaaaaaagttactttccgttgcacagagaaagacttagtaggagatgtccctgaacccagatacggccattccattgacgtggtgtatagtcgagggaaaagcatgggtgttctctttggaggacgttcatacatgccttctacccagagaaccacagaaaaatggaatagtgtagctgactgcctaccccatgttttcttgatagattttgaatttgggtgtgctacatcatatattctcccagaacttcaggatgggctgtcttttcatgtttctattgccagaaacgataccgtttatattttgggaggacactcacttgccagtaatatacgccctgctaacttgtatagaataagagtggaccttcccctgggtaccccagcagtgaattgcacagtcttgccaggaggaatctctgtctccagtgcaatcctcactcaaacaaacaatgatgaatttgttattgtgggtggttatcagctggaaaatcagaaaaggatggtctgcagccttgtctctctaggggacaacacgattgaaatcagtgagatggagactcctgactggacctcagatattaagcatagcaaaatatggtttggaagcaacatgggaaacgggactattttccttggcataccaggagacaataagcaggctatgtcagaagcattctatttctatactttgagatgctctgaagaggatttgagtgaagatcagaaaattgtctccaacagtcagacatcaacagaagatcctggggactccactccctttgaagactcagaggaattttgtttcagtgctgaagcaaccagttttgatggtgacgatgaatttgacacctacaatgaagatgatgaagatgacgagtctgtaaccggctactggataacatgttgccctacttgtgatgttgacatcaatacctgggttccgttctattcaacggagctcaataaacccgccatgatctattgttctcatggggatgggcactgggtacatgcccagtgcatggatttggaagaacgcacactcatccacttgtcagaaggaagcaacaagtattattgcaatgaacatgtacagatagcaagagcattgcaaactcccaaaagaaacccccccttacaaaaacctccaatgaaatccctccacaaaaaaggctctgggaaagtcttgactcctgccaagaaatccttccttagaagactgtttgattaatttagcaaaagcccctcagactcaggtatattgctctctgaatctactttcaatcataaacattattttgatttttgtttactgaaatctctatgttatgttttagttatgtgaattaagtgctgttgtgatttattgttaagtataactattctaatgtgtgttttttaacatcttatccaggaatgtcttaaatgagaaatgttatacagttttccattaaggatatcagtgataaagtatagaactcttacattattttgtaacaatctacatattgaatagtaactaaataccaataaataaactaatgcacaaaaagttaagttcttttgtgtaataagtagcctatagttggtttaaacagttaaaaccaacagctatatcccacactactgctgtttataaattttaaggtggcctctggtttatacttatgagcagaattatatatattggtcaataccatgaagaaaaatttaattctatatcaagccaggcatggtgatggtgatacatgcctgtaatcctggcacttaggaagtggaagaaggaagtttgtgagtttgatgcttgttgaggtatgaccttttgctatgtattgtagtgtatgagccccaagacctgcttgacccagagacaagagagtccacacatagatccaagtaatgctatgtgaccttgccccccggttacttgtgattaggtgaataaagatgtcaacagccaatagctgggcagaagagccaaaagtggggattgagggtaccctggcttgatgtaggaggagaccatgaggaaaggggagaaaaaagtgatggaggaggagaaagatgccatgagctaggagttaagaaagcatggccatgagtgctggccaattggagttaagagcagcccagatgaaacatagtaagtaataactcagggttatcgatagaaaatagattttagtgccgtactctccccagccctagagctgactatggcttactgtaaatataaagtttgtatgtgtcttttatccaggaactaaatggtcaaaggtggagtagaaactctggattgggattaaatttttctacaacaaatgctggcctgggctagattttatctcatatccgaaggctgacagaacacagagcactggtaacattgccacctgccatgcacaaagacctgagtctaatactgtggacattttcttgaagtatctacatgtacttctggagtgaaaacatattccaacaatatgcctttgtttaaatcactcactcactttgggccctcacattatatcctttcaaaatcaatggttcacccctttgaaaatgcttagccatagtccctcatcttccttaaagacagttgtcatctctggaaatagtcacatgtcattcaaggtccaatactgtgcagctctgaagtatggcattaccactttaagtgaaaagtgaaatatgaacatgagctcagacaaaggtttgggactatcactctcaaggaggctctactgctaagtcctgaactgctttcacatgaatacagaaattataacaaaaaatatgtaatcaataaaaagaaaactttcatattcc

SEQ ID NO: 27 - protein, 527 aa, Mus musculus
MSLQMVTVGHNIALIQPGFSLMNFDGQVFFFGQKGWPKRSCPTGVFHFDIKQNHLKLKPAIFSKDSCYLPPLRYPATCSYKGSIDSDKHQYIIHGGKTPNNELSDKIYIMSVACKNNKKVTFRCTEKDLVGDVPEPRYGHSIDVVYSRGKSMGVLFGGRSYMPSTQRTTEKWNSVADCLPHVFLIDFEFGCATSYILPELQDGLSFHVSIARNDTVYILGGHSLASNIRPANLYRIRVDLPLGTPAVNCTVLPGGISVSSAILTQTNNDEFVIVGGYQLENQKRMVCSLVSLGDNTIEISEMETPDWTSDIKHSKIWFGSNMGNGTIFLGIPGDNKQAMSEAFYFYTLRCSEEDLSEDQKIVSNSQTSTEDPGDSTPFEDSEEFCFSAEATSFDGDDEFDTYNEDDEDDESVTGYWITCCPTCDVDINTWVPFYSTELNKPAMIYCSHGDGHWVHAQCMDLEERTLIHLSEGSNKYYCNEHVQIARALQTPKRNPPLQKPPMKSLHKKGSGKVLTPAKKSFLRRLFD

SEQ ID NO: 28 - DNA, 1612 bp, Mus musculus
  source 1..1612: mol_type = other DNA
gacacagactacacccagagaaagaagagcaagcaccatgttgaaactattattgtcacctagatccttcttagtccttcagctgctcctgctgagggcagggtggagctccaaggtcctcatgtccagtgcgaatgaagacatcaaagctgatttgatcctgacttctacagcccctgaacacctcagtgctcctactctgccccttccagaggttcagtgctttgtgttcaacatagagtacatgaattgcacttggaatagcagttctgagcctcaggcaaccaacctcacgctgcactataggtacaaggtatctgataataatacattccaggagtgcagtcactatttgttctccaaagagattacttctggctgtcagatacaaaaagaagatatccagctctaccagacatttgttgtccagctccaggacccccagaaaccccagaggcgagctgtacagaagctaaacctacagaatcttgtgatcccacgggctccagaaaatctaacactcagcaatctgagtgaatcccagctagagctgagatggaaaagcagacatattaaagaacgctgtttacaatacttggtgcagtaccggagcaacagagatcgaagctggacggaactaatagtgaatcatgaacctagattctccctgcctagtgtggatgagctgaaacggtacacatttcgggttcggagccgctataacccaatctgtggaagttctcaacagtggagtaaatggagccagcctgtccactgggggagtcatactgtagaggagaatccttccttgtttgcactggaagctgtgcttatccctgttggcaccatggggttgattattaccctgatctttgtgtactgttggttggaacgaatgcctccaattccccccatcaagaatctagaggatctggttactgaataccaagggaacttttcggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcctgaacatcaatcctttgatggaacctcaaagtcctatagtcctaagtgacgctaacctcccctactcaccttggcaatctggatccaatgctcactgccttcccttggggctaagtttcgatttcctgtcccatgtaactgcttttctgttccatatgccctacttgagagtgtcccttgccctctttccctgcacaagccctcccatgcccagcctaacacctttccactttctttgaagagagtcttaccctgtagcccagggtggctgggagctcactatgtaggccaggttggcctccaactcacaggctatcctcccacctctgcctcataagagttggggttactggcatgcaccaccacacccagcatggtccttctcttttataggattctccctccctttttctacctatgattcaactgtttccaaatcaacaagaaataaagtttttaaccaatgat

SEQ ID NO: 29 - protein, 369 aa, Mus musculus
MLKLLLSPRSFLVLQLLLLRAGWSSKVLMSSANEDIKADLILTSTAPEHLSAPTLPLPEVQCFVFNIEYMNCTWNSSSEPQATNLTLHYRYKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEENPSLFALEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 30 - DNA, 2012 bp, Homo sapiens
  source 1..2012: mol_type = other DNA
gttgggactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgacgtcacatggagcacagaaatcaatgttagcagatagccagcccatacaagatcgtattgtattgtaggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcatataatgtccatcagtaaatcttggtggtggtggcaataataaacttctactgataggtagaatggtgtgcaagcttgtccaatcacggattgcaggccacatgcggcccaggacaactttgaatgtggcccaacacaaattcataaactttcatacatctcgtttttagctcatcagctatcattagcggtagtgtatttaaagtgtggcccaagacaattcttcttattccaatgtggcccagggaaatcaaaagattggatgcccctggtatagaaaactaatagtgacagtgttcatatttcatgctttcccaaatacaggtattttattttcacattctttttgccatgtttatataataataaagaaaaaccctgttgatttgttggagccattgttatctgacagaaaataattgtttatattttttgcactacactgtctaaaattagcaagctctcttctaatggaactgtaagaaagatgaaatatttttgttttattataaatttatttcaccttaaaaaaaaaaaa

SEQ ID NO: 31 - protein, 160 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIN

SEQ ID NO: 32 - DNA, 2333 bp, Homo sapiens
  source 1..2333: mol_type = other DNA
gttgggactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgacgtcacatggagcacagaaatcaatgttagcagatagccagcccatacaagatcgttttcaactagtggccccactgtgtccggaattgatgggttcttggtctcactgacttcaagaatgaagccgcggaccctcgcggtgagtgttacagctcttaaggtggcgcatctggagtttgttccttctgatgttcggatgtgttcggagtttcttccttctggtgggttcgtggtctcgctggctcaggagtgaagctacagaccttcgcggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggatgcagctaatatacccagttggcccaaagcacctaacctatagttatataatctgactctcagttcagttttactctactaatgccttcatggtattgggaaccatagatttgtgcagctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcatataatgtccatcagtaaatcttggtggtggtggcaataataaacttctactgataggtagaatggtgtgcaagcttgtccaatcacggattgcaggccacatgcggcccaggacaactttgaatgtggcccaacacaaattcataaactttcatacatctcgtttttagctcatcagctatcattagcggtagtgtatttaaagtgtggcccaagacaattcttcttattccaatgtggcccagggaaatcaaaagattggatgcccctggtatagaaaactaatagtgacagtgttcatatttcatgctttcccaaatacaggtattttattttcacattctttttgccatgtttatataataataaagaaaaaccctgttgatttgttggagccattgttatctgacagaaaataattgtttatattttttgcactacactgtctaaaattagcaagctctcttctaatggaactgtaagaaagatgaaatatttttgttttattataaatttatttcaccttaaaaaaaaaaaa

SEQ ID NO: 33 - protein, 135 aa, Mus musculus
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 34 - DNA, 1297 bp, Mus musculus
  source 1..1297: mol_type = other DNA
ttcttgaccaagacttcaatactcagtggcactgtattccccttctgtccagccactcttccccagagttctcttcttcatcctcccccttgcagagtagggcagcttgcaggtcctcctgcaagtctctcccaattctctgcgcccaaaagacttgcagtgcatctccttacgcgctgcagggaccttgccagggcaggactgcccccgcccagttgcagagttggacgaagacgggatcctgctgtgtttggaaggctgagttccacatctaacagctcagagaggtcaggaaagaatccaccttgacacatggccctctggctcttcaaagcactgcctcttcatggtccttgctggtgaggtccttaagaacacagaaacccatgtcagcagataaccagcctacaggaggccaagaagagttctggatggatggcagctggaagcccatcgccatagccagctcatcttcaacattgaagctcttacctgggcattaagtaatgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcctgactgcatgcgagcctcttccgtgtttctgttattaaggtacctccacctgctgctcagaggcagcacagctccatgcatttgaaatctgctgggcaaactaagcttcctaacaaggagataatgagccacttggatcacatgaaatcttggaaatgaagagaggaaaagagctcgtctcagacttatttttgcttgcttatttttaatttattgcttcatttgtacatatttgtaatataacagaagatgtggaataaagttgtatggatattttatcaattgaaatttaaaaaaaaaaaaaa

SEQ ID NO: 35 - protein, 160 aa, Mus musculus
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFIN

SEQ ID NO: 36 - DNA, 1287 bp, Mus musculus
  source 1..1287: mol_type = other DNA
ttcttgaccaagacttcaatactcagtggcactgtattccccttctgtccagccactcttccccagagttctcttcttcatcctcccccttgcagagtagggcagcttgcaggtcctcctgcaagtctctcccaattctctgcgcccaaaagacttgcagtgcatctccttacgcgctgcagggaccttgccagggcaggactgcccccgcccagttgcagagttggacgaagacgggatcctgctgtgtttggaaggctgagttccacatctaacagctcagagagaatccaccttgacacatggccctctggctcttcaaagcactgcctcttcatggtccttgctggtgaggtccttaagaacacagaaacccatgtcagcagataaccagcctacaggaggccaagaagagttctggatggatggcagctggaagcccatcgccatagccagctcatcttcaacattgaagctcttacctgggcattaagtaatgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcctgactgcatgcgagcctcttccgtgtttctgttattaaggtacctccacctgctgctcagaggcagcacagctccatgcatttgaaatctgctgggcaaactaagcttcctaacaaggagataatgagccacttggatcacatgaaatcttggaaatgaagagaggaaaagagctcgtctcagacttatttttgcttgcttatttttaatttattgcttcatttgtacatatttgtaatataacagaagatgtggaataaagttgtatggatattttatcaattgaaatttaaaaaaaaaaaaaa

(19) United States
(12) Patent Application Publication
(10) Pub. No.: US 2026/0305588 A1
(43) Pub. Date: Oct. 8, 2026

(54) Genetically Modified Non-Human Animals and Methods of Use Thereof

(71) Applicants: Regeneron Pharmaceuticals, Inc., Tarrytown, NY (US); Yale University, New Haven, CT (US); Institute for Research in Biomedicine (IRB), Bellinzona (CH)

(72) Inventors: Dietmar Herndler-Brandstetter, New Haven, CT (US); Richard A. Flavell, Guilford, CT (US); Davor Frleta, Tarrytown, NY (US); Cagan Gurer, Tarrytown, NY (US); Markus Gabriel Manz, Zurich (CH); Andrew J. Murphy, Tarrytown, NY (US); Noah W. Palm, New Haven, CT (US); Liang Shan, New Haven, CT (US); Sean Stevens, Del Mar, CA (US); Till Strowig, Braunschweig (DE); George D. Yancopoulos, Yorktown Heights, NY (US); Marcel de Zoete, Amersfoort (NL)

(21) Appl. No.: 19/640,275

(22) Filed: Apr. 6, 2026

Related U.S. Application Data
(63) Continuation of application No. 18/152,086, filed on Jan. 9, 2023, now Pat. No. 12,672,643.
Application No. 18/152,086 is a continuation of application No. 16/782,708, filed on Feb. 5, 2020, now Pat. No. 11,576,356.
Application No. 16/782,708 is a continuation of application No. 15/954,450, filed on Apr. 16, 2018, now Pat. No. 10,561,126.
Application No. 15/954,450 is a continuation of application No. 15/097,239, filed on Apr. 12, 2016, now Pat. No. 10,123,518.
(60) Provisional application No. 62/146,938, filed on Apr. 13, 2015.
(60) Provisional application No. 62/148,667, filed on Apr. 16, 2015.
(60) Provisional application No. 62/287,842, filed on Jan. 27, 2016.

(51) Int. Cl. A01K 67/0278; A01K 67/0271; A61K 49/00

(52) U.S. Cl. CPC A01K 67/0278; A01K 67/0271; A61K 49/0008; A01K 2207/12; A01K 2217/072; A01K 2217/15; A01K 2227/105; A01K 2267/0331; A01K 2267/0337

(57) ABSTRACT

Genetically modified non-human animals expressing human SIRPα and human IL-15 from the non-human animal genome are provided. Also provided are methods for making non-human animals expressing human SIRPα and human IL-15 from the non-human animal genome, and methods for using non-human animals expressing human SIRPα and human IL-15 from the non-human animal genome. These animals and methods find many uses in the art, including, for example, in modeling human T cell and/or natural killer (NK) cell development and function, in modeling human pathogen infection of human T cells and/or NK cells, and in various in vivo screens.

### CROSS-REFERENCE

This application is a continuation of U.S. application Ser. No. 18/152,086, filed Jan. 9, 2023, which application is a continuation of U.S. application Ser. No. 16/782,708, filed Feb. 5, 2020, now U.S. Pat. No. 11,576,356, which application is a continuation of U.S. application Ser. No. 15/954,450, filed Apr. 16, 2018, now U.S. Pat. No. 10,561,126, which application is a continuation of U.S. application Ser. No. 15/097,239, filed Apr. 12, 2016, now U.S. Pat. No. 10,123,518, which application claims the benefit of U.S. Provisional Application Nos. 62/146,938, filed Apr. 13, 2015; 62/148,667, filed Apr. 16, 2015; and 62/287,842, filed Jan. 27, 2016, the disclosure of each of which is incorporated herein by reference in its entirety.

### INCORPORATION BY REFERENCE OF SEQUENCE LISTING PROVIDED AS A SEQUENCE LISTING XML FILE

A Sequence Listing is provided herewith as a Sequence Listing XML, REGN-016CON3_SEQ_LIST, created on Apr. 20, 2023, and having a size of 88,390 bytes. The contents of the Sequence Listing XML are incorporated herein by reference in their entirety.

### FIELD OF INVENTION

The invention relates to the field of genetically modified non-human animals.

### INTRODUCTION

Genetically modified non-human animals, such as humanized mice, hold great promise for translational research, as they allow modeling and studying of human diseases in vivo. Within the last decade, considerable progress has been made in developing humanized mice by genetically inserting human genes that are essential for the proper development and function of human immune cells in the mouse. However, some limitations still restrict the utility of humanized mice in translational research. In particular, the development and survival of human T cells is suboptimal.

Although the bone marrow-liver-thymus (BLT) model has been shown to improve intestinal T cell reconstitution in NS/NSG-BLT mice (Denton P W, Nochi T, Lim A et al. Mucosal Immunol 2012; 5:555-566, Nochi T, Denton P W, Wahl A et al. Cell Rep 2013; 3:1874-1884), those mice have been shown to develop graft-versus-host disease, resulting in massive immune cell infiltration in multiple tissues (Greenblatt M B, Vrbanac V, Tivey T et al. PLOS One 2012; 7:e44664). Therefore, current humanized mouse models still lack proper development and function of human T cells. In particular, the absence of human tissue-resident memory T cells prevents the use of humanized mice as a preclinical tool to develop and test more efficient immunization strategies that aim to induce long-lasting mucosal immunity against pathogens such as HIV.

In order to better understand the development and survival of human tissue-resident T cells and provide a model to test novel immunization strategies to induce long-lasting T cell-dependent mucosal immunity, it would be useful to have a genetically modified non-human animal which develops human tissue-resident T cells. Such a mouse model could also be used to study the interaction of human tissue-resident immune cells with the gut microbiota, for example, how the microbiota may shape the development and survival of human immune cells in the small intestine and colon.

In addition, there is a need in the art for non-human animal models of human Natural Killer (NK) cell development and function.

### SUMMARY

Genetically modified non-human animals expressing human SIRPα and human IL-15 from the non-human animal genome are provided. Also provided are methods for making non-human animals expressing human SIRPα and human IL-15 from the non-human animal genome, and methods for using non-human animals expressing human SIRPα and human IL-15 from the non-human animal genome. These animals and methods find many uses in the art, including, for example, in modeling human T cell and/or natural killer (NK) cell development and function; in modeling human pathogen infection of human T cells and/or NK cells; in in vivo screens for agents that inhibit infection by a pathogen that activates, induces and/or targets T cells and/or NK cells; in in vivo screens for agents that modulate the development and/or function of human T cells and/or NK cells, e.g. in a healthy or a diseased state; in in vivo screens for agents that are toxic to human T cells and/or NK cells; in in vivo screens for agents that prevent against, mitigate, or reverse the toxic effects of toxic agents on human T cells and/or NK cells; in in vivo screens of candidate T cell-inducing vaccines; and in in vivo and in vitro screens for agents that inhibit tumor growth and/or infection by activating NK cell-mediated antibody dependent cellular cytotoxicity (ADCC) processes.

In a first aspect, the present disclosure provides a genetically modified non-human animal, including: a nucleic acid sequence incorporated into the genome of the genetically modified non-human animal, which sequence encodes a human SIRPα protein and is operably linked to a SIRPα gene promoter; and a nucleic acid sequence incorporated into the genome of the genetically modified non-human animal, which sequence encodes a human IL-15 protein and is operably linked to an IL-15 gene promoter, wherein the genetically modified non-human animal expresses the human SIRPα protein and the human IL-15 protein.

The SIRPα gene promoter can be an endogenous non-human SIRPα gene promoter. For example, the SIRPα gene promoter can be the endogenous non-human SIRPα gene promoter at the non-human animal SIRPα gene locus. Where the SIRPα gene promoter is the endogenous non-human SIRPα gene promoter at the non-human animal SIRPα gene locus, the genetically modified non-human animal can include a null mutation in the non-human SIRPα gene at the non-human animal SIRPα gene locus. In one such embodiment, the genetically modified non-human animal is a mouse and the null mutation is a deletion of at least mouse SIRPα exons 2-4. In another such embodiment, the genetically modified non-human animal is heterozygous for the allele including the nucleic acid sequence that encodes the human SIRPα protein. In another such embodiment, the genetically modified non-human animal is homozygous for the allele including the nucleic acid sequence that encodes the human SIRPα protein.

In another embodiment of the first aspect, or in a further embodiment of any of the above embodiments thereof, the nucleic acid sequence that encodes the human SIRPα protein includes human SIRPα genomic coding and non-coding sequence.

In another embodiment of the first aspect, or in a further embodiment of any of the above embodiments thereof, the human SIRPα protein is a functional fragment of a full length human SIRPα protein. In one such embodiment, the functional fragment includes an extracellular domain of human SIRPα, e.g., an extracellular domain that includes at least amino acids 28-362 of SEQ ID NO:12.

In another embodiment of the first aspect, or in a further embodiment of any of the above embodiments thereof, the IL-15 gene promoter is an endogenous non-human IL-15 gene promoter. In one such embodiment, the IL-15 gene promoter is the endogenous non-human IL-15 gene promoter at the non-human animal IL-15 gene locus. In one embodiment, where the IL-15 gene promoter is the endogenous non-human IL-15 gene promoter at the non-human animal IL-15 gene locus, the genetically modified non-human animal includes a null mutation in the non-human IL-15 gene at the non-human animal IL-15 gene locus. In one such embodiment, the genetically modified non-human animal is a mouse and the null mutation is a deletion of at least mouse IL-15 exons 5-8. In another such embodiment, the genetically modified non-human animal is heterozygous for the allele including the nucleic acid sequence that encodes the human IL-15 protein. In another such embodiment, the genetically modified non-human animal is homozygous for the allele including the nucleic acid sequence that encodes the human IL-15 protein.

In another embodiment of the first aspect, or in a further embodiment of any of the above embodiments thereof, the nucleic acid sequence that encodes the human IL-15 protein includes human IL-15 genomic coding and non-coding sequence.

In another embodiment of the first aspect, or in a further embodiment of any of the above embodiments thereof, the human IL-15 protein is a functional fragment of a full length human IL-15 protein.

In another embodiment of the first aspect, or in a further embodiment of any of the above embodiments thereof, the genetically modified non-human animal is immunodeficient. For example, in one embodiment the genetically modified non-human animal includes a Rag2 gene knock-out. In another embodiment, the genetically modified non-human animal includes an an IL2rg gene knock-out or both a Rag2 gene knock-out and an an IL2rg gene knock-out.

In another embodiment of the first aspect, or in a further embodiment of any of the above embodiments thereof, the non-human animal is a mammal. In one such embodiment, the mammal is a rodent, e.g., a mouse.

In another embodiment of the first aspect, or in a further embodiment of any of the above embodiments thereof, the genetically modified non-human animal includes an engraftment of human hematopoietic cells. In one such embodiment, the genetically modified non-human animal includes an infection with a human pathogen. In one embodiment, where the the genetically modified non-human animal includes an infection with a human pathogen, the human pathogen activates, induces and/or targets T cells and/or natural killer (NK) cells. In another embodiment, where the the genetically modified non-human animal includes an infection with a human pathogen, the human pathogen is a pathogen that affects (e.g., by infecting) human intestine. In one such embodiment, the human pathogen is a human rotavirus. In another embodiment, where the the genetically modified non-human animal includes an infection with a human pathogen, the pathogen affects (e.g., by infecting) human lung. In one such embodiment, the human pathogen is an influenza virus. In another embodiment, where the the genetically modified non-human animal includes an infection with a human pathogen, the pathogen affects (e.g., by infecting) human liver. In yet another embodiment, a genetically modified non-human animal includes an engraftment of human hematopoietic cells and a tumor, e.g., a human tumor, e.g., transplanted human tumor.

In a second aspect, the present disclosure provides an in vivo model, including a genetically modified non-human animal including: a nucleic acid sequence incorporated into the genome of the genetically modified non-human animal, which sequence encodes a human SIRPα protein and is operably linked to a SIRPα gene promoter; a nucleic acid sequence incorporated into the genome of the genetically modified non-human animal, which sequence encodes a human IL-15 protein and is operably linked to an IL-15 gene promoter; and an engraftment of human hematopoietic cells, wherein the genetically modified non-human animal (i) expresses the human SIRPα protein and the human IL-15 protein, and (ii) includes human intraepithelial lymphocytes (IELs) in the small intestine and Peyer's patches of the genetically modified non-human animal.

In one embodiment of the second aspect, the genetically modified non-human animal includes an infection with a human pathogen, e.g., an intestinal pathogen. In one such embodiment, the intestinal pathogen is selected from: Campylobacter jejuni, Clostridium difficile, Enterococcus faecalis, Enterococcus faecium, Escherichia coli, Human Rotavirus, Listeria monocytogenes, Norwalk Virus, Salmonella enterica, Shigella flexneri, Shigella sonnei, Shigella dysenteriae, Yersinia pestis, Yersinia enterocolitica, and Helicobacter pylori.

In another embodiment of the second aspect, or in a further embodiment of any of the above embodiments thereof, the SIRPα gene promoter is an endogenous non-human SIRPα gene promoter. In one such embodiment, the SIRPα gene promoter is the endogenous non-human SIRPα gene promoter at the non-human animal SIRPα gene locus. In one embodiment, where the SIRPα gene promoter is the endogenous non-human SIRPα gene promoter at the non-human animal SIRPα gene locus, the genetically modified non-human animal includes a null mutation in the non-human SIRPα gene at the non-human animal SIRPα gene locus. In one such embodiment, the genetically modified non-human animal is a mouse and the null mutation is a deletion of at least mouse SIRPα exons 2-4. In another such embodiment, the genetically modified non-human animal is heterozygous for the allele including the nucleic acid sequence that encodes the human SIRPα protein. In another such embodiment, the genetically modified non-human animal is homozygous for the allele including the nucleic acid sequence that encodes the human SIRPα protein.

In another embodiment of the second aspect, or in a further embodiment of any of the above embodiments thereof, the nucleic acid sequence that encodes the human SIRPα protein includes human SIRPα genomic coding and non-coding sequence.

In another embodiment of the second aspect, or in a further embodiment of any of the above embodiments thereof, the human SIRPα protein is a functional fragment of a full length human SIRPα protein. In one such embodiment, the functional fragment includes an extracellular domain of human SIRPα, e.g., an extracellular domain that includes amino acids 28-362 of SEQ ID NO: 12.

In another embodiment of the second aspect, or in a further embodiment of any of the above embodiments thereof, the IL-15 gene promoter is an endogenous non-human IL-15 gene promoter. In one such embodiment, the IL-15 gene promoter is the endogenous non-human IL-15 gene promoter at the non-human animal IL-15 gene locus. In one embodiment, where the IL-15 gene promoter is the endogenous non-human IL-15 gene promoter at the non-human animal IL-15 gene locus, the genetically modified non-human animal includes a null mutation in the non-human IL-15 gene at the non-human animal IL-15 gene locus. In one such embodiment, the genetically modified non-human animal is a mouse and the null mutation is a deletion of at least mouse IL-15 exons 5-8. In one such embodiment, the genetically modified non-human animal is heterozygous for the allele including the nucleic acid sequence that encodes the human IL-15 protein. In another such embodiment, the genetically modified non-human animal is homozygous for the allele including the nucleic acid sequence that encodes the human IL-15 protein.

In another embodiment of the second aspect, or in a further embodiment of any of the above embodiments thereof, the nucleic acid sequence that encodes the human IL-15 protein includes human IL-15 genomic coding and non-coding sequence.

In another embodiment of the second aspect, or in a further embodiment of any of the above embodiments thereof, the human IL-15 protein is a functional fragment of a full length human IL-15 protein.

In another embodiment of the second aspect, or in a further embodiment of any of the above embodiments thereof, the genetically modified non-human animal is immunodeficient. For example, in one embodiment the genetically modified non-human animal includes a Rag2 gene knock-out. In another embodiment, the genetically modified non-human animal includes an an IL2rg gene knock-out or both a Rag2 gene knock-out and an an IL2rg gene knock-out.

In another embodiment of the second aspect, or in a further embodiment of any of the above embodiments thereof, the non-human animal is a mammal. In one such embodiment, the mammal is a rodent, e.g., a mouse.

In a third aspect, the present disclosure provides an in vivo model, including a genetically modified non-human animal including: a nucleic acid sequence incorporated into the genome of the genetically modified non-human animal, which sequence encodes a human SIRPα protein and is operably linked to a SIRPα gene promoter; a nucleic acid sequence incorporated into the genome of the genetically modified non-human animal, which sequence encodes a human IL-15 protein and is operably linked to an IL-15 gene promoter; and an engraftment of human hematopoietic cells, wherein the genetically modified non-human animal (i) expresses the human SIRPα protein and the human IL-15 protein, and (ii) includes human intraepithelial lymphocytes (IELs) in the lung of the genetically modified non-human animal.

In one embodiment of the third aspect, the genetically modified non-human animal includes an infection with a human pathogen, e.g., a lung pathogen. In one such embodiment, the lung pathogen is selected from: Streptococcus pyogenes, Haemophilus influenza, Corynebacterium diphtheria, SARS coronavirus, Bordetella pertussis, Moraxella catarrhalis, Influenza virus (A, B, C), Coronavirus, Adenovirus, Respiratory Syncytial Virus, Parainfluenza virus, Mumps virus, Streptococcus pneumoniae, Staphylococcus aureus, Legionella pneumophila, Klebsiella pneumoniae, Pseudomonas aeruginosa, Mycoplasma pneumonia, Mycobacterium tuberculosis, Chlamydia Pneumoniae, Blastomyces dermatitidis, Cryptococcus neoformans, and Aspergillus fumigatus.

In another embodiment of the third aspect, or in a further embodiment of any of the above embodiments thereof, the SIRPα gene promoter is an endogenous non-human SIRPα gene promoter. In one such embodiment, the SIRPα gene promoter is the endogenous non-human SIRPα gene promoter at the non-human animal SIRPα gene locus. In one embodiment, where the SIRPα gene promoter is the endogenous non-human SIRPα gene promoter at the non-human animal SIRPα gene locus, the genetically modified non-human animal includes a null mutation in the non-human SIRPα gene at the non-human animal SIRPα gene locus. In one such embodiment, the genetically modified non-human animal is a mouse and the null mutation is a deletion of at least mouse SIRPα exons 2-4. In another such embodiment, the genetically modified non-human animal is heterozygous for the allele including the nucleic acid sequence that encodes the human SIRPα protein. In another such embodiment, the genetically modified non-human animal is homozygous for the allele including the nucleic acid sequence that encodes the human SIRPα protein.

In another embodiment of the third aspect, or in a further embodiment of any of the above embodiments thereof, the nucleic acid sequence that encodes the human SIRPα protein includes human SIRPα genomic coding and non-coding sequence.

In another embodiment of the third aspect, or in a further embodiment of any of the above embodiments thereof, the human SIRPα protein is a functional fragment of a full length human SIRPα protein. In one such embodiment, the functional fragment includes an extracellular domain of human SIRPα, e.g., an extracellular domain including at least amino acids 28-362 of SEQ ID NO:12.

In another embodiment of the third aspect, or in a further embodiment of any of the above embodiments thereof, the IL-15 gene promoter is an endogenous non-human IL-15 gene promoter. In one such embodiment, the IL-15 gene promoter is the endogenous non-human IL-15 gene promoter at the non-human animal IL-15 gene locus.

In one embodiment, where the IL-15 gene promoter is the endogenous non-human IL-15 gene promoter at the non-human animal IL-15 gene locus, the genetically modified non-human animal includes a null mutation in the non-human IL-15 gene at the non-human animal IL-15 gene locus. In one such embodiment, the genetically modified non-human animal is a mouse and the null mutation is a deletion of at least mouse IL-15 exons 5-8. In another such embodiment, the genetically modified non-human animal is heterozygous for the allele including the nucleic acid sequence that encodes the human IL-15 protein. In another such embodiment, the genetically modified non-human animal is homozygous for the allele including the nucleic acid sequence that encodes the human IL-15 protein.

In another embodiment of the third aspect, or in a further embodiment of any of the above embodiments thereof, the nucleic acid sequence that encodes the human IL-15 protein includes human IL-15 genomic coding and non-coding sequence.

In another embodiment of the third aspect, or in a further embodiment of any of the above embodiments thereof, the human IL-15 protein is a functional fragment of a full length human IL-15 protein.

In another embodiment of the third aspect, or in a further embodiment of any of the above embodiments thereof, the genetically modified non-human animal is immunodeficient. For example, in one embodiment the genetically modified non-human animal includes a Rag2 gene knock-out. In another embodiment, the genetically modified non-human animal includes an an IL2rg gene knock-out or both a Rag2 gene knock-out and an an IL2rg gene knock-out.

In another embodiment of the third aspect, or in a further embodiment of any of the above embodiments thereof, the non-human animal is a mammal. In one such embodiment, the mammal is a rodent, e.g., a mouse.

In a fourth aspect, the present disclosure provides a method of determining the efficacy of a candidate T-cell inducing vaccine, the method including: administering a candidate T-cell inducing vaccine to a genetically modified non-human animal, wherein the genetically modified non-human animal is deficient for an endogenous immune system and includes: (i) a nucleic acid sequence incorporated into the genome of the genetically modified non-human animal, which sequence encodes a human SIRPα protein and is operably linked to a SIRPα gene promoter, (ii) a nucleic acid sequence incorporated into the genome of the genetically modified non-human animal, which sequence encodes a human IL-15 protein and is operably linked to an IL-15 gene promoter, and (iii) an engraftment of human hematopoietic cells, wherein the genetically modified non-human animal expresses the human SIRPα protein and the human IL-15 protein; challenging the genetically modified non-human animal with a human pathogen; and determining whether the candidate T-cell inducing vaccine induces a T cell mediated immune response in the genetically modified non-human animal.

In one embodiment of the fourth aspect, the SIRPα gene promoter is an endogenous non-human SIRPα gene promoter. In one such embodiment, the SIRPα gene promoter is the endogenous non-human SIRPα gene promoter at the non-human animal SIRPα gene locus. In one embodiment, where the SIRPα gene promoter is the endogenous non-human SIRPα gene promoter at the non-human animal SIRPα gene locus, the genetically modified non-human animal includes a null mutation in the non-human SIRPα gene at the non-human animal SIRPα gene locus. In one such embodiment, the genetically modified non-human animal is a mouse and the null mutation is a deletion of at least mouse SIRPα exons 2-4. In another such embodiment, the genetically modified non-human animal is heterozygous for the allele including the nucleic acid sequence that encodes the human SIRPα protein. In another such embodiment, the genetically modified non-human animal is homozygous for the allele including the nucleic acid sequence that encodes the human SIRPα protein.

In another embodiment of the fourth aspect, or in a further embodiment of any of the above embodiments thereof, the nucleic acid sequence that encodes the human SIRPα protein includes human SIRPα genomic coding and non-coding sequence.

In another embodiment of the fourth aspect, or in a further embodiment of any of the above embodiments thereof, the human SIRPα protein is a functional fragment of a full length human SIRPα protein. In one such embodiment, the functional fragment includes an extracellular domain of human SIRPα, e.g., an extracellular domain including at least amino acids 28-362 of SEQ ID NO:12.

In another embodiment of the fourth aspect, or in a further embodiment of any of the above embodiments thereof, the IL-15 gene promoter is an endogenous non-human IL-15 gene promoter. In one such embodiment, the IL-15 gene promoter is the endogenous non-human IL-15 gene promoter at the non-human animal IL-15 gene locus. In one embodiment, where the IL-15 gene promoter is the endogenous non-human IL-15 gene promoter at the non-human animal IL-15 gene locus, the genetically modified non-human animal includes a null mutation in the non-human IL-15 gene at the non-human animal IL-15 gene locus. In one such embodiment, the genetically modified non-human animal is a mouse and the null mutation is a deletion of at least mouse IL-15 exons 5-8. In another such embodiment, the genetically modified non-human animal is heterozygous for the allele including the nucleic acid sequence that encodes the human IL-15 protein. In another such embodiment, the genetically modified non-human animal is homozygous for the allele including the nucleic acid sequence that encodes the human IL-15 protein.

In another embodiment of the fourth aspect, or in a further embodiment of any of the above embodiments thereof, the nucleic acid sequence that encodes the human IL-15 protein includes human IL-15 genomic coding and non-coding sequence.

In another embodiment of the fourth aspect, or in a further embodiment of any of the above embodiments thereof, the human IL-15 protein is a functional fragment of a full length human IL-15 protein.

In another embodiment of the fourth aspect, or in a further embodiment of any of the above embodiments thereof, the genetically modified non-human animal includes a Rag2 gene knock-out.

In another embodiment of the fourth aspect, or in a further embodiment of any of the above embodiments thereof, the genetically modified non-human animal includes an IL2rg gene knock-out.

In another embodiment of the fourth aspect, or in a further embodiment of any of the above embodiments thereof, the genetically modified non-human animal is a mammal, such as a rodent, e.g., a mouse.

In a fifth aspect, the present disclosure provides a method of identifying an agent that inhibits an infection by a pathogen that activates, induces and/or targets human T cells and/or natural killer (NK) cells, the method including: administering an agent to an genetically modified non-human animal, wherein the genetically modified non-human animal is deficient for an endogenous immune system and includes: (i) a nucleic acid sequence incorporated into the genome of the genetically modified non-human animal, which sequence encodes a human SIRPα protein and is operably linked to a SIRPα gene promoter, (ii) a nucleic acid sequence incorporated into the genome of the genetically modified non-human animal, which sequence encodes a human IL-15 protein and is operably linked to an IL-15 gene promoter, (iii) an engraftment of human hematopoietic cells, and (iv) an infection by a pathogen that activates, induces and/or targets human T cells and/or natural killer cells, wherein the genetically modified non-human animal expresses the human SIRPα protein and the human IL-15 protein; and determining whether the agent reduces the amount of the pathogen in the pathogen-infected non-human animal.

In one embodiment of the fifth aspect, the SIRPα gene promoter is an endogenous non-human SIRPα gene promoter. In one such embodiment, the SIRPα gene promoter is the endogenous non-human SIRPα gene promoter at the non-human animal SIRPα gene locus. In one embodiment, where the SIRPα gene promoter is the endogenous non-human SIRPα gene promoter at the non-human animal SIRPα gene locus, the genetically modified non-human animal includes a null mutation in the non-human SIRPα gene at the non-human animal SIRPα gene locus. In one such embodiment, the genetically modified non-human animal is a mouse and the null mutation is a deletion of at least mouse SIRPα exons 2-4. In another such embodiment, the genetically modified non-human animal is heterozygous for the allele including the nucleic acid sequence that encodes the human SIRPα protein. In another such embodiment, the genetically modified non-human animal is homozygous for the allele including the nucleic acid sequence that encodes the human SIRPα protein.

In another embodiment of the fifth aspect, or in a further embodiment of any of the above embodiments thereof, the nucleic acid sequence that encodes the human SIRPα protein includes human SIRPα genomic coding and non-coding sequence.

In another embodiment of the fifth aspect, or in a further embodiment of any of the above embodiments thereof, the human SIRPα protein is a functional fragment of a full length human SIRPα protein. In one such embodiment, the functional fragment includes an extracellular domain of human SIRPα, e.g., an extracellular domain which includes amino acids 28-362 of SEQ ID NO:12.

In another embodiment of the fifth aspect, or in a further embodiment of any of the above embodiments thereof, the IL-15 gene promoter is an endogenous non-human IL-15 gene promoter. In one such embodiment, the IL-15 gene promoter is the endogenous non-human IL-15 gene promoter at the non-human animal IL-15 gene locus. In one embodiment, where the IL-15 gene promoter is the endogenous non-human IL-15 gene promoter at the non-human animal IL-15 gene locus, the genetically modified non-human animal includes a null mutation in the non-human IL-15 gene at the non-human animal IL-15 gene locus. In one such embodiment, the genetically modified non-human animal is a mouse and the null mutation is a deletion of at least mouse IL-15 exons 5-8. In another such embodiment, the genetically modified non-human animal is heterozygous for the allele including the nucleic acid sequence that encodes the human IL-15 protein. In another such embodiment, the genetically modified non-human animal is homozygous for the allele including the nucleic acid sequence that encodes the human IL-15 protein.

In another embodiment of the fifth aspect, or in a further embodiment of any of the above embodiments thereof, the nucleic acid sequence that encodes the human IL-15 protein includes human IL-15 genomic coding and non-coding sequence.

In another embodiment of the fifth aspect, or in a further embodiment of any of the above embodiments thereof, the human IL-15 protein is a functional fragment of a full length human IL-15 protein.

In another embodiment of the fifth aspect, or in a further embodiment of any of the above embodiments thereof, the genetically modified non-human animal includes a Rag2 gene knock-out.

In another embodiment of the fifth aspect, or in a further embodiment of any of the above embodiments thereof, the genetically modified non-human animal includes an IL2rg gene knock-out.

In another embodiment of the fifth aspect, or in a further embodiment of any of the above embodiments thereof, the genetically modified non-human animal is a mammal, such as a rodent, e.g., a mouse.

In a sixth aspect, the present disclosure provides a method of making a non-human animal expressing a human IL-15 protein and a human SIRPα protein, including: introducing into a genome of a first non-human animal a nucleic acid sequence encoding a human IL-15 protein, wherein the sequence encoding the human IL-15 protein is operably linked to an IL-15 gene promoter sequence; introducing into a genome of a second non-human animal a nucleic acid sequence encoding a human SIPRα protein, wherein the sequence encoding the human SIRPα protein is operably linked to a SIRPα promoter sequence; and making a third non-human animal that includes the nucleic acid sequence encoding the human IL-15 protein and the nucleic acid sequence encoding the human SIRPα protein, wherein the third non-human animal expresses the human IL-15 protein and the human SIPRα protein.

In one embodiment of the sixth aspect, the steps of introducing include generating a non-human animal from a pluripotent stem cell including the nucleic acid encoding human IL-15 or human SIRPα.

In another embodiment of the sixth aspect, or in a further embodiment of any of the above embodiments thereof, the first animal is a different animal than the second animal, and the step of making the third animal includes breeding the first and the second animal.

In another embodiment of the sixth aspect, the first animal and the second animal are the same, the step of introducing into the genome of the first animal includes contacting a first pluripotent stem cell with the nucleic acid sequence encoding the human IL-15 protein to obtain a second pluripotent stem cell, the step of introducing into the genome of the second animal includes contacting the second pluripotent stem cell with the nucleic acid sequence encoding the human SIRPα protein to obtain a third pluripotent step cell, and the third non-human animal is made from the third pluripotent stem cell.

In an alternative version of the sixth aspect, the present disclosure provides a method of making a non-human animal expressing a human IL-15 protein and a human SIRPα protein, including: introducing into a genome of a first non-human animal a nucleic acid sequence encoding a human SIPRα protein, wherein the sequence encoding the human SIPRα protein is operably linked to an SIPRα gene promoter sequence; introducing into a genome of a second non-human animal a nucleic acid sequence encoding a human IL-15 protein, wherein the sequence encoding the human IL-15 protein is operably linked to a IL-15 promoter sequence; and making a third non-human animal that includes the nucleic acid sequence encoding the human IL-15 protein and the nucleic acid sequence encoding the human SIRPα protein, wherein the third non-human animal expresses the human IL-15 protein and the human SIPRα protein.

In yet another embodiment of the sixth aspect, the first animal and the second animal are the same, the step of introducing into the genome of the first animal includes contacting a first pluripotent stem cell with the nucleic acid sequence encoding the human SIRPα protein to obtain a second pluripotent stem cell, the step of introducing into the genome of the second animal includes contacting the second pluripotent stem cell with the nucleic acid sequence encoding the human IL-15 protein to obtain a third pluripotent step cell, and the third non-human animal is made from the third pluripotent stem cell.

In another embodiment of the sixth aspect, or in a further embodiment of any of the above embodiments thereof, the pluripotent stem cell is an ES cell or an iPS cell.

In another embodiment of the sixth aspect, or in a further embodiment of any of the above embodiments thereof, the pluripotent stem cell is deficient for Rag2.

In another embodiment of the sixth aspect, or in a further embodiment of any of the above embodiments thereof, the pluripotent stem cell is deficient for IL2rg.

In another embodiment of the sixth aspect, or in a further embodiment of any of the above embodiments thereof, the third non-human animal is deficient in one or both of Rag2 and IL2rg.

In another embodiment of the sixth aspect, or in a further embodiment of any of the above embodiments thereof, the IL-15 promoter sequence is a sequence for the human IL-15 promoter.

In another embodiment of the sixth aspect, or in a further embodiment of any of the above embodiments thereof, the IL-15 promoter sequence is a sequence for the endogenous non-human animal IL-15 promoter.

In another embodiment of the sixth aspect, or in a further embodiment of any of the above embodiments thereof, the integration results in a replacement of the non-human IL-15 gene at the non-human IL-15 gene locus.

In another embodiment of the sixth aspect, or in a further embodiment of any of the above embodiments thereof, the nucleic acid sequence that encodes the human IL-15 protein includes human IL-15 genomic coding and non-coding sequence.

In a seventh aspect, the present disclosure provides a method of engrafting a genetically modified non-human animal expressing a human IL-15 protein, including: transplanting a population of cells including human hematopoietic cells into the genetically modified non-human animal made by a method according to the sixth aspect or any embodiment thereof. In one such embodiment, the transplanting includes tail-vein injection, fetal liver injection, or retro-orbital injection.

In another embodiment of the seventh aspect, or in a further embodiment of any of the above embodiments thereof, the genetically modified non-human animal is sublethally irradiated prior to transplantation.

In another embodiment of the seventh aspect, or in a further embodiment of any of the above embodiments thereof, the human hematopoietic cells are CD34+ cells.

In another embodiment of the seventh aspect, or in a further embodiment of any of the above embodiments thereof, the human hematopoietic cells are from fetal liver, adult bone marrow, or umbilical cord blood.

In an eighth aspect, the present disclosure provides a method of determining the efficacy of a candidate therapeutic antibody or antigen-binding protein in killing a target cell, the method including: administering the candidate therapeutic antibody or antigen-binding protein to a genetically modified non-human animal, wherein the genetically modified non-human animal is deficient for an endogenous immune system and includes: (i) a nucleic acid sequence incorporated into the genome of the genetically modified non-human animal, which sequence encodes a human SIRPα protein and is operably linked to a SIRPα gene promoter, (ii) a nucleic acid sequence incorporated into the genome of the genetically modified non-human animal, which sequence encodes a human IL-15 protein and is operably linked to an IL-15 gene promoter, and (iii) an engraftment of human hematopoietic cells, wherein the genetically modified non-human animal expresses the human SIRPα protein and the human IL-15 protein; and determining whether the candidate therapeutic antibody or antigen-binding protein modulates an NK cell mediated antibody-dependent cellular cytotoxicity against the target cell in the genetically modified non-human animal.

In a ninth aspect, the present disclosure provides a method of determining the efficacy of a candidate therapeutic antibody or antigen-binding protein, in killing a target cell including: isolating an NK cell from a genetically modified non-human animal, wherein the genetically modified non-human animal is deficient for an endogenous immune system and includes: (i) a nucleic acid sequence incorporated into the genome of the genetically modified non-human animal, which sequence encodes a human SIRPα protein and is operably linked to a SIRPα gene promoter, (ii) a nucleic acid sequence incorporated into the genome of the genetically modified non-human animal, which sequence encodes a human IL-15 protein and is operably linked to an IL-15 gene promoter, and (iii) an engraftment of human hematopoietic cells, wherein the genetically modified non-human animal expresses the human SIRPα protein and the human IL-15 protein; contacting the isolated NK cell with the candidate therapeutic antibody or antigen-binding protein and the target cell; and determining the antibody- or the antigen-binding protein-dependent cytolytic activity of the isolated NK cell against the target cell.

In a tenth aspect, the present disclosure provides a method of screening a candidate therapeutic antibody or antigen-binding protein for improved efficacy in killing a target cell including: administering the candidate therapeutic antibody or antigen-binding protein to a genetically modified non-human animal, wherein the genetically modified non-human animal is deficient for an endogenous immune system and includes: (i) a nucleic acid sequence incorporated into the genome of the genetically modified non-human animal, which sequence encodes a human SIRPα protein and is operably linked to a SIRPα gene promoter, (ii) a nucleic acid sequence incorporated into the genome of the genetically modified non-human animal, which sequence encodes a human IL-15 protein and is operably linked to an IL-15 gene promoter, and (iii) an engraftment of human hematopoietic cells, wherein the genetically modified non-human animal expresses the human SIRPα protein and the human IL-15 protein; and determining whether the candidate therapeutic antibody or antigen-binding protein displays improved efficacy in killing the target cell in the genetically modified non-human animal.

In an embodiment of any one of the eighth, ninth and tenth aspects, the target cell is one or more of a tumor cell, a virally-infected cell, a bacterially-infected cell, a bacterial cell, a fungal cell, and a parasitic cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides a schematic representation of replacement of the mouse SIRPα gene with human SIRPα sequence. FIG. 1 (top) shows the mouse Sirpa locus indicating the relative location of exons 1-8. FIG. 1 (bottom) provides a schematic representation showing the final targeted allele with human exons 2-4. The encoded chimeric protein possesses an extracellular region corresponding to amino acids 28-362 of the wild-type human SIRPα protein fused to the intracellular portion of the mouse SIRPα protein. Diagonally striped shapes represent inserted human sequence.

FIG. 2 provides a schematic representation illustrating targeted genomic replacement of the mouse IL-15 gene as achieved for mouse 2. Empty shapes represent inserted human sequence.

FIG. 3A provides graphs showing hIL-15 gene expression in various tissues of non-engrafted SRG (human SIRPα, Rag K O, IL-2rg KO) and SRG-15 (human SIRPα, Rag KO, IL-2rg KO, human IL-15 (mouse 1) mice. Y-axis shows level of hIL-15 mRNA relative to the housekeeping gene Hprt.

FIG. 3B provides graphs showing human hIL-15 gene expression in various tissues of non-engrafted RG (Rag KO, IL-2rg KO) and non-engrafted SRG-15 (human SIRPα, Rag KO, IL-2rg KO, human IL-15) mice (mouse #1 and mouse #2 as indicated).

FIG. 4 provides serum levels of human IL-15 protein in SRG, SRG IL-15 h/m (mouse 2) and SRG IL-15 h/h (mouse 2) mice after challenge with poly (I:C).

FIG. 5A provides a graph showing efficient engraftment of human hematopoietic cells in the blood of NSG, SRG and SRG-15 (mouse 2) mice 12-14 weeks post engraftment. All data are shown as mean±s.e.m. Statistical analyses were performed using unpaired, two-tailed Mann-Whitney U-test (* P<0.05, ** P<0.01, **** P<0.0001).

FIG. 5B provides graphs showing human CD45+ cell numbers in the BM, spleen, LN, liver and lung of SRG and SRG-15 (mouse 2) 14 weeks post engraftment.

FIG. 6A provides plots showing human T and NK cell frequencies in SRG and SRG-15 mice (mouse 1) in bone marrow (BM), liver, and lung.

FIG. 6B provides graphs showing human NK cell frequencies in SRG and SRG-15 mice (mouse 1) in various tissues.

FIG. 6C provides plots and graphs illustrating human NK cell maturation in the liver of SRG and SRG-15 mice (mouse 1).

FIG. 6D provides plots showing that human CD56^{dim} CD16⁺ NK cells express high levels of human killer inhibitory receptors in the spleen of SRG-15 mice.

FIG. 7A provides a graph showing the frequency of human NK cells in the blood of NSG, SRG and SRG-15 (mouse 2) mice 10-12 weeks post engraftment. All data are shown as mean±s.e.m. Statistical analyses were performed using unpaired, two-tailed Mann-Whitney U-test (* P<0.05, ** P<0.01, **** P<0.0001).

FIG. 7B provides a graph showing the percentage of human NKp46⁺ cells in the spleen 14 weeks post engraftment for SRG, SRG-15^{h/m}, and SRG-15^{h/h}. All data are shown as mean±s.e.m. Statistical analyses were performed using unpaired, two-tailed Mann-Whitney U-test (* P<0.05, ** P<0.01, **** P<0.0001).

FIG. 7C provides plots showing the frequency of human NK cells in the blood, spleen (SP), liver and lung of SRG and SRG-15 (mouse 2) mice 14 weeks post engraftment. All data are shown as mean±s.e.m. Statistical analyses were performed using unpaired, two-tailed Mann-Whitney U-test (* P<0.05, ** P<0.01, **** P<0.0001).

FIG. 7D provides graphs showing the frequency of human NK cells in the spleen (SP), liver and lung of SRG and SRG-15 (mouse 2) mice 14 weeks post engraftment. All data are shown as mean±s.e.m. Statistical analyses were performed using unpaired, two-tailed Mann-Whitney U-test (* P<0.05, ** P<0.01, **** P<0.0001).

FIG. 8 provides plots (left) showing human T and NK cell distribution in SRG and SRG-15 (mouse 2) mice in blood (gated on human CD45+ cells (hematopoietic cells) and NKp46+ cells (NK cells); and a graph (right) showing the percentage of the hCD45+ cells that are NKp46+ cells in the blood of engrafted SRG-15 mice.

FIG. 9A provides plots showing the distribution of NK cells and T cells in the spleen and graphs showing the percentage and number of NKp46+ cells in the spleen of SRG-15 mice (mouse 2) engrafted with CD34+ huHSCs relative to SRG mice engrafted with CD34+ huHSCs.

FIG. 9B provides a graph showing human immune cell composition in the blood of NSG (n=5), SRG (n=19) and SRG-15 (mouse 2) mice (n=39) 10-12 weeks post engraftment.

FIG. 9C provides human CD45+ cell numbers in the thymus of SRG and SRG-15 (mouse 2) mice 14 weeks post engraftment.

FIG. 9D provides representative flow cytometry plots of hCD45+ cells in the thymus of an SRG and SRG-15 (mouse 2) mouse.

FIG. 9E provides a graph showing the composition of hCD45+ cells in the thymus of SRG (n=8) and SRG-15 (mouse 2) mice (n=4) 14 weeks post engraftment.

FIG. 10A provides plots showing the requency of CD56^{bright} CD16⁻ and CD56^{dim} CD16⁺ NK cell subsets in the blood and spleen of SRG and SRG-15 (mouse 2) mice seven weeks post engraftment.

FIG. 10B provides graphs showing the requency of CD56^{bright} CD16⁻ and CD56^{dim} CD16⁺ NK cell subsets in the blood and spleen of SRG and SRG-15 (mouse 2) mice seven weeks post engraftment.

FIG. 10C provides plots and graphs showing expression of killer inhibitory receptors (KIRs) on NK cell subsets in humans and SRG-15 mice (mouse 2).

FIG. 11 provides two plots (top left and top right) showing the distribution of CD16⁺ vs. CD16 NK cells in the blood of SRG-15 mice (mouse 2) relative to a PBMC sample. FIG. 11 also provides a graph (bottom) showing the percentage of NKp46⁺ cells that are CD16⁺ vs. CD16 in either blood obtained from SRG-15 mice (mouse 2) or PBMC-derived sample.

FIG. 12 provides graphs showing human NK cell development in the bone marrow of SRG and SRG-15 (mouse 2) mice seven weeks post engraftment. All data are shown as mean±s.e.m. Statistical analyses were performed using unpaired, two-tailed Mann-Whitney U-test (* P<0.05, ** P<0.01, **** P<0.0001).

FIG. 13A provides graphs showing human T cell frequencies in SRG and SRG-15 mice (mouse 1) in various tissues. (K/μl=thousands of cells per μl).

FIG. 13B provides plots and graphs showing human CD8⁺ T cell phenotype in blood and liver for SRG and SRG-15 mice (mouse 1).

FIG. 14A provides plots and a graph showing expression of the tissue-resident marker CD69 in lung CD8⁺ T cells of SRG and SRG-15 (mouse 1) mice.

FIG. 14B provides a plot and a graph showing expression of the tissue-resident marker CD69 in liver CD8⁺ T cells of SRG and SRG-15 (mouse 1) mice.

FIG. 15A provides graphs showing the frequency of hCD3⁺ T cells in the spleen, lung and liver of SRG and SRG-15 (mouse 2) mice 16 weeks post engraftment.

FIG. 15B provides graphs showing the CD4/CD8 ratio in the spleen, lung and liver of SRG and SRG-15 (mouse 2) mice 16 weeks post engraftment.

FIG. 16A provides plots illustrating the frequency of human lamina propria lymphocytes (LPLs) in the colon of SRG and SRG-15 (mouse 1) mice.

FIG. 16B provides graphs illustrating the frequency of human lamina propria lymphocytes (LPLs) in the colon of SRG and SRG-15 (mouse 1) mice.

FIG. 17A together with FIGS. 17B-17C, illustrates efficient engraftment of human intraepithelial lymphocytes (IELs) in the small intestine of 16 week old SRG-15 mice (mouse 1). FIG. 17A provides plots and graphs showing human CD45⁺ cells and CD8+ T cells within the IEL fraction of SRG and SRG-15 (mouse 1) mice.

FIG. 17B provides images of immunohistochemical staining of hCD45 in the small intestine of 16 week old SRG and SRG-15 (mouse 1) mice.

FIG. 17C provides plots showing phenotypic characteristics of human CD8+ T cells in the spleen and small intestine of SRG-15 mice (mouse 1).

FIG. 18A provides representative FACS plots showing mouse and human CD45+ cells within the IEL fraction of SRG and SRG-15 (mouse 2) mice 16 weeks post engraftment.

FIG. 18B provides graphs showing the number of human IELs in the small intestine of SRG relative to SRG-15 (mouse 2) mice and the number of human LPLs in the large intestine SRG relative to SRG-15 (mouse 2) mice. All data are shown as mean±s.e.m. Statistical analyses were performed using unpaired, two-tailed Mann-Whitney U-test (*** P<0.001).

FIG. 18C provides a plot showing composition of hCD3+ cells in the small intestine of SRG-15 mice (mouse 2). One representative FACS plot of eight SRG-15 mice (mouse 2).

FIG. 18D provides graphs showing phenotypic characteristics of hCD3+ hCD8+ T cells in the spleen and small intestine of SRG-15 mice (mouse 2).

FIG. 18E provides images of immunohistochemical staining of hCD8 in the small intestine of SRG and SRG-15 (mouse 2) mice. The arrows indicate hCD8+ IELs. The pictures are representative of three mice per group.

FIG. 19A provides plots and graphs showing the distribution and the number of hCD45+ cells in the intraepithelial lymphocyte populations of SRG and SRG-15 mice and the relative percentages of NK cells and T cells in the populations of hCD45+ cells in the intraepithelial lymphocyte populations of SRG and SRG-15 (mouse 2) mice.

FIG. 19B provides plots and graphs showing the distribution and percentage of CD16+ and CD16− NK cells in intraepithelial lymphocytes of SRG-15 mice (mouse 2) as compared with blood and spleen.

FIG. 19C provides plots and graphs showing the distribution and numbers of human IELs and human lamina propria lymphocytes (LPLs) in SRG and SRG-15 (mouse 2) mice.

FIGS. 20A and 20B provides plots and graphs demonstrating the presence of discernible Peyer's Patches containing prodominantly hCD45+ cells in SRG-15 mice (mouse 2).

FIG. 21A provides a timeline for cohousing and feces sample collection for gut microbiota sequencing.

FIG. 21B provides a diagram showing the relative abundance of mouse bacteria in the gut of non-engrafted and engrafted SRG and SRG-15 (mouse 1) mice.

FIG. 22 illustrates the functional relevance of human tissue-resident T cells in SRG-15 mice. More specifically, FIG. 22 provides a graph demonstrating the functional relevance of human IELs in clearing acute rotavirus infection.

FIG. 23A provides ViSNE plots showing CyTOF-based analysis of 42 parameters of CD56^{bright} CD16⁻ and CD56^{dim} CD16⁺ NK cell subsets in humans (n=20) and SRG-15 mice (mouse 2) (n=9). Each dot represents a single cell.

FIG. 23B provides ViSNE plots showing the expression intensity of eight selected markers on CD56^{bright} CD16⁻ NK cells in humans (n=20) and SRG-15 mice (mouse 2) (n=9).

FIG. 23C ViSNE plots showing the expression intensity of eight selected markers on CD56^{dim} CD16⁺ NK cells in humans (n=20) and SRG-15 mice (n=9).

FIG. 24A provides a graph showing the percentage of blood NK cells in SRG vs SRG-15 (mouse 2) mice that are CD69+ before and after poly-IC injection.

FIG. 24B provides graphs showing IFNγ production from SRG and SRG-15 (mouse 2) derived NK cells after in vitro stimulation with poly I:C or human IL-12p70. NK cells from mice are compared against NK cells derived from healthy human PBMCs. All samples are normalized for NK number.

FIG. 24C provides graphs showing the cytolytic capacity of spenic NK cells from SRG and SRG-15 (mouse 2) mice either against HLA class I deficient K562 cells (left) or against Raji cells in the absence (top right) or the presence (bottom right) of anti-CD20 antibody. SRG-15 #1 and SRG-15 #2 represent two different NK cell preparations from SRG-15 (mouse 2) littermates.

FIG. 25A provides a graph showing that Human NK cells in SRG-15 mice (mouse 2) inhibit tumor growth following treatment with rituximab (RTX). All data are shown as mean±s.e.m. Statistical analyses were performed using unpaired, two-tailed Mann-Whitney U-test (*** P<0.001).

FIG. 25B provides plots and a graph showing the frequency of human NK cells and T cells in human tumor xenografts of untreated (n=5) and RTX-treated SRG-15 mice (n=1). All data are shown as mean±s.e.m. Statistical analyses were performed using unpaired, two-tailed Mann-Whitney U-test (*** P<0.001).

FIG. 25C provides plots and graphs showing human NK cell subsets in the blood and tumor of untreated (n=2) and RTX-treated SRG-15 mice (n=1). All data are shown as mean±s.e.m. Statistical analyses were performed using unpaired, two-tailed Mann-Whitney U-test (*** P<0.001).

### DETAILED DESCRIPTION

Before the present methods and compositions are described, it is to be understood that this invention is not limited to particular method or composition described, as such may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, particular methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth.

The publications discussed herein are provided solely for their disclosure prior to the filing date of the present application. Nothing herein is to be construed as an admission that the present invention is not entitled to antedate such publication.

Genetically modified non-human animals expressing human SIRPα and human IL-15 from the non-human animal genome are provided. Also provided are methods for making non-human animals expressing human SIRPα and human IL-15 from the non-human animal genome, and methods for using non-human animals expressing human SIRPα and human IL-15 from the non-human animal genome. These animals and methods find many uses in the art, including, for example, in modeling human T cell and/or natural killer (NK) cell development and function; in modeling human pathogen infection, e.g., human pathogen infection of specific tissues, e.g., human gut, lung or liver pathogen infection; in modeling human pathogen infection of human T cells and/or NK cells; in in vivo screens for agents that inhibit infection by a pathogen that activates, induces and/or targets T cells and/or NK cells; in in vivo screens for agents that modulate the development and/or function of human T cells and/or NK cells, e.g. in a healthy or a diseased state; in in vivo screens for agents that are toxic to human T cells and/or NK cells; in in vivo screens for agents that prevent against, mitigate, or reverse the toxic effects of toxic agents on human T cells and/or NK cells; in in vivo screens of candidate T cell-inducing vaccines; and in in vivo and in vitro screens for agents that inhibit tumor growth and/or infection by activating NK cell-mediated antibody dependent cellular cytotoxicity (ADCC) processes.

### Humanized SIRPα Non-Human Animals

In some aspects of the present disclosure, a humanized SIRPα non-human animal is provided. By a humanized SIRPα non-human animal, or “SIRPα non-human animal”, is meant a non-human animal including a nucleic acid sequence that encodes a human SIRPα protein. As used herein, “human SIRPα protein” means a protein that is a wild-type (or native) human SIRPα protein or a variant of a wild-type (or native) human SIRPα protein, which retains one or more signaling and/or receptor functions of a wild-type human SIRPα protein. As used herein, the term “variant” defines either an isolated naturally occurring genetic mutant of a human polypeptide or nucleic acid sequence or a recombinantly prepared variation of a human polypeptide or nucleic acid sequence, each of which contains one or more mutations compared with the corresponding wild-type human nucleic acid or polypeptide sequence. For example, such mutations can be one or more amino acid substitutions, additions, and/or deletions. The term “variant” also includes human homologs and orthologues. In some embodiments, a variant polypeptide of the present invention has 70% or more identity, e.g. 75%, 80%, or 85% or more identity to a wild-type human polypeptide, e.g. 90%, 91%, 92%, 93%, 94%, 95%, 96%, 97%, 98% or 99% identity to a wild-type human polypeptide.

The percent identity between two sequences may be determined using any convenient technique in the art, for example, aligning the sequences using, e.g., publicly available software. Mutations can be introduced using standard molecular biology techniques, such as site-directed mutagenesis, PCR-mediated mutagenesis, directed evolution, and the like. One of skill in the art will recognize that one or more nucleic acid substitutions can be introduced without altering the amino acid sequence, and that one or more amino acid mutations can be introduced without altering the functional properties of the human protein.

Conservative amino acid substitutions can be made in human proteins to produce human protein variants. By conservative amino acid substitutions it is meant art-recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

Human variants can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, methylhistidine, and ornithine.

Human variants will typically be encoded by nucleic acids having a high degree of identity with a nucleic acid encoding the wild-type human protein. The complement of a nucleic acid encoding a human variant specifically hybridizes with a nucleic acid encoding a wild-type human under high stringency conditions. Nucleic acids encoding a human variant can be isolated or generated recombinantly or synthetically using well-known methodology. Also encompassed by the term “human SIRPα protein” are fragments of a wild-type human SIRPα protein (or a variant thereof), which retain one or more signaling and/or receptor functions of a wild-type human SIRPα protein, e.g., an extracellular domain of a human SIRPα protein.

The term “human SIRPα protein” also encompasses fusion proteins, i.e., chimeric proteins, which include one or more fragments of a wild-type human SIRPα protein (or a variant thereof) and which retain one or more signaling and/or receptor functions of a wild-type human SIRPα protein. A fusion protein which includes one or more fragments of a wild-type human SIRPα protein (or a variant thereof), e.g., in combination with one or more non-human peptides or polypeptides, may also be referred to herein as a humanized SIRPα protein. Thus, for example, a protein which includes an amino acid sequence of an extracellular domain of a wild-type human SIRPα protein fused with a signaling domain of a wild-type mouse SIRPα protein is encompassed by the term “human SIRPα protein”.

In some instances, a human SIRPα protein accordingly to the present disclosure includes an amino acid sequence having at least about 70%, at least about 75%, at least about 80%, at least about 85%, at least about 90%, at least about 95%, at least about 98%, at least about 99%, or 100%, amino acid sequence identity to amino acids 28-362 of SEQ ID NO:12.

A nucleic acid sequence that encodes a human SIRPα protein is, therefore, a polynucleotide that includes a coding sequence for a human SIRPα protein, e.g., a wild-type human SIRPα protein, a variant of a wild-type human SIRPα protein, a fragment of a wild-type human SIRPα protein (or a variant thereof) which retains one or more signaling and/or receptor functions of a wild-type human SIRPα protein, or fusion proteins, i.e., chimeric proteins, which include one or more fragments of a wild-type human SIRPα protein (or a variant thereof) and which retain one or more signaling and/or receptor functions of a wild-type human SIRPα protein.

SIRPα (also known as “signal regulatory protein α” and “CD172A” in humans) is a member of the signal-regulatory-protein (SIRP) family, and also belongs to the immunoglobulin superfamily. SIRPα has been shown to improve cell engraftment in immunodeficient mice (Strowig et al. Proc Natl Acad Sci USA 2011; 108:13218-13223). Polypeptide sequence for wild-type human SIRPα and the nucleic acid sequence that encodes wild-type human SIRPα may be found at Genbank Accession Nos. NM_001040022.1 (variant 1), NM_001040023.1 (variant 2), and NM_080792.2 (variant 3). The SIRPα gene is conserved in at least chimpanzee, Rhesus monkey, dog, cow, mouse, rat, and chicken. The genomic locus encoding the wild-type human SIRPα protein may be found in the human genome at Chromosome 20; NC_000020.11 (1894117-1939896). Protein sequence is encoded by exons 1 through 8 at this locus. As such, in some embodiments, a nucleic acid sequence including coding sequence for human SIRPα includes one or more of exons 1-8 of the human SIRPα gene. In some instances, the nucleic acid sequence also includes aspects of the genomic locus of the human SIRPα, e.g., introns, 3′ and/or 5′ untranslated sequence (UTRs). In some instances, the nucleic acid sequence includes whole regions of the human SIRPα genomic locus. In some instances, the nucleic acid sequence includes exons 2-4 of the human SIRPα genomic locus.

In the humanized SIRPα non-human animals of the subject application, the nucleic acid sequence that encodes a human SIRPα protein is operably linked to one or more regulatory sequences of a SIRPα gene, e.g., a regulatory sequence of a SIRPα gene of the non-human animal. Non-human animal, e.g., mouse, SIRPα regulatory sequences are those sequences of the non-human animal SIRPα genomic locus that regulate the non-human animal SIRPα expression, for example, 5′ regulatory sequences, e.g., the SIRPα promoter, SIRPα 5′ untranslated region (UTR), etc.; 3′ regulatory sequences, e.g., the 3′UTR; and enhancers, etc.

A “promoter” or “promoter sequence” refers to a DNA regulatory region capable of binding RNA polymerase in a cell and initiating transcription of a downstream (3′ direction) coding sequence. The promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Of particular interest to the present disclosure are DNA regulatory elements, e.g. promoters, which promote the transcription of the human protein in the same spatial and temporal expression pattern, i.e., in the same cells and tissues and at the same times, as would be observed for the corresponding endogenous protein.

Mouse SIRPα is located on chromosome 2; NC_000068.7 (129592606-129632228), and the mouse SIRPα coding sequence may be found at Genbank Accession Nos. NM_007547.4 (isoform 1), NM_001177647.2 (isoform 2), NM_001291019.1 (isoform 3), NM_001291020.1 (isoform 3), NM_001291021.1 (isoform 4), NM_001291022.1 (isoform 5). The regulatory sequences of mouse SIRPα are well defined in the art, and may be readily identified using in silico methods, e.g., by referring to the above Genbank Accession Nos. on the UCSC Genome Browser on the world wide web, or by experimental methods as described in the art. In some instances, e.g., when the nucleic acid sequence that encodes a human SIRPα protein is located at the mouse SIRPα genomic locus, the regulatory sequences operably linked to the human SIRPα coding sequence are endogenous, or native, to the mouse genome, i.e., they were present in the mouse genome prior to integration of human nucleic acid sequences.

In some instances, the humanized SIRPα non-human animal, e.g., mouse, is generated by the random integration, or insertion, of a human nucleic acid sequence encoding a human SIRPα protein (including fragments as described above), i.e., a “human SIRPα nucleic acid sequence”, or “human SIRPα sequence”, into the genome. Typically, in such embodiments, the location of the nucleic acid sequence encoding a human SIRPα protein in the genome is unknown. In other instances, the humanized SIRPα non-human animal is generated by the targeted integration, or insertion, of human SIRPα nucleic acid sequence into the genome, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a humanized SIRPα mouse including a nucleic acid sequence that encodes a human SIRPα protein created by targeting human SIRPα nucleic acid sequence to the mouse SIRPα locus, human SIRPα nucleic acid sequence may replace some or all of the mouse sequence, e.g. exons and/or introns, at the SIRPα locus. In some such instances, a human SIRPα nucleic acid sequence is integrated into the mouse SIRPα locus such that expression of the human SIRPα sequence is regulated by the native, or endogenous, regulatory sequences at the mouse SIRPα locus. In other words, the regulatory sequence(s) to which the nucleic acid sequence encoding a human SIRPα protein is operably linked are the native SIRPα regulatory sequences at the mouse SIRPα locus.

In some instances, the integration of a human SIRPα sequence does not affect the transcription of the gene into which the human SIRPα sequence has integrated. For example, if the human SIRPα sequence integrates into a coding sequence as an intein, or the human SIRPα sequence includes a 2A peptide, the human SIRPα sequence will be transcribed and translated simultaneously with the gene into which the human SIRPα sequence has integrated. In other instances, the integration of the human SIRPα sequence interrupts the transcription of the gene into which the human SIRPα sequence has integrated. For example, upon integration of the human SIRPα sequence by homologous recombination, some or all of the coding sequence at the integration locus may be removed, such that the human SIRPα sequence is transcribed instead. In some such instances, the integration of a human SIRPα sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele is indistinguishable from a deletion of the entire locus.

In some instances, the humanized SIRPα non-human animal, e.g., mouse, includes one copy of the nucleic acid sequence encoding a human SIRPα protein. For example, the non-human animal may be heterozygous for the nucleic acid sequence. In other words, one allele at a locus will include the nucleic acid sequence, while the other will be the endogenous allele. For example, as discussed above, in some instances, a human SIRPα nucleic acid sequence is integrated into the non-human animal, e.g., mouse, SIRPα locus such that it creates a null allele for the non-human animal SIRPα. In some such embodiments, the humanized SIRPα non-human animal may be heterozygous for the nucleic acid sequence encoding human SIRPα, i.e., the humanized SIRPα non-human animal includes one null allele for the non-human animal SIRPα (the allele including the nucleic acid sequence) and one endogenous SIRPα allele (wild-type or otherwise). In other words, the non-human animal is a SIRPα h/m non-human animal, where “h” represents the allele including the human sequence and “m” represents the endogenous allele. In other instances, the humanized SIRPα includes two copies of the nucleic acid sequence encoding a human SIRPα protein. For example, the non-human animal, e.g., mouse, may be homozygous for the nucleic acid sequence, i.e., both alleles for a locus in the diploid genome will include the nucleic acid sequence, i.e., the humanized SIRPα non-human animal includes two null alleles for the non-human animal SIRPα (the allele including the nucleic acid sequence). In other words, the non-human animal is a SIRPα^{h/h} non-human animal.

In some embodiments, the humanized SIRPα non-human animal, e.g., mouse, includes other genetic modifications. In some embodiments, the humanized SIRPα non-human animal is an immunocompromised animal. For example, the humanized SIRPα non-human animal may include at least one null allele for the Rag2 gene (“recombination activating gene 2”, wherein the coding sequence for the mouse gene may be found at Genbank Accession No. NM_009020.3). In some embodiments, the humanized SIRPα non-human animal includes two null alleles for Rag2. In other words, the humanized SIRPα non-human animal is homozygous null for Rag2. As another example, the humanized SIRPα non-human animal includes at least one null allele for the IL2rg gene (“interleukin 2 receptor, gamma”, also known as the common gamma chain, or γC, wherein the coding sequence for the mouse gene may be found at Genbank Accession No. NM_013563.3). In some embodiments, the humanized SIRPα non-human animal includes two null alleles for IL2rg. In other words, the humanized SIRPα non-human animal is homozygous null for IL2rg, i.e., it is IL2rg^{-/-} (or IL2rg^{Y/-} where the IL2rg gene is located on the X chromosome as in mouse). In some embodiments, the SIRPα non-human animal includes a null allele for both Rag2 and IL2rg, i.e., it is Rag2^{-/-} IL2rg^{-/-} (or Rag2^{-/-} IL2rg^{Y/-} where the IL2rg gene is located on the X chromosome as in mouse). Other genetic modifications are also contemplated. For example, the humanized SIRPα non-human animal may include modifications in other genes associated with the development and/or function of hematopoietic cells and the immune system, e.g. the replacement of one or more other non-human animal genes with nucleic acid sequence encoding the human ortholog. Additionally or alternatively, the humanized SIRPα non-human animal may include modifications in genes associated with the development and/or function of other cells and tissues, e.g., genes associated with human disorders or disease, or genes that, when modified in a non-human animal, e.g., mice, provide for models of human disorders and disease.

### Humanized IL-15 Non-Human Animals

In some aspects of the present disclosure, a humanized IL-15 non-human animal is provided. By a humanized IL-15 non-human animal, or “IL-15 non-human animal”, is meant a non-human animal including a nucleic acid sequence that encodes a human IL-15 protein. As used herein, “human IL-15 protein”, means a protein that is a wild-type (or native) human IL-15 protein or a variant of a wild-type (or native) human IL-15 protein, which retains one or more signaling functions of a wild-type (or native) human IL-15 protein, e.g., which allows for stimulation of (or signaling via) the human IL-15 receptor, and/or which is capable of binding to the human IL-15 receptor alpha subunit of the human IL-15 receptor, and/or which is capable of binding to IL-2R beta/IL-15R beta and the common γ-chain (γc). Also encompassed by the term “human IL-15 protein” are fragments of a wild-type human IL-15 protein (or variants thereof), which retain one or more signaling functions of a wild-type human IL-15 protein, e.g., a fragment of a human IL-15 protein, which allows for stimulation of (or signaling via) the human IL-15 receptor, and/or which is capable of binding to the human IL-15 receptor alpha subunit of the human IL-15 receptor, and/or which is capable of binding to IL-2R beta/IL-15R beta and the common γ-chain (γc).

The term “human IL-15 protein” also encompasses fusion proteins, i.e., chimeric proteins, which include one or more fragments of a wild-type human IL-15 protein (or a variant thereof) and which retain one or more signaling functions of a wild-type human IL-15 protein, e.g., as described above. A fusion protein which includes one or more fragments of a wild-type human IL-15 protein (or a variant thereof) may also be referred to herein as a humanized IL-15 protein.

A nucleic acid sequence that encodes a human IL-15 protein is, therefore, a polynucleotide that includes a coding sequence for a human IL-15 protein, i.e., a wild-type human IL-15 protein, a variant of a wild-type human IL-15 protein, a fragment of a wild-type human IL-15 protein (or a variant thereof) which retains one or more signaling functions of a wild-type human IL-15 protein, or fusion proteins, i.e., chimeric proteins, which include one or more fragments of a wild-type human IL-15 protein (or a variant thereof) and which retain one or more signaling functions of a wild-type human IL-15 protein, e.g., as described above.

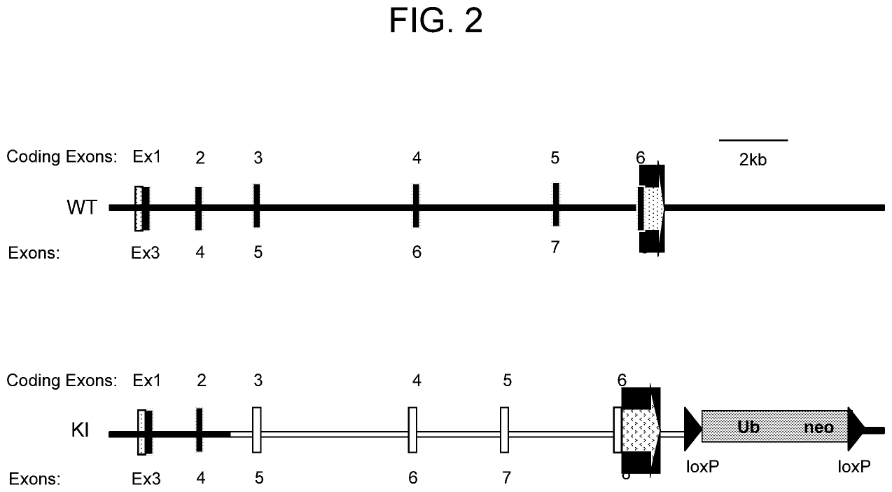

IL-15 (also known as “Interleukin 15”) is a cytokine that stimulates the proliferation of T lymphocytes. Polypeptide sequence for wild-type human IL-15 and the nucleic acid sequence that encodes wild-type human IL-15 may be found at Genbank Accession Nos. NM_000585.4; NP_000576.1 (isoform 1), NM_172175.2; NP_751915.1 (isoform 2). The genomic locus encoding the wild-type human IL-15 protein may be found in the human genome at Chromosome 4; NC_000004.12 (141636596-141733987). The human IL-15 locus includes 8 exons, with exons 3-8 being coding exons. As such, in some embodiments, a nucleic acid sequence including coding sequence for human IL-15 includes one or more of exons 3-8 of the human IL-15 gene (i.e., coding exons 1-6, see FIG. 2). For example, various IL-15 mRNA isoforms have been identified which are produced through the following exon usage combinations Exons 1-2-3-4-5-6-7-8; Exons 1-3-4-5-6-7-8 or Exons 1-3-4-(alternative exon 5)-5-6-7-8). In some instances, the nucleic acid sequence also includes aspects of the genomic locus of the human IL-15, e.g., introns, 3′ and/or 5′ untranslated sequence (UTRs). In some instances, the nucleic acid sequence includes whole regions of the human IL-15 genomic locus. In some instances, the nucleic acid sequence includes exons 5-8 of the human IL-15 genomic locus (i.e., coding exons 3-6).

In some instances, a human IL-15 protein accordingly to the present disclosure includes an amino acid sequence having at least about 70%, at least about 75%, at least about 80%, at least about 85%, at least about 90%, at least about 95%, at least about 98%, at least about 99%, or 100%, amino acid sequence identity to SEQ ID NO: 31.

In the humanized IL-15 non-human animals of the subject application, the nucleic acid sequence that encodes a human IL-15 protein is operably linked to one or more regulatory sequences of an IL-15 gene, e.g., a regulatory sequence of an IL-15 gene of the non-human animal. Non-human animal, e.g., mouse, IL-15 regulatory sequences are those sequences of the non-human animal IL-15 genomic locus that regulate the non-human animal IL-15 expression, for example, 5′ regulatory sequences, e.g., the IL-15 promoter, IL-15 5′ untranslated region (UTR), etc.; 3′ regulatory sequences, e.g., the 3′UTR; and enhancers, etc. Mouse IL-15 is located on Chromosome 8, NC_000074.6 (82331624-82403227, complement), and the mouse IL-15 coding sequence may be found at Genbank Accession Nos. NM_008357.2 (variant 1); NM_001254747.1 (variant 2). The regulatory sequences of mouse IL-15 are well defined in the art, and may be readily identified using in silico methods, e.g., by referring to the above Genbank Accession Nos. on the UCSC Genome Browser, on the world wide web at genome.ucsc.edu, or by experimental methods as described in the art. In some instances, e.g., when the nucleic acid sequence that encodes a human IL-15 protein is located at the mouse IL-15 genomic locus, the regulatory sequences operably linked to the human IL-15 coding sequence are endogenous, or native, to the mouse genome, i.e., they were present in the mouse genome prior to integration of human nucleic acid sequences.

In some instances, the humanized IL-15 non-human animal, e.g., mouse, is generated by the random integration, or insertion, of a human nucleic acid sequence encoding a human IL-15 protein (including fragments as described above), i.e., a “human IL-15 nucleic acid sequence”, or “human IL-15 sequence”, into the genome. Typically, in such embodiments, the location of the nucleic acid sequence encoding a human IL-15 protein in the genome is unknown. In other instances, the humanized IL-15 non-human animal is generated by the targeted integration, or insertion, of human IL-15 nucleic acid sequence into the genome, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a humanized IL-15 mouse including a nucleic acid sequence that encodes a human IL-15 protein created by targeting human IL-15 nucleic acid sequence to the mouse IL-15 locus, human IL-15 nucleic acid sequence may replace some or all of the mouse sequence, e.g. exons and/or introns, at the IL-15 locus. In some such instances, a human IL-15 nucleic acid sequence is integrated into the mouse IL-15 locus such that expression of the human IL-15 sequence is regulated by the native, or endogenous, regulatory sequences at the mouse IL-15 locus. In other words, the regulatory sequence(s) to which the nucleic acid sequence encoding a human IL-15 protein is operably linked are the native IL-15 regulatory sequences at the mouse IL-15 locus.

In some instances, the integration of a human IL-15 sequence does not affect the transcription of the gene into which the human IL-15 sequence has integrated. For example, if the human IL-15 sequence integrates into a coding sequence as an intein, or the human IL-15 sequence includes a 2A peptide, the human IL-15 sequence will be transcribed and translated simultaneously with the gene into which the human IL-15 sequence has integrated. In other instances, the integration of the human IL-15 sequence interrupts the transcription of the gene into which the human IL-15 sequence has integrated. For example, upon integration of the human IL-15 sequence by homologous recombination, some or all of the coding sequence at the integration locus may be removed, such that the human IL-15 sequence is transcribed instead. In some such instances, the integration of a human IL-15 sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele is indistinguishable from a deletion of the entire locus.

In some instances, the humanized IL-15 non-human animal, e.g., mouse, includes one copy of the nucleic acid sequence encoding a human IL-15 protein. For example, the non-human animal may be heterozygous for the nucleic acid sequence. In other words, one allele at a locus will include the nucleic acid sequence, while the other will be the endogenous allele. For example, as discussed above, in some instances, a human IL-15 nucleic acid sequence is integrated into the non-human animal, e.g., mouse, IL-15 locus such that it creates a null allele for the non-human animal IL-15. In some such embodiments, the humanized IL-15 non-human animal may be heterozygous for the nucleic acid sequence encoding human IL-15, i.e., the humanized IL-15 non-human animal includes one null allele for the non-human animal IL-15 (the allele including the nucleic acid sequence) and one endogenous IL-15 allele (wild-type or otherwise). In other words, the non-human animal is an IL-15^{h/m} non-human animal, where “h” represents the allele including the human sequence and “m” represents the endogenous allele. In other instances, the humanized IL-15 includes two copies of the nucleic acid sequence encoding a human IL-15 protein. For example, the non-human animal, e.g., mouse, may be homozygous for the nucleic acid sequence, i.e., both alleles for a locus in the diploid genome will include the nucleic acid sequence, i.e., the humanized IL-15 non-human animal includes two null alleles for the non-human animal IL-15 (the allele including the nucleic acid sequence). In other words, the non-human animal is an IL-15 hh non-human animal.

### Humanized SIRPα-IL-15 Non-Human Animals

By crossing humanized IL-15 non-human animals as described above with humanized SIRPα non-human animals of the same species as described above, genetically modified non-human animals expressing both human SIRPα and human IL-15 can be produced. In some embodiments, such genetically modified non-human animals are deficient for an endogenous immune system e.g., immunocompromised animals, e.g., as a result of a null allele for one or both of Rag2 and IL2rg. For example, in some embodiments a non-human animal according to the present disclosure is Rag2^{-/-} and/or IL2rg^{-/-} (or Rag2^{-/-} and/or IL2rg^{Y/-} where the IL2rg gene is located on the X chromosome as in mouse). In some embodiments, a genetically modified non-human animal, e.g., mouse, is provided wherein the genetically modified non-human animal, e.g., mouse is SIRPα^{h/m} IL-15^{h/m} Rag2^{-/-} IL2rg^{Y/-}, SIRPα^{h/h} IL-15^{h/m} Rag2^{-/-} IL2rg^{Y/-}, or SIRPα^{h/m} IL-15^{h/h} Rag2^{-/-} IL2rg^{Y/-}.

In some embodiments, a genetically modified non-human animal, e.g., mouse, is provided which includes a nucleic acid sequence incorporated into the genome of the genetically modified non-human animal, which sequence encodes a human SIRPα protein and is operably linked to a SIRPα gene promoter; and a nucleic acid sequence incorporated into the genome of the genetically modified non-human animal, which sequence encodes a human IL-15 protein and is operably linked to an IL-15 gene promoter, wherein the genetically modified non-human animal expresses the human SIRPα protein and the human IL-15 protein.

In some embodiments, the SIRPα gene promoter is an endogenous non-human SIRPα gene promoter. In some such embodiments, the SIRPα gene promoter is the endogenous non-human SIRPα gene promoter at the non-human animal SIRPα gene locus. In another embodiment, the SIRPα gene promoter is a human SIRPα promoter.

In some embodiments, the IL-15 gene promoter is an endogenous non-human IL-15 gene promoter. In some such embodiments, the IL-15 gene promoter is the endogenous non-human IL-15 gene promoter at the non-human animal IL-15 gene locus. In another embodiment, the IL-15 promoter is a human IL-15 promoter.

In some embodiments, a genetically modified non-human animal as described herein expresses human IL-15 mRNA in the liver, lung, bone marrow (BM), small intestine (SI) and colon.

In some embodiments, a genetically modified non-human animal, e.g., mouse, expressing both human SIRPα and human IL-15 as described herein exhibits a higher percentage and number of human T cells and NK cells than a genetically modified non-human animal, e.g., mouse, expressing only human SIRPα, following engraftment with human hematopoietic cells, e.g., CD45+ cells. In some embodiments, a genetically modified non-human animal, e.g., mouse, expressing both human SIRPα and human IL-15 as described herein exhibits a higher percentage and number of NK cells in blood and spleen. In some embodiments a genetically modified non-human animal, e.g., mouse, expressing both human SIRPα and human IL-15 as described herein includes both human NK cell subsets, CD56^{bright}CD16⁻ and CD56^{dim}CD16⁺, in the blood, spleen and liver, following engraftment with human hematopoietic cells, e.g., CD45+ cells. In some embodiments, a genetically modified non-human animal, e.g., mouse, expressing both human SIRPα and human IL-15 as described herein exhibits similar distribution of CD16+ versus CD16− NK cells in blood as the distribution of CD16+ versus CD16− NK cells in PBMCs obtained from human subjects.

In some embodiments a genetically modified non-human animal, e.g., mouse, expressing both human SIRPα and human IL-15 as described herein includes NK cells in the liver of the genetically modified non-human animal which exhibit a higher expression level of CD16 and CD56, indicating increased NK cell maturation, relative to a genetically modified non-human animal, e.g., mouse, expressing only human SIRPα, following engraftment with human hematopoietic cells, e.g., CD45+ cells.

In some embodiments a genetically modified non-human animal, e.g., mouse, expressing both human SIRPα and human IL-15 as described herein, and engrafted with human hematopoietic cells, e.g., CD45+ cells, includes NK cells in the spleen which exhibit a distinct expression level of killer inhibitory receptors, with the CD56^{dim}CD16⁺ NK cell population including the higher percentage of CD158− expressing cells, similar to what is found for NK cell subsets in the blood of humans.

In some embodiments a genetically modified non-human animal, e.g., mouse, expressing both human SIRPα and human IL-15 as described herein, and engrafted with human hematopoietic cells, e.g., CD45+ cells, exhibits a higher frequency of human CD45+ and CD8+ T cells in the intraepithelial lymphocyte population relative to a genetically modified non-human animal, e.g., mouse, expressing only human SIRPα. In some embodiments, a genetically modified non-human animal, e.g., mouse, expressing both human SIRPα and human IL-15 as described herein, and engrafted with human hematopoietic cells, exhibits comparable CD16+ versus CD16− NK cell distribution in IELs, and more CD16+ than CD16− NK cells in blood and spleen, which is reflective of normal human physiology.

In some embodiments a genetically modified non-human animal, e.g., mouse, expressing both human SIRPα and human IL-15 as described herein, and engrafted with human hematopoietic cells, e.g., CD45+ cells, exhibits an increased number of human T cells in the lung relative to a genetically modified non-human animal, e.g., mouse, expressing only human SIRPα. In some such embodiments, such a genetically modified non-human animal, e.g., mouse, exhibits a higher level of expression of CD69 on human CD8+ T cells in the lung relative to a genetically modified non-human animal, e.g., mouse, expressing only human SIRPα.

In some embodiments a genetically modified non-human animal, e.g., mouse, expressing both human SIRPα and human IL-15 as described herein, and engrafted with human hematopoietic cells, e.g., CD45+ cells, exhibits an increased level of CD69 expression on human CD8+ T cells in the liver relative to a genetically modified non-human animal, e.g., mouse, expressing only human SIRPα.

In some embodiments, a genetically modified non-human animal, e.g., mouse, expressing both human SIRPα and human IL-15 as described herein, and engrafted with human hematopoietic cells, exhibits discernable Peyer's Patches which are predominantly human CD45+.

Any non-human mammal animal may be genetically modified according to the subject disclosure. Nonlimiting examples include laboratory animals, domestic animals, livestock, etc., e.g., species such as murine, rodent, canine, feline, porcine, equine, bovine, ovine, non-human primates, etc.; for example, mice, rats, rabbits, hamsters, guinea pigs, cattle, pigs, sheep, goats and other transgenic animal species, particularly-mammalian species, as known in the art. In other embodiments, the non-human animal may be a bird, e.g., of Galliformes order, such as a chicken, a turkey, a quail, a pheasant, or a partridge; e.g., of Anseriformes order, such as a duck, a goose, or a swan, e.g., of Columbiformes order, such as a pigeon or a dove. In various embodiments, the subject genetically modified animal is a mouse, a rat or a rabbit.

In some embodiments, the non-human animal is a mammal. In some such embodiments, the non-human animal is a small mammal, e.g., of the superfamily Dipodoidea or Muroidea. In one embodiment, the genetically modified animal is a rodent. In one embodiment, the rodent is selected from a mouse, a rat, and a hamster. In one embodiment, the rodent is selected from the superfamily Muroidea. In one embodiment, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, white-tailed rats, Malagasy rats and mice), Platacanthomyidae (e.g., spiny dormice), and Spalacidae (e.g., mole rates, bamboo rats, and zokors). In a specific embodiment, the genetically modified rodent is selected from a true mouse or rat (family Muridae), a gerbil, a spiny mouse, and a crested rat.

In one embodiment, the subject genetically modified non-human animal is a rat. In one such embodiment, the rat is selected from a Wistar rat, an LEA strain, a Sprague Dawley strain, a Fischer strain, F344, F6, and Dark Agouti. In another embodiment, the rat strain is a mix of two or more strains selected from the group consisting of Wistar, LEA, Sprague Dawley, Fischer, F344, F6, and Dark Agouti.

In another embodiment, the subject genetically modified non-human animal is a mouse, e.g. a mouse of a C57BL strain (e.g. C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, C57BL/Ola, etc.); a mouse of the 129 strain (e.g. 129P1, 129P2, 129P3,129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2); a mouse of the BALB strain; e.g., BALB/c; and the like. See, e.g., Festing et al. (1999) Mammalian Genome 10:836, see also, Auerbach et al (2000) Establishment and Chimera Analysis of 129/SvEv− and C57BL/6-Derived Mouse Embryonic Stem Cell Lines). In another embodiment, a mouse is a mix of the aforementioned strains.

In some embodiments, the subject genetically modified non-human animal is also immunodeficient. “Immunodeficient,” includes deficiencies in one or more aspects of an animal's native, or endogenous, immune system, e.g. the animal is deficient for one or more types of functioning host immune cells, e.g. deficient for non-human B cell number and/or function, non-human T cell number and/or function, non-human NK cell number and/or function, etc.

One method to achieve immunodeficiency in the subject animals is sublethal irradiation. For example, newborn genetically modified mouse pups can be irradated sublethally, e.g., 2×200 cGy with a four hour interval. Alternatively, immunodeficiency may be achieved by any one of a number of gene mutations known in the art, any of which may be bred either alone or in combination into the subject genetically modified non-human animals of the present disclosure or which may be used as the source of stem cells into which the genetic modifications of the subject disclosure may be introduced. Non-limiting examples include X-linked SCID, associated with IL2rg gene mutations and characterized by the lymphocyte phenotype T(−) B(+) NK(−); autosomal recessive SCID associated with Jak3 gene mutations and characterized by the lymphocyte phenotype T(−) B(+) NK(−); ADA gene mutations characterized by the lymphocyte phenotype T(−) B(−) NK(−); IL-7R alpha-chain mutations characterized by the lymphocyte phenotype T(−) B(+) NK(+); CD3 delta or epsilon mutations characterized by the lymphocyte phenotype T(−) B(+) NK(+); RAG1 and RAG2 mutations characterized by the lymphocyte phenotype T(−) B(−) NK(+); Artemis gene mutations characterized by the lymphocyte phenotype T(−) B(−) NK(+), CD45 gene mutations characterized by the lymphocyte phenotype T(−) B(+) NK(+); and Prkdcscid mutations characterized by the lymphocyte phenotype T(−), B(−). As such, in some embodiments, the genetically modified immunodeficient non-human animal has one or more deficiencies selected from an IL2 receptor gamma chain (II2rg^{y/-}) deficiency, a Jak3 deficiency, an ADA deficiency, an IL7R deficiency, a CD3 deficiency, a RAG1 and/or RAG2 deficiency, an Artemis deficiency, a CD45 deficiency, and a Prkdc deficiency. These and other animal models of immunodeficiency will be known to the ordinarily skilled artisan, any of which may be used to generate immunodeficient animals of the present disclosure.

In some embodiments, genetically modified non-human animals in accordance with the invention find use as recipients of human hematopoietic cells that are capable of developing human immune cells from engrafted human hematopoietic cells. As such, in some aspects of the invention, the subject genetically modified animal is a genetically modified, immunodeficient, non-human animal that is engrafted with human hematopoietic cells.

### Engraftment of Humanized SIRPα-IL-15 Non-Human Animals

As discussed above, in some aspects of the invention, the humanized SIRPα-IL-15 non-human animal, e.g., mouse, e.g., a Rag2^{-/-} IL2rg^{Y/-} hSIRPα hIL-15 mouse, or a sublethally irradiated hSIRPα hIL-15 mouse, is engrafted, or transplanted, with cells. Cells may be mitotic cells or post-mitotic cells, and include such cells of interest as pluripotent stem cells, e.g., ES cells, iPS cells, and embryonic germ cells; and somatic cells, e.g., fibroblasts, hematopoietic cells, neurons, muscle cells, bone cells, vascular endothelial cells, gut cells, and the like, and their lineage-restricted progenitors and precursors. Cell populations of particular interest include those that include hematopoietic stem or progenitor cells, which will contribute to or reconstitute the hematopoietic system of the humanized SIRPα-IL-15 non-human animal, for example, peripheral blood leukocytes, fetal liver cells, fetal bone, fetal thymus, fetal lymph nodes, vascularized skin, artery segments, and purified hematopoietic stem cells, e.g., mobilized HSCs or cord blood HSCs.

Any source of human hematopoietic cells, human hematopoietic stem cells (HSCs) and/or hematopoietic stem progenitor cells (HSPC) as known in the art or described herein may be transplanted into the genetically modified immunodeficient non-human animals of the present disclosure. One suitable source of human hematopoietic cells known in the art is human umbilical cord blood cells, in particular CD34-positive (CD34⁺) cells. Another source of human hematopoietic cells is human fetal liver. Another source is human bone marrow. Also encompassed are induced pluripotent stem cells (iPSC) and induced hematopoietic stem cells (iHSC) produced by the de-differentiation of somatic cells, e.g., by methods known in the art.

Cells may be from any mammalian species, e.g., murine, rodent, canine, feline, equine, bovine, ovine, primate, human, etc. Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e., splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

If the cells are primary cells, they may be harvested from an individual by any convenient method. For example, cells, e.g., blood cells, e.g., leukocytes, may be harvested by apheresis, leukocytapheresis, density gradient separation, etc. As another example, cells, e.g., skin, muscle, bone marrow, spleen, liver, pancreas, lung, intestine, stomach tissue, etc. may be harvested by biopsy. An appropriate solution may be used for dispersion or suspension of the harvested cells. Such solution will generally be a balanced salt solution, e.g., normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

In some instances, a heterogeneous population of cells will be transplanted into the humanized non-human animal, e.g., mouse. In other instances, a population of cells that is enriched for a particular type of cell, e.g., a progenitor cell, e.g., a hematopoietic progenitor cell, will be engrafted into the humanized non-human animal, e.g., mouse. Enrichment of a cell population of interest may be by any convenient separation technique. For example, the cells of interest may be enriched by culturing methods. In such culturing methods, particular growth factors and nutrients are typically added to a culture that promotes the survival and/or proliferation of one cell population over others. Other culture conditions that affect survival and/or proliferation include growth on adherent or non-adherent substrates, culturing for particular lengths of time, etc. Such culture conditions are well known in the art. As another example, cells of interest may be enriched for by separation the cells of interest from the initial population by affinity separation techniques. Techniques for affinity separation may include magnetic separation using magnetic beads coated with an affinity reagent, affinity chromatography, “panning” with an affinity reagent attached to a solid matrix, e.g., plate, cytotoxic agents joined to an affinity reagent or used in conjunction with an affinity reagent, e.g., complement and cytotoxins, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the cells of interest.

For example, using affinity separation techniques, cells that are not the cells of interest for transplantation may be depleted from the population by contacting the population with affinity reagents that specifically recognize and selectively bind markers that are not expressed on the cells of interest. For example, to enrich for a population of hematopoietic progenitor cells, one might deplete cells expressing mature hematopoietic cell markers. Additionally or alternatively, positive selection and separation may be performed using by contacting the population with affinity reagents that specifically recognize and selectively bind markers associated with hematopoietic progenitor cells, e.g. CD34, CD133, etc. By “selectively bind” is meant that the molecule binds preferentially to the target of interest or binds with greater affinity to the target than to other molecules. For example, an antibody will bind to a molecule including an epitope for which it is specific and not to unrelated epitopes. In some embodiments, the affinity reagent may be an antibody, i.e. an antibody that is specific for CD34, CD133, etc. In some embodiments, the affinity reagent may be a specific receptor or ligand for CD34, CD133, etc., e.g., a peptide ligand and receptor; effector and receptor molecules, a T-cell receptor specific for CD34, CD133, etc., and the like. In some embodiments, multiple affinity reagents specific for the marker of interest may be used.

Antibodies and T cell receptors that find use as affinity reagents may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art. Of particular interest is the use of labeled antibodies as affinity reagents. Conveniently, these antibodies are conjugated with a label for use in separation. Labels include magnetic beads, which allow for direct separation; biotin, which can be removed with avidin or streptavidin bound to a support; fluorochromes, which can be used with a fluorescence activated cell sorter; or the like, to allow for ease of separation of the particular cell type. Fluorochromes that find use include phycobiliproteins, e.g., phycoerythrin and allophycocyanins, fluorescein and Texas red. Frequently each antibody is labeled with a different fluorochrome, to permit independent sorting for each marker.

The initial population of cells are contacted with the affinity reagent(s) and incubated for a period of time sufficient to bind the available cell surface antigens. The incubation will usually be at least about 5 minutes and usually less than about 60 minutes. It is desirable to have a sufficient concentration of antibodies in the reaction mixture, such that the efficiency of the separation is not limited by lack of antibody. The appropriate concentration is determined by titration, but will typically be a dilution of antibody into the volume of the cell suspension that is about 1:50 (i.e., 1 part antibody to 50 parts reaction volume), about 1:100, about 1:150, about 1:200, about 1:250, about 1:500, about 1:1000, about 1:2000, or about 1:5000. The medium in which the cells are suspended will be any medium that maintains the viability of the cells. A preferred medium is phosphate buffered saline containing from 0.1 to 0.5% BSA or 1-4% goat serum. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, goat serum etc.

The cells in the contacted population that become labeled by the affinity reagent are selected for by any convenient affinity separation technique, e.g., as described above or as known in the art. Following separation, the separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

Compositions highly enriched for a cell type of interest, e.g., hematopoietic cells, are achieved in this manner. The cells will be about 70%, about 75%, about 80%, about 85% about 90% or more of the cell composition, about 95% or more of the enriched cell composition, and will preferably be about 95% or more of the enriched cell composition. In other words, the composition will be a substantially pure composition of cells of interest.

The cells to be transplanted into the humanized SIRPα-IL-15 non-human animals, e.g., mice, be they a heterogeneous population of cells or an enriched population of cells, may be transplanted immediately. Alternatively, the cells may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells. Additionally or alternatively, the cells may be cultured in vitro under various culture conditions. Culture medium may be liquid or semi-solid, e.g. containing agar, methylcellulose, etc. The cell population may be conveniently suspended in an appropriate nutrient medium, such as Iscove's modified DMEM or RPMI-1640, normally supplemented with fetal calf serum (about 5-10%), L-glutamine, a thiol, particularly 2-mercaptoethanol, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

The cells may be genetically modified prior to transplanting to the SIRPα-IL-15 non-human animals, e.g., mice, e.g., to provide a selectable or traceable marker, to induce a genetic defect in the cells (e.g., for disease modeling), to repair a genetic defect or ectopically express a gene in the cells (e.g., to determine if such modifications will impact the course of a disease), etc. Cells may be genetically modified by transfection or transduction with a suitable vector, homologous recombination, or other appropriate technique, so that they express a gene of interest, or with an antisense mRNA, siRNA or ribozymes to block expression of an undesired gene. Various techniques are known in the art for the introduction of nucleic acids into target cells. To prove that one has genetically modified the cells, various techniques may be employed. The genome of the cells may be restricted and used with or without amplification. The polymerase chain reaction; gel electrophoresis; restriction analysis; Southern, Northern, and Western blots; sequencing; or the like, may all be employed. General methods in molecular and cellular biochemistry for these and other purposes disclosed in this application can be found in such standard textbooks as Molecular Cloning: A Laboratory Manual, 3rd Ed. (Sambrook et al., Cold Spring Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

The cells may be transplanted in the humanized SIRPα-IL-15 non-human animals, e.g., mice, by any convenient method, including, for example, intra-hepatic injection, tail-vein injection, retro-orbital injection, and the like. Typically, about 0.5×10⁵-2×10⁶ pluripotent or progenitor cells are transplanted, e.g. about 1×10⁵-1×10⁶ cells, or about 2×10⁵-5×10⁵ cells. In some instances, the non-human animal, e.g., mouse, is sublethally irradiated prior to transplanting the human cells. In other words, the non-human animal, e.g., mouse, is exposed to a sublethal dose of radiation, e.g., as well-known in the art. The engrafted humanized SIRPα-IL-15 non-human animals, e.g., mice, are then maintained under laboratory animal husbandry conditions for at least 1 week, e.g., 1 week or more, or two weeks or more, sometimes 4 weeks or more, and in some instances 6 weeks or more, such as 10 weeks or more or 15 weeks or more, to allow sufficient reconstitution of the immune system with the engrafted cells.

The humanized SIRPα-IL-15 non-human animals, e.g., mice, and humanized SIRPα-IL-15 non-human animals, e.g., mice, engrafted with human hematopoietic cells, e.g., engrafted Rag2^{-/-}IL2rg^{Y/-} hSIRPα hIL-15 mice, and optionally other genetic modifications are useful in many applications. For example, these non-human animals, e.g., mice, provide a useful system for modeling human immune diseases and human pathogens. For example, the subject non-human animals, e.g., mice, are useful for modeling, for example, human T cell and/or natural killer (NK) cell development and function; human pathogen infection of specific tissues and/or cells, e.g., human pathogen infection of the gut or lungs, and/or human pathogen infection of or response to human T cells and/or NK cells. Such non-human animals also find use in in vivo screens for agents that inhibit infection by a pathogen, e.g., a pathogen that affects (e.g., by infecting) a specific tissue or cell type, e.g., a human pathogen of the gut or lungs, e.g., a human pathogen that activates, induces and/or targets T cells and/or NK cells; in in vivo screens for agents that modulate the development and/or function of human T cells and/or NK cells, e.g. in a healthy or a diseased state; in in vivo screens for agents that are toxic to human T cells and/or NK cells; in in vivo screens for agents that prevent against, mitigate, or reverse the toxic effects of toxic agents on human T cells and/or NK cells; in in vivo screens of candidate T cell-inducing vaccines; and in in vivo and in vitro screens for agents that inhibit tumor growth and/or infection by activating NK cell-mediated antibody dependent cellular cytotoxicity (ADCC) processes.

The present disclosure provides unexpected results demonstrating that humanized SIRPα-IL-15 non-human animals, e.g., mice, engrafted with human hematopoietic cells, e.g., engrafted Rag2^{-/-}IL2rg^{Y/-} hSIRPα hIL-15 mice, develop tissue-resident lymphocytes, e.g., intraepithelial lymphocytes, in the gut and lung. Accordingly, the present disclosure provides previously unavailable animal models which enable the monitoring and testing of such tissue-resident lymphocytes. Such animal models are particularly useful in modeling the immune response of tissue-resident lymphocytes, e.g., T cells and NK cells, to human pathogens which affect (e.g., by infecting) the gut and/or lung and for screening therapeutics and vaccines which target such pathogens and/or induce or improve a tissue-resident lymphocyte response. In addition, the presence of these tissue-resident lymphocytes also allows for modeling of human immune cell driven autoimmune diseases that affect the gastrointestinal tract such as celiac diseases and IBD.

Accordingly, in some embodiments, the present disclosure provides an in vivo model, including a genetically modified non-human animal including a nucleic acid sequence incorporated into the genome of the genetically modified non-human animal, which sequence encodes a human SIRPα protein and is operably linked to a SIRPα gene promoter. The genetically modified non-human animal also includes a nucleic acid sequence incorporated into the genome of the genetically modified non-human animal, which sequence encodes a human IL-15 protein and is operably linked to an IL-15 gene promoter. Finally, the genetically modified non-human animal includes an engraftment of human hematopoietic cells, wherein the genetically modified non-human animal (i) expresses the human SIRPα protein and the human IL-15 protein, and (ii) includes human tissue-resident lymphocytes, e.g., intraepithelial lymphocytes (IELs), in the gut of the genetically modified non-human. In some such embodiments, the genetically modified non-human animal is infected with a human pathogen, e.g., a human pathogen which affects (e.g., by infecting) the gut.

Human pathogens which can affect (e.g., by infecting) the gut include, but are not limited to, Campylobacter jejuni, Clostridium difficile, Enterococcus faecalis, Enterococcus faecium, Escherichia coli, Human Rotavirus, Listeria monocytogenes, Norwalk Virus, Salmonella enterica, Shigella flexneri, Shigella sonnei, Shigella dysenteriae, Yersinia pestis, Yersinia enterocolitica, and Helicobacter pylori.

In other embodiments, the present disclosure provides an in vivo model, including a genetically modified non-human animal including a nucleic acid sequence incorporated into the genome of the genetically modified non-human animal, which sequence encodes a human SIRPα protein and is operably linked to a SIRPα gene promoter. The genetically modified non-human animal also includes a nucleic acid sequence incorporated into the genome of the genetically modified non-human animal, which sequence encodes a human IL-15 protein and is operably linked to an IL-15 gene promoter. Finally, the genetically modified non-human animal includes an engraftment of human hematopoietic cells, wherein the genetically modified non-human animal (i) expresses the human SIRPα protein and the human IL-15 protein, and (ii) includes human tissue-resident lymphocytes, e.g., intraepithelial lymphocytes (IELs), in the lung of the genetically modified non-human. In some such embodiments, the genetically modified non-human animal is infected with a human pathogen, e.g., a human pathogen which affects (e.g., by infecting) the lung.

Human pathogens which can affect (e.g., by infecting) the lung include, but are not limited to, Streptococcus pyogenes, Haemophilus influenza, Corynebacterium diphtheria, SARS coronavirus, Bordetella pertussis, Moraxella catarrhalis, Influenza virus (A, B, C), Coronavirus, Adenovirus, Respiratory Syncytial Virus, Parainfluenza virus, Mumps virus, Streptococcus pneumoniae, Staphylococcus aureus, Legionella pneumophila, Klebsiella pneumoniae, Pseudomonas aeruginosa, Mycoplasma pneumonia, Mycobacterium tuberculosis, Chlamydia Pneumoniae, Blastomyces dermatitidis, Cryptococcus neoformans, and Aspergillus fumigatus.

New therapeutics, new vaccines, and new ways of testing efficacy of therapeutics and vaccines are needed. A non-human animal, e.g., mouse, which supports efficient human T and NK cell engraftment, for example, would be useful to identify new therapeutics and new vaccines, particularly for a human pathogen which infects human T cells and/or NK cells. New therapeutics and new vaccines could be tested in such a non-human animal, e.g., mouse, by, e.g., determining the amount of a human pathogen, e.g., a virus, in the non-human animal (in blood or a given tissue) in response to treatment with a putative anti-viral agent, or by inoculating the mouse with a putative vaccine followed by exposure to an infective administration of a human pathogen, e.g., HIV, and observing any change in infectivity due to inoculation by the putative vaccine as compared to a control not inoculated with the vaccine but infected with HIV.

Such non-human animal, e.g., mouse, models of pathogen infection are useful in research, e.g., to better understand the progression of human infection. Such mouse models of infection are also useful in drug discovery, e.g. to identify candidate agents that protect against or treat infection.

Engrafted genetically modified animals of the present disclosure find use in screening candidate agents to identify those that will treat infections by human pathogens, e.g., human pathogens that target human T and/or NK cells. The terms “treat”, “treatment”, “treating” and the like are used herein to generally include obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein include any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease.

The terms “individual,” “subject,” “host,” and “patient,” are used interchangeably herein and include any mammalian subject for whom diagnosis, treatment, or therapy is desired, particularly humans.

Humanized SIRPα-IL-15 non-human animals, e.g., mice, engrafted with human hematopoietic cells provide a useful system for screening candidate agents for other desired activities in vivo as well, for example, for agents that are able to modulate (i.e., promote or suppress) development and/or activity of human T cells and NK cells, e.g., in a healthy or a diseased state, e.g., to identify novel therapeutics and/or develop a better understanding of the molecular basis of the development and function of the immune system; for agents that are toxic to T cells and/or NK cells and progenitors thereof; and for agents that prevent against, mitigate, or reverse the toxic effects of toxic agents on T cells, NK cells, and progenitors thereof; for antibodies or antigen-binding proteins that mediate NK cell dependent ADCC processes, etc. As yet another example, the genetically modified mice described herein provide a useful system for predicting the responsiveness of an individual to a disease therapy, e.g., by providing an in vivo platform for screening the responsiveness of an individual's immune system to an agent, e.g., a therapeutic agent, to predict the responsiveness of an individual to that agent.

In screening assays for biologically active agents, humanized SIRPα-IL-15 non-human animals, e.g., mice, e.g., engrafted Rag2^{-/-}IL2rg^{Y/-} hSIRPα hIL-15 mice, that have been engrafted with human hematopoietic cells and in some instances, infected with human pathogens, or cells to be engrafted into a humanized SIRPα-IL-15 non-human animal, e.g., mouse, are contacted with a candidate agent of interest and the effect of the candidate agent is assessed by monitoring one or more output parameters. These output parameters may be reflective of the viability of the cells, e.g. the total number of hematopoietic cells or the number of cells of a particular hematopoietic cell type, or of the apoptotic state of the cells, e.g. the amount of DNA fragmentation, the amount of cell blebbing, the amount of phosphatidylserine on the cell surface, and the like by methods that are well known in the art. Alternatively or additionally, the output parameters may be reflective of the differentiation capacity of the cells, e.g. the proportions of differentiated cells and differentiated cell types, e.g., T cells and/or NK cells. Alternatively or additionally, the output parameters may be reflective of the function of the cells, e.g. the cytokines and chemokines produced by the cells, the ability of the cells to home to and extravasate to a site of challenge, the ability of the cells to modulate, i.e. promote or suppress, the activity of other cells in vitro or in vivo, etc. Other output parameters may be reflective of the extent of pathogen infection in the animal, e.g., the titer of pathogen in the non-human animal, e.g., mouse, etc.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Candidate agents of interest for screening include known and unknown compounds that encompass numerous chemical classes, primarily organic molecules, which may include organometallic molecules, inorganic molecules, genetic sequences, vaccines, antibiotics or other agents suspected of having antibiotic properties, peptides, polypeptides, antibodies, antigen-binding proteins, agents that have been approved pharmaceutical for use in a human, etc. An important aspect of the invention is to evaluate candidate drugs, including toxicity testing; and the like.

Candidate agents include organic molecules including functional groups necessary for structural interactions, particularly hydrogen bonding, and typically include at least an amine, carbonyl, hydroxyl or carboxyl group, frequently at least two of the functional chemical groups. The candidate agents often include cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Candidate agents of interest for screening also include nucleic acids, for example, nucleic acids that encode siRNA, shRNA, antisense molecules, or miRNA, or nucleic acids that encode polypeptides. Many vectors useful for transferring nucleic acids into target cells are available. The vectors may be maintained episomally, e.g., as plasmids, minicircle DNAs, virus-derived vectors such cytomegalovirus, adenovirus, etc., or they may be integrated into the target cell genome, through homologous recombination or random integration, e.g., retrovirus derived vectors such as MMLV, HIV-1, ALV, etc. Vectors may be provided directly to the subject cells. In other words, the pluripotent cells are contacted with vectors including the nucleic acid of interest such that the vectors are taken up by the cells.

Methods for contacting cells, e.g., cells in culture or cells in a non-human animal, e.g., mouse, with nucleic acid vectors, such as electroporation, calcium chloride transfection, and lipofection, are well known in the art. Alternatively, the nucleic acid of interest may be provided to the cells via a virus. In other words, the cells are contacted with viral particles including the nucleic acid of interest. Retroviruses, for example, lentiviruses, are particularly suitable to the method of the invention. Commonly used retroviral vectors are “defective”, i.e., unable to produce viral proteins required for productive infection. Rather, replication of the vector requires growth in a packaging cell line. To generate viral particles including nucleic acids of interest, the retroviral nucleic acids including the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g., MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g., AKR env, are capable of infecting most mammalian cell types, except murine cells. The appropriate packaging cell line may be used to ensure that the cells of interest—in some instance, the engrafted cells, in some instance, the cells of the host, i.e., the humanized SIRPα-IL-15—are targeted by the packaged viral particles.

Vectors used for providing nucleic acid of interest to the subject cells will typically include suitable promoters for driving the expression, that is, transcriptional activation, of the nucleic acid of interest. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing reprogramming factors to the subject cells may include genes that must later be removed, e.g., using a recombinase system such as Cre/Lox, or the cells that express them destroyed, e.g., by including genes that allow selective toxicity such as herpesvirus TK, bcl-xs, etc.

Candidate agents of interest for screening also include polypeptides. Such polypeptides may optionally be fused to a polypeptide domain that increases solubility of the product. The domain may be linked to the polypeptide through a defined protease cleavage site, e.g., a TEV sequence, which is cleaved by TEV protease. The linker may also include one or more flexible sequences, e.g., from 1 to 10 glycine residues. In some embodiments, the cleavage of the fusion protein is performed in a buffer that maintains solubility of the product, e.g., in the presence of from 0.5 to 2 M urea, in the presence of polypeptides and/or polynucleotides that increase solubility, and the like. Domains of interest include endosomolytic domains, e.g., influenza HA domain; and other polypeptides that aid in production, e.g., IF2 domain, GST domain, GRPE domain, and the like. Additionally or alternatively, such polypeptides may be formulated for improved stability. For example, the peptides may be PEGylated, where the polyethyleneoxy group provides for enhanced lifetime in the blood stream. The polypeptide may be fused to another polypeptide to provide for added functionality, e.g., to increase the in vivo stability. Generally such fusion partners are a stable plasma protein, which may, for example, extend the in vivo plasma half-life of the polypeptide when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the polypeptide also will be produced and employed as a multimer having substantially the same structure as the stable plasma protein precursor. These multimers will be homogeneous with respect to the polypeptide agent they include, or they may contain more than one polypeptide agent.

The candidate polypeptide agent may be produced from eukaryotic cells, or may be produced by prokaryotic cells. It may be further processed by unfolding, e.g., heat denaturation, DTT reduction, etc., and may be further refolded, using methods known in the art. Modifications of interest that do not alter primary sequence include chemical derivatization of polypeptides, e.g., acylation, acetylation, carboxylation, amidation, etc. Also included are modifications of glycosylation, e.g., those made by modifying the glycosylation patterns of a polypeptide during its synthesis and processing or in further processing steps; e.g., by exposing the polypeptide to enzymes which affect glycosylation, such as mammalian glycosylating or deglycosylating enzymes. Also embraced are sequences that have phosphorylated amino acid residues, e.g., phosphotyrosine, phosphoserine, or phosphothreonine. The polypeptides may have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. D-amino acids may be substituted for some or all of the amino acid residues.

The candidate polypeptide agent may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Alternatively, the candidate polypeptide agent may be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will include at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In some cases, the candidate polypeptide agents to be screened are antibodies or antigen-binding proteins. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured. Besides antibodies, antigen-binding proteins encompass polypeptides that are also designed to bind an antigen of interest and elicit a response, e.g., an immunological reaction. Antigen-binding fragments known in the art (including, e.g., Fab, Fab′ F(ab′)2, Fabc, and scFv) are also encompassed by the term “antigen-binding protein”. The terms “antibody” and “antigen-binding protein” also include one or more immunoglobulin chains or fragments that may be chemically conjugated to, or expressed as, fusion proteins with other proteins, single chain antibodies, and bispecific antibodies.

Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by administering the agent to at least one and usually a plurality of samples, sometimes in conjunction with samples lacking the agent. The change in parameters in response to the agent is measured, and the result evaluated by comparison to reference cultures, e.g. in the presence and absence of the agent, obtained with other agents, etc. In instances in which a screen is being performed to identify candidate agents that will prevent, mitigate or reverse the effects of a toxic agent, the screen is typically performed in the presence of the toxic agent, where the toxic agent is added at the time most appropriate to the results to be determined. For example, in cases in which the protective/preventative ability of the candidate agent is tested, the candidate agent may be added before the toxic agent, simultaneously with the candidate agent, or subsequent to treatment with the candidate agent. As another example, in cases in which the ability of the candidate agent to reverse the effects of a toxic agent is tested, the candidate agent may be added subsequent to treatment with the candidate agent. As mentioned above, in some instances, the sample is the humanized SIRPα-IL-15 non-human animal, e.g., mouse, that has been engrafted with cells, i.e., a candidate agent is provided to the humanized SIRPα-IL-15 non-human animal, e.g., mouse, that has been engrafted with cells. In some instances, the sample is the cells to be engrafted, i.e., the candidate agent is provided to cells prior to transplantation.

If the candidate agent is to be administered directly to the non-human animal, e.g., mouse, the agent may be administered by any of a number of well-known methods in the art for the administration of peptides, small molecules and nucleic acids. For example, the agent may be administered orally, mucosally, topically, intradermally, or by injection, e.g. intraperitoneal, subcutaneous, intramuscular, intravenous, or intracranial injection, and the like. The agent may be administered in a buffer, or it may be incorporated into any of a variety of formulations, e.g. by combination with appropriate pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semi-solid, liquid or gaseous forms, such as tablets, capsules, powders, granules, ointments, solutions, suppositories, injections, inhalants, gels, microspheres, and aerosols. The agent may be systemic after administration or may be localized by the use of regional administration, intramural administration, or use of an implant that acts to retain the active dose at the site of implantation. The active agent may be formulated for immediate activity or it may be formulated for sustained release. For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. A BBB disrupting agent can be co-administered with the agent when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of agents behind the BBB may be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. U.S. application No. 20070254842, incorporated here by reference); or by implanting a device upon which the agent has been reversably affixed (see e.g. U.S. application Nos. 20080081064 and 20090196903, incorporated herein by reference).

If the agent(s) are provided to cells prior to transplantation, the agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e., at zero concentration or below the level of detection of the agent or at or below the concentration of agent that does not give a detectable change in the phenotype.

An analysis of the response of cells in a humanized SIRPα-IL-15 non-human animal, e.g., mouse, to the candidate agent may be performed at any time following treatment with the agent. For example, the cells may be analyzed 1, 2, or 3 days, sometimes 4, 5, or 6 days, sometimes 8, 9, or 10 days, sometimes 14 days, sometimes 21 days, sometimes 28 days, sometimes 1 month or more after contact with the candidate agent, e.g., 2 months, 4 months, 6 months or more. In some embodiments, the analysis includes analysis at multiple time points. The selection of the time point(s) for analysis will be based upon the type of analysis to be performed, as will be readily understood by the ordinarily skilled artisan.

The analysis may include measuring any of the parameters described herein or known in the art for measuring cell viability, cell proliferation, cell identity, cell morphology, and cell function, particularly as they may pertain to cells of the immune system, e.g., T cells and/or NK cells. For example, flow cytometry may be used to determine the total number of hematopoietic cells or the number of cells of a particular hematopoietic cell type. Histochemistry or immunohistochemistry may be performed to determine the apoptotic state of the cells, e.g. terminal deoxynucleotidyl transferase dUTP nick end labeling (TUNEL) to measure DNA fragmentation, or immunohistochemistry to detect Annexin V binding to phosphatidylserine on the cell surface. Flow cytometry may also be employed to assess the proportions of differentiated cells and differentiated cell types, e.g., to determine the ability of hematopoietic cells to differentiate in the presence of agent. ELISAs, Westerns, and Northern blots may be performed to determine the levels of cytokines, chemokines, immunoglobulins, etc., expressed in the engrafted humanized SIRPα-IL-15 non-human animal, e.g., mouse, e.g. to assess the function of the engrafted cells. In vivo assays to test the function of immune cells, as well as assays relevant to particular diseases or disorders of interest such as diabetes, autoimmune disease, graft v. host disease, AMD, etc., may also be performed. See, e.g. Current Protocols in Immunology (Richard Coico, ed. John Wiley & Sons, Inc. 2012) and Immunology Methods Manual (I. Lefkovits ed., Academic Press 1997), the disclosures of which are incorporated herein by reference.

So, for example, a method is provided for determining the effect of an agent on a human pathogen, including exposing an engrafted humanized SIRPα-IL-15 non-human animal, e.g., mouse, e.g., an engrafted Rag2^{-/-}IL2rg^{Y/-} hSIRPα hIL-15 mouse, to an effective amount of a human pathogen, the effective amount of a pathogen being the amount of pathogen required to produce an infection in the mouse; allowing the pathogen to infect the mouse; measuring a parameter of the infection over time in the presence of the agent; and comparing that measurement to the measurement from an engrafted humanized SIRPα-IL-15 non-human animal, e.g., mouse, not exposed to the agent. The agent is determined to be an antipathogenic agent if it reduces the amount of the agent in blood or a tissue of the non-human animal, e.g., mouse, by at least half following a single administration or two or more administrations of the agent over a selected period of time.

As another example, a method is provided for determining if a pathogen isolate or strain of interest is drug resistant, e.g. multidrug resistant. In these methods, an engrafted humanized SIRPα-IL-15 non-human animal, e.g., mouse, e.g., an engrafted Rag2^{-/-} IL2rg^{Y/-} hSIRPα hIL-15 mouse, is exposed to an effective amount of a human pathogen isolate or strain of interest, the effective amount of the pathogen being the amount of pathogen required to produce an infection in the non-human animal, e.g., mouse; the pathogen is allowed to infect the non-human animal; a parameter of the infection, e.g., the titer of the isolate or strain of interest in the blood or tissue of the non-human animal, the ability of the isolate or strain of interest to maintain an infection in the non-human animal, or the ability of the isolate or strain of interest to reproduce in the non-human animal at a point in time after administration of the drug, is measured in the presence of the drug; and that measurement is compared to the measurement from an engrafted humanized SIRPα-IL-15 non-human animal, e.g., mouse infected with pathogen not exposed to the agent. Examples of drugs of interest include amoxicillin, ampicillin, cefotaxime, ceftriaxone, ceftazidime, chloramphenicol, ciprofloxacin, co-trimoxazole, ertapenem, imipenem, fluoroquinolones (e.g., ciprofloxacin, gatifloxacin, ofloxacin), streptomycin, sulfadiazine, sulfamethoxazole, tetracycline, and a combination thereof. In a specific embodiment, the administration of the drug or combination of drugs is at least a week, 10 days, two week, three weeks, or four weeks after an infection-producing exposure to the isolate or strain of interest.

In addition, humanized SIRPα-IL-15 non-human animals (e.g., mice) and humanized SIRPα-IL-15 non-human animals (e.g., mice) engrafted with human hematopoietic cells, e.g., engrafted Rag2^{-/-} IL2rg^{Y/-} hSIRPα hIL-15 mice, and optionally having other genetic modifications are useful in studying antibody-dependent cellular cytoxicity (ADCC) mediated by NK cells (e.g., human NK cells). Such animals are also useful models for testing the ability of therapeutic drug candidates, e.g., antigen-binding proteins or antibodies, designed to target various cells (e.g., tumors or infected cells) or infectious agents, to activate NK cell pathways involved in killing such cells or infectious agents.

It is widely known that one of the mechanisms underlying monoclonal antibody therapy is its activation of NK cells through binding the NK cell Fc receptor CD16 (Fc gamma receptor IIIA). Attempts have been made to increase affinity of various known monoclonal candidates (e.g., rituximab) for Fcgamma RIIIA in order to improve ADCC (e.g., Bowles et al. Blood 2006; 108:2648-2654; Garff-Tavernier et al. Leukemia 2011; 25:202-209). As demonstrated herein, the humanized SIRPα-IL-15 engrafted non-human animals produce human NK cells that are capable of mediating ADCC; and thus, these animals present a useful in vivo model for studying ADCC mechanisms and screening various therapeutic candidates.

Thus, engrafted humanized SIRPα-IL-15 non-human animals and cells, e.g., human NK cells, isolated therefrom, may be used in screening methods designed to identify agents which improve antibody dependent cellular cytotoxicity (ADCC) activity of an engrafted cell type in the humanized non-human animal or cells, e.g., human NK cells. For example, a suitable method may include administering an agent to an engrafted humanized SIRPα-IL-15 non-human animal and determining the effect of the agent on an antibody dependent cellular cytotoxicity (ADCC) activity of an engrafted cell type in vivo in the humanized non-human animal. In one embodiment, such effect results in improved tumor killing, e.g., of a transplanted tumor, e.g., of a human tumor. In another embodiment, such effect results in improved killing of infected cell, e.g., virally-infected cell or bacterially-infected cell. In yet another embodiment, such effect results in improved killing of a bacteria, a fungus or a parasite. In various embodiments the agent is an antibody or an antigen-binding protein. In some embodiments, the antibody or the antigen-binding protein is designed to target an antigen expressed on a human tumor cell. In some embodiments, the antibody or the antigen-binding protein is designed to target an antigen expressed on a virally-infected cell or a bacterially-infected cell. In some embodiments, the antibody or the antigen-binding protein is designed to target a bacterial, a fungal, or a parasitic antigen. In some embodiments, an in vitro method is provided wherein human cells, e.g., human NK cells, are isolated from an engrafted humanized SIRPα-IL-15 non-human animal and contacted in vitro with an agent such as an antibody or an antigen-binding protein, and a target cell (e.g., tumor cell) to determine the efficacy of the agent in mediating killing of the target cell. The effect of the agent on the cytolytic activity of the human cells, e.g., human NK cells, can then be determined.

Other examples of uses for the subject mice are provided elsewhere herein. Additional applications of the genetically modified and engrafted mice described in this disclosure will be apparent to those skilled in the art upon reading this disclosure.

### Methods of Making the Subject Genetically Modified Non-Human Animals

In some aspects of the invention, methods are provided for making the subject non-human animals of the present disclosure. In practicing the subject methods, a non-human animal is generated which includes a nucleic acid sequence incorporated into the genome of the genetically modified non-human animal, which sequence encodes a human SIRPα protein and is operably linked to a SIRPα gene promoter, e.g., an endogenous non-human SIRPα gene promoter; and a nucleic acid sequence incorporated into the genome of the genetically modified non-human animal, which sequence encodes a human IL-15 protein and is operably linked to an IL-15 gene promoter, e.g., an endogenous non-human IL-15 gene promoter.

The generation of a non-human animal including a nucleic acid sequence that encodes a human SIRPα protein and is operably linked to a SIRPα promoter, and/or a nucleic acid sequence that encodes a human IL-15 protein and is operably linked to an IL-15 gene promoter, may be accomplished using any convenient method for the making genetically modified animals, e.g. as known in the art or as described herein.

For example, a nucleic acid encoding a human SIRPα protein or a human IL-15 protein may be incorporated into a recombinant vector in a form suitable for insertion into the genome of the host cell and expression of the human protein in a non-human host cell. In various embodiments, the recombinant vector includes the one or more regulatory sequences operatively linked to the nucleic acid encoding the human protein in a manner which allows for transcription of the nucleic acid into mRNA and translation of the mRNA into the human protein, as described above. It will be understood that the design of the vector may depend on such factors as the choice of the host cell to be transfected and/or the amount of human protein to be expressed.

Any of various methods may then be used to introduce the human nucleic acid sequence into an animal cell to produce a genetically modified animal that expresses the human gene. Such techniques are well-known in the art and include, but are not limited to, pronuclear microinjection, transformation of embryonic stem cells, homologous recombination and knock-in techniques. Methods for generating genetically modified animals that can be used include, but are not limited to, those described in Sundberg and Ichiki (2006, Genetically Engineered Mice Handbook, CRC Press), Hofker and van Deursen (2002, Genetically modified Mouse Methods and Protocols, Humana Press), Joyner (2000, Gene Targeting: A Practical Approach, Oxford University Press), Turksen (2002, Embryonic stem cells: Methods and Protocols in Methods Mol Biol., Humana Press), Meyer et al. (2010, Proc. Nat. Acad. Sci. USA 107:15022-15026), and Gibson (2004, A Primer of Genome Science 2^{nd} ed. Sunderland, Massachusetts: Sinauer), U.S. Pat. No. 6,586,251, Rathinam et al. (2011, Blood 118:3119-28), Willinger et al., (2011, Proc Natl Acad Sci USA, 108:2390-2395), Rongvaux et al., (2011, Proc Natl Acad Sci USA, 108:2378-83) and Valenzuela et al. (2003, Nat Biot 21:652-659).

For example, the subject genetically modified animals can be created by introducing the nucleic acid encoding the human protein into an oocyte, e.g., by microinjection, and allowing the oocyte to develop in a female foster animal. In preferred embodiments, the nucleic acid is injected into fertilized oocytes. Fertilized oocytes can be collected from superovulated females the day after mating and injected with the expression construct. The injected oocytes are either cultured overnight or transferred directly into oviducts of 0.5-day p.c. pseudopregnant females. Methods for superovulation, harvesting of oocytes, expression construct injection and embryo transfer are known in the art and described in Manipulating the Mouse Embryo (2002, A Laboratory Manual, 3rd edition, Cold Spring Harbor Laboratory Press). Offspring can be evaluated for the presence of the introduced nucleic acid by DNA analysis (e.g., PCR, Southern blot, DNA sequencing, etc.) or by protein analysis (e.g., ELISA, Western blot, etc.).

As another example, the construct including the nucleic acid sequence encoding the human protein may be transfected into stem cells (e.g., ES cells or iPS cells) using well-known methods, such as electroporation, calcium-phosphate precipitation, lipofection, etc. The cells can be evaluated for the presence of the introduced nucleic acid by DNA analysis (e.g., PCR, Southern blot, DNA sequencing, etc.) or by protein analysis (e.g., ELISA, Western blot, etc.). Cells determined to have incorporated the expression construct can then be introduced into preimplantation embryos. For a detailed description of methods known in the art useful for the compositions and methods of the invention, see Nagy et al., (2002, Manipulating the Mouse Embryo: A Laboratory Manual, 3rd edition, Cold Spring Harbor Laboratory Press), Nagy et al. (1990, Development 110:815-821), U.S. Pat. Nos. 7,576,259, 7,659,442, 7,294,754, and Kraus et al. (2010, Genesis 48:394-399).

In a preferred embodiment, a method of generating a genetically modified animal described herein utilizes a targeting construct made using VELOCIGENE® technology, introducing the construct into ES cells, and introducing targeted ES cell clones into a mouse embryo using VELOCIMOUSE® technology, as described in the Examples.

Genetically modified founder animals can be bred to additional animals carrying the genetic modification. For example, humanized SIRPα non-human animals can be bred with humanized IL-15 non-human animals of the same species to produce the hSIRPα-hIL-15 non-human animals described herein. Genetically modified animals carrying a nucleic acid encoding the human protein(s) of the present disclosure can further be bred to knockout animals, e.g., a non-human animal that is deficient for one or more proteins, e.g. does not express one or more of its genes, e.g. a Rag2-deficient animal and/or an Il2rg-deficient animal.

As discussed above, in some embodiments, the subject genetically modified non-human animal is an immunodeficient animal. Genetically modified non-human animals that are immunodeficient and include one or more human proteins, e.g. hSIRPα and/or hIL-15, may be generated using any convenient method for the generation of genetically modified animals, e.g. as known in the art or as described herein. For example, the generation of the genetically modified immunodeficient animal can be achieved by introduction of the nucleic acid encoding the human protein into an oocyte or stem cells including a mutant SCID gene allele that, when homozygous, will result in immunodeficiency as described in greater detail above and in the working examples herein. Mice are then generated with the modified oocyte or ES cells using, e.g., methods described herein and known in the art, and mated to produce the immunodeficient mice including the desired genetic modification. As another example, genetically modified non-human animals can be generated in an immunocompetent background, and crossed to an animal including a mutant gene allele that, when hemizygous or homozygous, will result in immunodeficiency, and the progeny mated to create an immunodeficient animal expressing the at least one human protein of interest.

In some embodiments, the genetically modified non-human animal is treated so as to eliminate endogenous hematopoietic cells that may exist in the genetically modified non-human animal. In one embodiment, the treatment includes irradiating the genetically modified non-human animal. In a specific embodiment, newborn genetically modified mouse pups are irradated sublethally. In a specific embodiment, newborn pups are irradiated 2×200 cGy with a four hour interval.

Various embodiments of the invention provide genetically modified animals that include a human nucleic acid in substantially all of their cells, as well as genetically modified animals that include a human nucleic acid in some, but not all their cells. In some instances, e.g. targeted recombination, one copy of the human nucleic acid will be integrated into the genome of the genetically modified animals. In other instances, e.g. random integration, multiple copies, adjacent or distant to one another, of the human nucleic acid may be integrated into the genome of the genetically modified animals.

Thus, in some embodiments, the subject genetically modified non-human animal may be an immunodeficient animal including a genome that includes a nucleic acid encoding a human polypeptide operably linked to the corresponding non-human animal promoter, wherein the animal expresses the encoded human polypeptide. In other words, the subject genetically modified immunodeficient non-human animal includes a genome that includes a nucleic acid encoding at least one human polypeptide, wherein the nucleic acid is operably linked to the corresponding non-human promoter and a polyadenylation signal, and wherein the animal expresses the encoded human polypeptide.

### Reagents, Devices and Kits

Also provided are reagents, devices and kits thereof for practicing one or more of the above-described methods. The subject reagents, devices and kits thereof may vary greatly.

In some embodiments, the reagents or kits will include one or more agents for use in the methods described herein. For example, the kit may include a humanized SIRPα-IL-15 non-human animal, e.g., mouse, e.g., a Rag2^{-/-}IL2rg^{Y/-} hSIRPα hIL-15 mouse. The kit may include reagents for breeding humanized SIRPα-IL-15 non-human animals, e.g., mice, e.g., primers and, in some instances, reagents for genotyping humanized SIRPα-IL-15 non-human animals, e.g., mice. The kit may include human hematopoietic cells or an enriched population of human hematopoietic progenitor cells for transplantation into the humanized SIRPα-IL-15 non-human animal, e.g., mouse, or reagents for preparing a population of hematopoietic cells or an enriched population of hematopoietic cells from a human for transplantation into a humanized SIRPα-IL-15 non-human animal, e.g., mouse. Other reagents may include reagents for determining the viability and/or function of hematopoietic cells or differentiated immune cells (e.g., T cells and/or NK cells), e.g. in the presence/absence of candidate agent, e.g., one or more antibodies that are specific for markers expressed by different types of hematopoietic cells or differentiated immune cells (e.g., T cells and/or NK cells), or reagents for detecting particular cytokines, chemokine, etc. Other reagents may include culture media, culture supplements, matrix compositions, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a remote site. Any convenient means may be present in the kits.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-167 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A genetically modified non-human animal, comprising:
   a nucleic acid sequence incorporated into the genome of the genetically modified non-human animal, which sequence encodes a human SIRPα protein and is operably linked to a SIRPα gene promoter; and
   a nucleic acid sequence incorporated into the genome of the genetically modified non-human animal, which sequence encodes a human IL-15 protein and is operably linked to an IL-15 gene promoter, wherein the genetically modified non-human animal expresses the human SIRPα protein and the human IL-15 protein.
2. The genetically modified non-human animal according to 1, wherein the SIRPα gene promoter is an endogenous non-human SIRPα gene promoter.
3. The genetically modified non-human animal according to 2, wherein the SIRPα gene promoter is the endogenous non-human SIRPα gene promoter at the non-human animal SIRPα gene locus.
4. The genetically modified non-human animal according to 3, comprising a null mutation in the non-human SIRPα gene at the non-human animal SIRPα gene locus.
5. The genetically modified non-human animal according to 4, wherein the genetically modified non-human animal is a mouse and the null mutation is a deletion of at least mouse SIRPα exons 2-4.
6. The genetically modified non-human animal according to 4, wherein the genetically modified non-human animal is heterozygous for the allele comprising the nucleic acid sequence that encodes the human SIRPα protein.
7. The genetically modified non-human animal according to 4, wherein the genetically modified non-human animal is homozygous for the allele comprising the nucleic acid sequence that encodes the human SIRPα protein.
8. The genetically modified non-human animal according to any one of 1-7, wherein the nucleic acid sequence that encodes the human SIRPα protein comprises human SIRPα genomic coding and non-coding sequence.
9. The genetically modified non-human animal according to any one of 1-8, wherein the human SIRPα protein is a functional fragment of a full length human SIRPα protein.
10. The genetically modified non-human animal according to 9, wherein the functional fragment comprises an extracellular domain of human SIRPα.
11. The genetically modified non-human animal according to 10, wherein the extracellular domain comprises amino acids 28-362 of SEQ ID NO:12.
12. The genetically modified non-human animal according to any one of 1-11, wherein the IL-15 gene promoter is an endogenous non-human IL-15 gene promoter.
13. The genetically modified non-human animal according to 12, wherein the IL-15 gene promoter is the endogenous non-human IL-15 gene promoter at the non-human animal IL-15 gene locus.
14. The genetically modified non-human animal according to 13, comprising a null mutation in the non-human IL-15 gene at the non-human animal IL-15 gene locus.
15. The genetically modified non-human animal according to 14, wherein the genetically modified non-human animal is a mouse and the null mutation is a deletion of at least mouse IL-15 exons 5-8.
16. The genetically modified non-human animal according to 14, wherein the genetically modified non-human animal is heterozygous for the allele comprising the nucleic acid sequence that encodes the human IL-15 protein.
17. The genetically modified non-human animal according to 14, wherein the genetically modified non-human animal is homozygous for the allele comprising the nucleic acid sequence that encodes the human IL-15 protein.
18. The genetically modified non-human animal according to any one of 1-17, wherein the nucleic acid sequence that encodes the human IL-15 protein comprises human IL-15 genomic coding and non-coding sequence.
19. The genetically modified non-human animal according to any one of 1-18, wherein the human IL-15 protein is a functional fragment of a full length human IL-15 protein.
20. The genetically modified non-human animal according to any one of 1-19, wherein the genetically modified non-human animal is immunodeficient.
21. The genetically modified non-human animal according to 20, wherein the genetically modified non-human animal comprises a Rag2 gene knock-out.
22. The genetically modified non-human animal according to 20 or 21, wherein the genetically modified non-human animal comprises an IL2rg gene knock-out.
23. The genetically modified non-human animal according to any one of 1-22, wherein the non-human animal is a mammal.
24. The genetically modified non-human animal according to 23, wherein the mammal is a rodent.
25. The genetically modified non-human animal according to 24, wherein the rodent is a mouse.
26. The genetically modified non-human animal according to any one of 1-25, wherein the genetically modified non-human animal comprises an engraftment of human hematopoietic cells.
27. The genetically modified non-human animal according to 26, wherein the genetically modified non-human animal comprises an infection with a human pathogen.
28. The genetically modified non-human animal according to 27, wherein the human pathogen activates, induces and/or targets T cells and/or natural killer (NK) cells.
29. The genetically modified non-human animal according to 27, wherein the human pathogen is a pathogen that infects human intestine.
30. The genetically modified non-human animal according to 29, wherein the human pathogen is a human rotavirus.
31. The genetically modified non-human animal according to 27, wherein the pathogen infects human lung.
32. The genetically modified non-human animal according to 31, wherein the human pathogen is an influenza virus.
33. An animal engraftment model, comprising a genetically modified non-human animal comprising:
   a nucleic acid sequence incorporated into the genome of the genetically modified non-human animal, which sequence encodes a human SIRPα protein and is operably linked to a SIRPα gene promoter;
   a nucleic acid sequence incorporated into the genome of the genetically modified non-human animal, which sequence encodes a human IL-15 protein and is operably linked to an IL-15 gene promoter; and
   an engraftment of human hematopoietic cells, wherein the genetically modified non-human animal (i) expresses the human SIRPα protein and the human IL-15 protein, and (ii) comprises human intraepithelial lymphocytes (IELs) in the small intestine and Peyer's patches of the genetically modified non-human animal.
34. The model according to 33, wherein the genetically modified non-human animal comprises an infection with a human pathogen.
35. The model according to 34, wherein the human pathogen is an intestinal pathogen.
36. The model according to 35, wherein the intestinal pathogen is selected from: Campylobacter jejuni, Clostridium difficile, Enterococcus faecalis, Enterococcus faecium, Escherichia coli, Human Rotavirus, Listeria monocytogenes, Norwalk Virus, Salmonella enterica, Shigella flexneri, Shigella sonnei, Shigella dysenteriae, Yersinia pestis, Yersinia enterocolitica, and Helicobacter pylori.
37. The model according to any one of 33-36, wherein the SIRPα gene promoter is an endogenous non-human SIRPα gene promoter.
38. The model according to 37, wherein the SIRPα gene promoter is the endogenous non-human SIRPα gene promoter at the non-human animal SIRPα gene locus.
39. The model according to 38, wherein the genetically modified non-human animal comprises a null mutation in the non-human SIRPα gene at the non-human animal SIRPα gene locus.
40. The model according to 39, wherein the genetically modified non-human animal is a mouse and the null mutation is a deletion of at least mouse SIRPα exons 2-4.
41. The model according to 39, wherein the genetically modified non-human animal is heterozygous for the allele comprising the nucleic acid sequence that encodes the human SIRPα protein.
42. The model according to 39, wherein the genetically modified non-human animal is homozygous for the allele comprising the nucleic acid sequence that encodes the human SIRPα protein.
43. The model according to any one of 33-42, wherein the nucleic acid sequence that encodes the human SIRPα protein comprises human SIRPα genomic coding and non-coding sequence.
44. The model according to any one of 33-43, wherein the human SIRPα protein is a functional fragment of a full length human SIRPα protein.
45. The model according to 44, wherein the functional fragment comprises an extracellular domain of human SIRPα.
46. The model according to 45, wherein the extracellular domain comprises amino acids 28-362 of SEQ ID NO:12.
47. The model according to any one of 33-46, wherein the IL-15 gene promoter is an endogenous non-human IL-15 gene promoter.
48. The model according to 47, wherein the IL-15 gene promoter is the endogenous non-human IL-15 gene promoter at the non-human animal IL-15 gene locus.
49. The model according to 48, wherein the genetically modified non-human animal comprises a null mutation in the non-human IL-15 gene at the non-human animal IL-15 gene locus.
50. The model according to 49, wherein the genetically modified non-human animal is a mouse and the null mutation is a deletion of at least mouse IL-15 exons 5-8.
51. The model according to 48, wherein the genetically modified non-human animal is heterozygous for the allele comprising the nucleic acid sequence that encodes the human IL-15 protein.
52. The model according to 48, wherein the genetically modified non-human animal is homozygous for the allele comprising the nucleic acid sequence that encodes the human IL-15 protein.
53. The model according to any one of 33-52, wherein the nucleic acid sequence that encodes the human IL-15 protein comprises human IL-15 genomic coding and non-coding sequence.
54. The model according to any one of 33-53, wherein the human IL-15 protein is a functional fragment of a full length human IL-15 protein.
55. The model according to any one of 33-54, wherein the genetically modified non-human animal is immunodeficient.
56. The model according to 55, wherein the genetically modified non-human animal comprises a Rag2 gene knock-out.
57. The model according to 55 or 56, wherein the genetically modified non-human animal comprises an IL2rg gene knock-out.
58. The model according to any one of 33-57, wherein the non-human animal is a mammal.
59. The model according to 58, wherein the mammal is a rodent.
60. The model according to 59, wherein the rodent is a mouse.
61. An animal engraftment model, comprising a genetically modified non-human animal comprising:
   a nucleic acid sequence incorporated into the genome of the genetically modified non-human animal, which sequence encodes a human SIRPα protein and is operably linked to a SIRPα gene promoter;
   a nucleic acid sequence incorporated into the genome of the genetically modified non-human animal, which sequence encodes a human IL-15 protein and is operably linked to an IL-15 gene promoter; and
   an engraftment of human hematopoietic cells, wherein the genetically modified non-human animal (i) expresses the human SIRPα protein and the human IL-15 protein, and (ii) comprises human intraepithelial lymphocytes (IELs) in the lung of the genetically modified non-human animal.
62. The model according to 61, wherein the genetically modified non-human animal comprises an infection with a human pathogen.
63. The model according to 62, wherein the human pathogen is lung pathogen.
64. The model according to 63, wherein the lung pathogen is selected from: Streptococcus pyogenes, Haemophilus influenza, Corynebacterium diphtheria, SARS coronavirus, Bordetella pertussis, Moraxella catarrhalis, Influenza virus (A, B, C), Coronavirus, Adenovirus, Respiratory Syncytial Virus, Parainfluenza virus, Mumps virus, Streptococcus pneumoniae, Staphylococcus aureus, Legionella pneumophila, Klebsiella pneumoniae, Pseudomonas aeruginosa, Mycoplasma pneumonia, Mycobacterium tuberculosis, Chlamydia Pneumoniae, Blastomyces dermatitidis, Cryptococcus neoformans, and Aspergillus fumigatus.
65. The model according to any one of 61-64, wherein the SIRPα gene promoter is an endogenous non-human SIRPα gene promoter.
66. The model according to 65, wherein the SIRPα gene promoter is the endogenous non-human SIRPα gene promoter at the non-human animal SIRPα gene locus.
67. The model according to 66, wherein the genetically modified non-human animal comprises a null mutation in the non-human SIRPα gene at the non-human animal SIRPα gene locus.
68. The model according to 67, wherein the genetically modified non-human animal is a mouse and the null mutation is a deletion of at least mouse SIRPα exons 2-4.
69. The model according to 67, wherein the genetically modified non-human animal is heterozygous for the allele comprising the nucleic acid sequence that encodes the human SIRPα protein.
70. The model according to 67, wherein the genetically modified non-human animal is homozygous for the allele comprising the nucleic acid sequence that encodes the human SIRPα protein.
71. The model according to any one of 61-70, wherein the nucleic acid sequence that encodes the human SIRPα protein comprises human SIRPα genomic coding and non-coding sequence.
72. The model according to any one of 61-71, wherein the human SIRPα protein is a functional fragment of a full length human SIRPα protein.
73. The model according to 72, wherein the functional fragment comprises an extracellular domain of human SIRPα.
74. The model according to 73, wherein the extracellular domain comprises amino acids 28-362 of SEQ ID NO:12.
75. The model according to any one of 61-74, wherein the IL-15 gene promoter is an endogenous non-human IL-15 gene promoter.
76. The model according to 75, wherein the IL-15 gene promoter is the endogenous non-human IL-15 gene promoter at the non-human animal IL-15 gene locus.
77. The model according to 76, wherein the genetically modified non-human animal comprises a null mutation in the non-human IL-15 gene at the non-human animal IL-15 gene locus.
78. The model according to 77, wherein the genetically modified non-human animal is a mouse and the null mutation is a deletion of at least mouse IL-15 exons 5-8.
79. The model according to 77, wherein the genetically modified non-human animal is heterozygous for the allele comprising the nucleic acid sequence that encodes the human IL-15 protein.
80. The model according to 77, wherein the genetically modified non-human animal is homozygous for the allele comprising the nucleic acid sequence that encodes the human IL-15 protein.
81. The model according to any one of 61-80, wherein the nucleic acid sequence that encodes the human IL-15 protein comprises human IL-15 genomic coding and non-coding sequence.
82. The model according to any one of 61-80, wherein the human IL-15 protein is a functional fragment of a full length human IL-15 protein.
83. The model according to any one of 61-82, wherein the genetically modified non-human animal is immunodeficient.
84. The model according to 83, wherein the genetically modified non-human animal comprises a Rag2 gene knock-out.
85. The model according to 83 or 84, wherein the genetically modified non-human animal comprises an IL2rg gene knock-out.
86. The model according to any one of 61-85, wherein the non-human animal is a mammal.
87. The model according to 86, wherein the mammal is a rodent.
88. The model according to 87, wherein the rodent is a mouse.
89. A method of determining the efficacy of a candidate T-cell inducing vaccine, the method comprising:
   administering a candidate T-cell inducing vaccine to a genetically modified non-human animal, wherein the genetically modified non-human animal is deficient for an endogenous immune system and comprises:
      (i) a nucleic acid sequence incorporated into the genome of the genetically modified non-human animal, which sequence encodes a human SIRPα protein and is operably linked to a SIRPα gene promoter,
      (ii) a nucleic acid sequence incorporated into the genome of the genetically modified non-human animal, which sequence encodes a human IL-15 protein and is operably linked to an IL-15 gene promoter, and
      (iii) an engraftment of human hematopoietic cells, wherein the genetically modified non-human animal expresses the human SIRPα protein and the human IL-15 protein;
   challenging the genetically modified non-human animal with a human pathogen; and
   determining whether the candidate T-cell inducing vaccine induces a T cell mediated immune response in the genetically modified non-human animal.
90. The method according to 89, wherein the SIRPα gene promoter is an endogenous non-human SIRPα gene promoter.
91. The method according to 90, wherein the SIRPα gene promoter is the endogenous non-human SIRPα gene promoter at the non-human animal SIRPα gene locus.
92. The method according to 91, wherein the genetically modified non-human animal comprises a null mutation in the non-human SIRPα gene at the non-human animal SIRPα gene locus.
93. The method according to 92, wherein the genetically modified non-human animal is a mouse and the null mutation is a deletion of at least mouse SIRPα exons 2-4.
94. The method according to 92, wherein the genetically modified non-human animal is heterozygous for the allele comprising the nucleic acid sequence that encodes the human SIRPα protein.
95. The method according to 92, wherein the genetically modified non-human animal is homozygous for the allele comprising the nucleic acid sequence that encodes the human SIRPα protein.
96. The method according to any one of 89-95, wherein the nucleic acid sequence that encodes the human SIRPα protein comprises human SIRPα genomic coding and non-coding sequence.
97. The method according to any one of 89-96, wherein the human SIRPα protein is a functional fragment of a full length human SIRPα protein.
98. The method according to 97, wherein the functional fragment comprises an extracellular domain of human SIRPα.
99. The method according to 98, wherein the extracellular domain comprises amino acids 28-362 of SEQ ID NO:12.
100. The method according to any one of 89-99, wherein the IL-15 gene promoter is an endogenous non-human IL-15 gene promoter.
101. The method according to 100, wherein the IL-15 gene promoter is the endogenous non-human IL-15 gene promoter at the non-human animal IL-15 gene locus.
102. The method according to 101, wherein the genetically modified non-human animal comprises a null mutation in the non-human IL-15 gene at the non-human animal IL-15 gene locus.
103. The method according to 102, wherein the genetically modified non-human animal is a mouse and the null mutation is a deletion of at least mouse IL-15 exons 5-8.
104. The method according to 101, wherein the genetically modified non-human animal is heterozygous for the allele comprising the nucleic acid sequence that encodes the human IL-15 protein.
105. The method according to 101, wherein the genetically modified non-human animal is homozygous for the allele comprising the nucleic acid sequence that encodes the human IL-15 protein.
106. The method according to any one of 89-105, wherein the nucleic acid sequence that encodes the human IL-15 protein comprises human IL-15 genomic coding and non-coding sequence.
107. The method according to any one of 89-106, wherein the human IL-15 protein is a functional fragment of a full length human IL-15 protein.
108. The method according to any one of 89-107, wherein the genetically modified non-human animal comprises a Rag2 gene knock-out.
109. The method according to any one of 89-108, wherein the genetically modified non-human animal comprises an IL2rg gene knock-out.
110. The method according to any one of 89-109, wherein the genetically modified non-human animal is a mammal.
111. The method according to 110, wherein the mammal is a rodent.
112. The method according to 111, wherein the rodent is a mouse.
113. A method of identifying an agent that inhibits an infection by a pathogen that activates, induces and/or targets human T cells and/or natural killer (NK) cells, the method comprising:
   administering an agent to an genetically modified non-human animal, wherein the genetically modified non-human animal is deficient for an endogenous immune system and comprises:
      (i) a nucleic acid sequence incorporated into the genome of the genetically modified non-human animal, which sequence encodes a human SIRPα protein and is operably linked to a SIRPα gene promoter,
      (ii) a nucleic acid sequence incorporated into the genome of the genetically modified non-human animal, which sequence encodes a human IL-15 protein and is operably linked to an IL-15 gene promoter,
      (iii) an engraftment of human hematopoietic cells, and
      (iv) an infection by a pathogen that activates, induces and/or targets human T cells and/or natural killer cells, wherein the genetically modified non-human animal expresses the human SIRPα protein and the human IL-15 protein; and
   determining whether the agent reduces the amount of the pathogen in the pathogen-infected non-human animal.
114. The method according to 113, wherein the SIRPα gene promoter is an endogenous non-human SIRPα gene promoter.
115. The method according to 114, wherein the SIRPα gene promoter is the endogenous non-human SIRPα gene promoter at the non-human animal SIRPα gene locus.
116. The method according to 115, wherein the genetically modified non-human animal comprises a null mutation in the non-human SIRPα gene at the non-human animal SIRPα gene locus.
117. The method according to 116, wherein the genetically modified non-human animal is a mouse and the null mutation is a deletion of at least mouse SIRPα exons 2-4.
118. The method according to 116, wherein the genetically modified non-human animal is heterozygous for the allele comprising the nucleic acid sequence that encodes the human SIRPα protein.
119. The method according to 116, wherein the genetically modified non-human animal is homozygous for the allele comprising the nucleic acid sequence that encodes the human SIRPα protein.
120. The method according to any one of 113-119, wherein the nucleic acid sequence that encodes the human SIRPα protein comprises human SIRPα genomic coding and non-coding sequence.
121. The method according to any one of 113-120, wherein the human SIRPα protein is a functional fragment of a full length human SIRPα protein.
122. The method according to 121, wherein the functional fragment comprises an extracellular domain of human SIRPα.
123. The method according to 122, wherein the extracellular domain comprises amino acids 28-362 of SEQ ID NO:12.
124. The method according to any one of 113-123, wherein the IL-15 gene promoter is an endogenous non-human IL-15 gene promoter.
125. The method according to 124, wherein the IL-15 gene promoter is the endogenous non-human IL-15 gene promoter at the non-human animal IL-15 gene locus.
126. The method according to 125, the genetically modified non-human animal comprises a null mutation in the non-human IL-15 gene at the non-human animal IL-15 gene locus.
127. The method according to 126, wherein the genetically modified non-human animal is a mouse and the null mutation is a deletion of at least mouse IL-15 exons 5-8.
128. The method according to 125, wherein the genetically modified non-human animal is heterozygous for the allele comprising the nucleic acid sequence that encodes the human IL-15 protein.
129. The method according to 125, wherein the genetically modified non-human animal is homozygous for the allele comprising the nucleic acid sequence that encodes the human IL-15 protein.
130. The method according to any one of 113-129, wherein the nucleic acid sequence that encodes the human IL-15 protein comprises human IL-15 genomic coding and non-coding sequence.
131. The method according to any one of 113-130, wherein the human IL-15 protein is a functional fragment of a full length human IL-15 protein.
132. The method according to any one of 113-131, wherein the genetically modified non-human animal comprises a Rag2 gene knock-out.
133. The method according to any one of 113-132, wherein the genetically modified non-human animal comprises an IL2rg gene knock-out.
134. The method according to any one of 113-133, wherein the genetically modified non-human animal is a mammal.
135. The method according to 134, wherein the mammal is a rodent.
136. The method according to 135, wherein the rodent is a mouse.
137. A method of making a non-human animal expressing a human IL-15 protein and a human SIRPα protein, comprising:
   introducing into a genome of a first non-human animal a nucleic acid sequence encoding a human SIRPα protein, wherein the sequence encoding the human SIRPα protein is operably linked to an SIRPα gene promoter sequence;
   introducing into a genome of a second non-human animal a nucleic acid sequence encoding a human IL-15 protein, wherein the sequence encoding the human IL-15 protein is operably linked to a IL-15 promoter sequence; and
   making a third non-human animal that comprises the nucleic acid sequence encoding the human IL-15 protein and the nucleic acid sequence encoding the human SIRPα protein, wherein the third non-human animal expresses the human IL-15 protein and the human SIPRα protein.
138. The method of 137, wherein the steps of introducing comprise generating a non-human animal from a pluripotent stem cell comprising the nucleic acid encoding human IL-15 or human SIRPα.
139. The method of 137 or 138, wherein the first animal is a different animal than the second animal, and the step of making the third animal comprises breeding the first and the second animal.
140. The method of 137, wherein the first animal and the second animal are the same, the step of introducing into the genome of the first animal comprises contacting a first pluripotent stem cell with the nucleic acid sequence encoding the human SIRPα protein to obtain a second pluripotent stem cell, the step of introducing into the genome of the second animal comprises contacting the second pluripotent stem cell with the nucleic acid sequence encoding the human SIRPα protein to obtain a third pluripotent step cell, and the third non-human animal is made from the third pluripotent stem cell.
141. The method according to any one of 137-140, wherein the pluripotent stem cell is an ES cell or an iPS cell.
142. The method according to any one of 137-140, wherein the pluripotent stem cell is deficient for Rag2.
143. The method according to any one of 137-142, wherein the pluripotent stem cell is deficient for IL2rg.
144. The method according to any one of 137-143, wherein the third non-human animal is deficient in one or both of Rag2 and IL2rg.
145. The method according to any one of 137-144, wherein the IL-15 promoter sequence is a human IL-15 promoter sequence.
146. The method according to any one of 137-144, wherein the IL-15 promoter sequence is an endogenous non-human animal IL-15 promoter sequence.
147. The method according to any one of 137-144, wherein the integration results in a replacement of the non-human IL-15 gene at the non-human IL-15 gene locus.
148. The method according to any one of 137-147, wherein the nucleic acid sequence that encodes the human IL-15 protein comprises human IL-15 genomic coding and non-coding sequence.
149. A method of engrafting a genetically modified non-human animal expressing a human IL-15 protein, comprising:
   transplanting a population of cells comprising human hematopoietic cells into the genetically modified non-human animal made by a method according to any one of 137-148.
150. The method according to 149, wherein the transplanting comprises tail-vein injection, fetal liver injection, or retro-orbital injection.
151. The method according to 149 or 150, wherein the genetically modified non-human animal is sublethally irradiated prior to transplantation.
152. The method according to any one of 149-151, wherein the human hematopoietic cells are CD34+ cells.
153. The method according to any one of 149-151, wherein the human hematopoietic cells are from fetal liver, adult bone marrow, or umbilical cord blood.
154. A method of determining the efficacy of a candidate therapeutic antibody or antigen-binding protein in killing a target cell, the method comprising:
   administering the candidate therapeutic antibody or antigen-binding protein to a genetically modified non-human animal, wherein the genetically modified non-human animal is deficient for an endogenous immune system and comprises:
      (i) a nucleic acid sequence incorporated into the genome of the genetically modified non-human animal, which sequence encodes a human SIRPα protein and is operably linked to a SIRPα gene promoter,
      (ii) a nucleic acid sequence incorporated into the genome of the genetically modified non-human animal, which sequence encodes a human IL-15 protein and is operably linked to an IL-15 gene promoter, and
      (iii) an engraftment of human hematopoietic cells, wherein the genetically modified non-human animal expresses the human SIRPα protein and the human IL-15 protein; and
   determining whether the candidate therapeutic antibody or antigen-binding protein modulates an NK cell mediated antibody-dependent cellular cytotoxicity against the target cell in the genetically modified non-human animal.
155. A method of determining the efficacy of a candidate therapeutic antibody or antigen-binding protein in killing a target cell, the method comprising:
   isolating an NK cell from a genetically modified non-human animal, wherein the genetically modified non-human animal is deficient for an endogenous immune system and comprises:
      (i) a nucleic acid sequence incorporated into the genome of the genetically modified non-human animal, which sequence encodes a human SIRPα protein and is operably linked to a SIRPα gene promoter,
   (ii) a nucleic acid sequence incorporated into the genome of the genetically modified non-human animal, which sequence encodes a human IL-15 protein and is operably linked to an IL-15 gene promoter, and
   (iii) an engraftment of human hematopoietic cells, wherein the genetically modified non-human animal expresses the human SIRPα protein and the human IL-15 protein;
   contacting the isolated NK cell with the candidate therapeutic antibody or antigen-binding protein and the target cell; and
   determining the antibody- or the antigen-binding protein-dependent cytolytic activity of the isolated NK cell against the target cell.
156. A method of screening a candidate therapeutic antibody or antigen-binding protein for improved efficacy in killing a target cell comprising:
   administering the candidate therapeutic antibody or antigen-binding protein to a genetically modified non-human animal, wherein the genetically modified non-human animal is deficient for an endogenous immune system and comprises:
      (i) a nucleic acid sequence incorporated into the genome of the genetically modified non-human animal, which sequence encodes a human SIRPα protein and is operably linked to a SIRPα gene promoter,
      (ii) a nucleic acid sequence incorporated into the genome of the genetically modified non-human animal, which sequence encodes a human IL-15 protein and is operably linked to an IL-15 gene promoter, and
      (iii) an engraftment of human hematopoietic cells, wherein the genetically modified non-human animal expresses the human SIRPα protein and the human IL-15 protein; and
   determining whether the candidate therapeutic antibody or antigen-binding protein displays improved efficacy in killing the target cell in the genetically modified non-human animal.
157. The method of any one of 154-156, wherein the target cell is selected from the group consisting of a tumor cell, a virally-infected cell, a bacterially-infected cell, a bacterial cell, a fungal cell, and a parasitic cell.
158. A method of determining the efficacy a candidate therapeutic antibody or antigen-binding protein in NK-cell mediated killing of a target cell, comprising:
   administering the candidate therapeutic antibody or antigen-binding protein to a genetically modified non-human animal, wherein the genetically modified non-human animal is deficient for an endogenous immune system and comprises:
      (i) a nucleic acid sequence incorporated into the genome of the genetically modified non-human animal, which sequence encodes a human SIRPα protein and is operably linked to a SIRPα gene promoter,
      (ii) a nucleic acid sequence incorporated into the genome of the genetically modified non-human animal, which sequence encodes a human IL-15 protein and is operably linked to an IL-15 gene promoter, and
      (iii) an engraftment of human hematopoietic cells, wherein the genetically modified non-human animal expresses the human SIRPα protein and the human IL-15 protein; and
   determining whether the candidate therapeutic antibody or antigen-binding protein modulates (e.g., activates) NK cell antibody-dependent cellular cytotoxicity against the target cell in the genetically modified non-human animal.
159. The method of 158, wherein the target cell is selected from the group consisting of a tumor cell, a virally-infected cell, a bacterially-infected cell, a bacterial cell, a fungal cell, and a parasitic cell.
160. The method of claim 159, wherein the target cell is a tumor cell.
161. The method of claim 160, wherein the tumor cell is a B-cell lymphoma cell.
162. A model of NK cell mediated antibody-dependent cellular cytotoxicity, comprising a genetically modified non-human animal, wherein the genetically
modified non-human animal is deficient for an endogenous immune system and comprises:
   a nucleic acid sequence incorporated into the genome of the genetically modified non-human animal, which sequence encodes a human SIRPα protein and is operably linked to a SIRPα gene promoter;
   a nucleic acid sequence incorporated into the genome of the genetically modified non-human animal, which sequence encodes a human IL-15 protein and is operably linked to an IL-15 gene promoter; and
   an engraftment of human hematopoietic cells, wherein the genetically modified non-human animal (i) expresses the human SIRPα protein and the human IL-15 protein, (ii) comprises human lymphocytes, and (iii) comprises a target cell selected from the group consisting of a tumor cell, a virally-infected cell, a bacterially-infected cell, a bacterial cell, a fungal cell, and a parasitic cell.
163. The model of claim 162, wherein the target cell is a tumor cell.
164. The model of claim 163, wherein the tumor cell is a B-cell lymphoma cell.
165. The model of claim 163 or claim 164, wherein the model comprises an exogenous candidate therapeutic antibody or antigen-binding protein.
166. The model of any one of claims 162-165, wherein the genetically modified non-human animal comprises human intraepithelial lymphocytes (IELs) in the small intestine and Peyer's patches of the genetically modified non-human animal.
167. The model of any one of claims 162-166, wherein the genetically modified non-human animal comprises human intraepithelial lymphocytes (IELs) in the lung of the genetically modified non-human animal.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Generation of Humanized SIRPα (SRG) Knock-In Mice

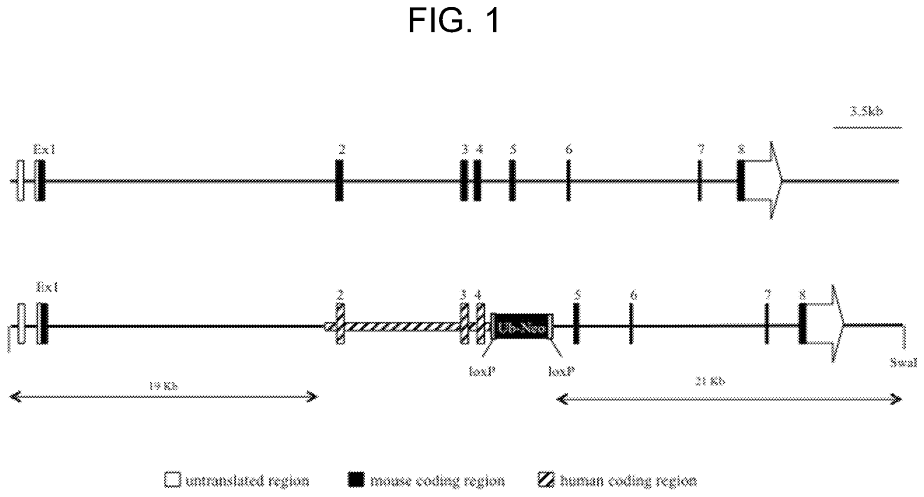

A human SIRPα knock-in mouse was generated, which expresses the extracellular domain of human SIRPα operably linked to the mouse SIRPα promoter (see FIG. 1). Human SIRPα is known to exist in at least 10 allelic forms. In this particular example, human SIRPα variant 1 is employed for humanizing an endogenous SIRPα gene of a mouse.

### Materials and Methods

The generation of knock-in mice encoding human SIRPα into the Rag2^{-/-} Il2rg^{Y/-} 129xBalb/c (N2) genetic background was performed using VELOCIGENE® technology as described in greater detail below. The mice were maintained under specific pathogen-free conditions and with continuous treatment of enrofloxacin in the drinking water (Baytril; 0.27 mg/mL).

A targeting vector for humanization of an extracellular region of a SIRPα (e.g., SIRPα) gene was constructed using VELOCIGENE® technology (see, e.g., U.S. Pat. No. 6,586,251 and Valenzuela et al. (2003) High-throughput engineering of the mouse genome coupled with high-resolution expression analysis, Nature Biotech. 21 (6): 652-659).

Briefly, mouse bacterial artificial chromosome (BAC) clone bMQ-261H14 was modified to delete the sequence containing exons 2 to 4 of an endogenous SIRPα gene and insert exons 2 to 4 of a human SIRPα gene using human BAC clone CTD-3035H21. The genomic DNA corresponding to exons 2 to 4 of an endogenous SIRPα gene (~8555 bp) was replaced in BAC clone bMQ-261H14 with a ~8581 bp DNA fragment containing exons 2 to 4 of a human SIRPα gene from BAC clone CTD-3035H21. Sequence analysis of the human SIRPα allele contained in BAC clone CTD-3035H21 revealed the allele to correspond to human variant 1. A neomycin cassette flanked by loxP sites was added to the end of the ~8581 bp human DNA fragment containing exons 2 to 4 of the human SIRPα gene (FIG. 1 (bottom)).

Upstream and downstream homology arms were obtained from mouse BAC DNA at positions 5′ and 3′ of exons 2 and 4, respectively, and added to the ~8581 bp human fragment-neomycin cassette to create the final targeting vector for humanization of an endogenous SIRPα gene, which contained from 5′ to 3′ a 5′ homology arm containing 19 kb of mouse DNA 5′ of exon 2 of the endogenous SIRPα gene, a ~8581 bp DNA fragment containing exons 2 to 4 of a human SIRPα gene, a neomycin cassette flanked by loxP sites, and a 3′ homology arm containing 21 kb of mouse DNA 3′ of exon 4 of an endogenous SIRPα gene. Targeted insertion of the targeting vector positioned the neomycin cassette in the fifth intron of a mouse SIRPα gene between exons 4 and 5. The targeting vector was linearized by digesting with SwaI and then used in homologous recombination in bacterial cells to achieve a targeted replacement of exons 2 to 4 in a mouse SIRPα gene with exons 2 to 4 of a human SIRPα gene (FIG. 1 (bottom)).

The targeted BAC DNA (described above) was used to electroporate Rag2^{-/-} IL2rg^{Y/-} mouse ES cells to create modified ES cells including a replacement of exons 2 to 4 in an endogenous mouse SIRPα gene with a genomic fragment including exons 2 to 4 of a human SIRPα gene. Positive ES cells containing a genomic fragment including exons 2 to 4 of a human SIRPα gene were identified by quantitative PCR using TAQMAN™ probes (Lie and Petropoulos, 1998. Curr. Opin. Biotechnology 9:43-48). The nucleotide sequence across the upstream insertion point included the following, which indicates endogenous mouse sequence upstream of the insertion point (contained within the parentheses below) linked contiguously to a human SIRPα genomic sequence present at the insertion point:

(SEQ ID NO: 1)
(AGCTCTCCTACCACTAGACTGCTGAGACCCGCTGCTCTGCTCAGGACT
CGATTTCCAGTACACAATCTCCCTCTTTGAAAAGTACCACACATCCTGG
GGT)GCTCTTGCATTTGTGTGACACTTTGCTAGCCAGGCTCAGTCCTGG
GTTCCAGGTGGGGACTCAAACACACTGGCACGAGTCTACATTGGATATT
CTTGGT.

The nucleotide sequence across the downstream insertion point at the 5′ end of the neomycin cassette included the following, which indicates human SIRPα genomic sequence contiguous with cassette sequence downstream of the insertion point (contained within the parentheses below with loxP sequence italicized):

(SEQ ID NO: 2)
GCTCCCCATTCCTCACTGGCCCAGCCCCTCTTCCCTACTCTTTCTAGCC
CCTGCCTCATCTCCCTGGCTGCCATTGGGAGCCTGCCCCACTGGAAGCC
AG(TCGAGATAACTTCGTATAATGTATGCTATACGAAGTTATATGCATG
GCCTCCGCGCCGGGTTTTGGCGCCTCCCGCGGGCGCCCCCCTCCTCACG
GCGA).

The nucleotide sequence across the downstream insertion point at the 3′ end of the neomycin cassette included the following, which indicates cassette sequence contiguous with mouse genomic sequence 3′ of exon 4 of an endogenous SIRPα gene (contained within the parentheses below):

(SEQ ID NO: 3)
CATTCTCAGTATTGTTTTGCCAAGTTCTAATTCCATCAGACCTCGACC
TGCAGCCCCTAGATAACTTCGTATAATGTATGCTATACGAAGTTATGC
TAGC(TGTCTCATAGAGGCTGGCGATCTGGCTCAGGGACAGCCAGTAC
TGCAAAGAGTATCCTTGTTCATACCTTCTCCTAGTGGCCATCTCCCTG
GGACAGTCA).

Positive ES cell clones were then used to implant female mice using the VELOCIMOUSE® method (see, e.g., U.S. Pat. No. 7,294,754 and Poueymirou et al. 2007, F0 generation mice that are essentially fully derived from the donor gene-targeted ES cells allowing immediate phenotypic analyses, Nature Biotech. 25(1):91-99) to generate a litter of pups containing an insertion of exons 2 to 4 of a human SIRPα gene into an endogenous SIRPα gene of a mouse.

Targeted ES cells described above were used as donor ES cells and introduced into an 8-cell stage mouse embryo by the VELOCIMOUSE® method (supra). Mice bearing the humanization of exons 2 to 4 of an endogenous SIRPα gene were identified by genotyping using a modification of allele assay (Valenzuela et al., supra) that detected the presence of the human SIRPα gene sequences.

Mice bearing the humanized SIRPα gene construct (i.e., containing human SIRPα exons 2 to 4 in a mouse SIRPα gene) can be bred to a Cre deletor mouse strain (see, e.g., International Patent Application Publication No. WO 2009/114400) in order to remove any loxed neomycin cassette introduced by the targeting vector that is not removed, e.g., at the ES cell stage or in the embryo. Optionally, the neomycin cassette is retained in the mice. To obtain homozygous Sirpa mice heterozygotes are bred.

### Results

Mice including a nucleic acid encoding a humanized version of the mouse SIRPα gene as described above (SRG mice) exhibit physiological expression of a humanized SIRPα protein (data not shown). These mice also exhibit human immune cell engraftment in the spleen, peripheral lymph nodes (LN) and thymus comparable to NOD scid gamma (NSG) mice (data not shown).

### Example 2: Generation of Humanized SRG IL-15 h/h (SRG-15) Knock-In Mice

The cytokine IL-15 has been shown to be important for mouse NK cell development and memory CD8⁺ T cell differentiation and maintenance. To study the effects of human IL-15 on the development, differentiation and maintenance of human immune cells in the context of an animal model, human IL-15 human SIRPα knock-in mice were generated as described in greater detail below. FIG. 2 shows a schematic representation of the IL-15 knock-in construct.

### Materials and Methods

Mouse ES cells were modified to replace mouse IL-15 gene sequence with human IL-15 gene sequence at the endogenous mouse IL-15 locus, under control of mouse IL-15 regulatory elements, using VELOCIGENE® genetic engineering technology, to produce a humanized locus as shown in FIG. 2. Knock-in mice comprising human Il-15 were generated on Rag2^{-/-} Il2rg^{Y/-} 129xBalb/c genetic background. FIG. 2 does not show upstream (with respect to direction of transcription of the IL-15 gene) the 5′ untranslated exons of the mouse gene (exons 1 and 2); coding exon 1 (exon 3) of FIG. 2 shows a small untranslated region (unfilled) upstream of the coding exon. Except as discussed below for mouse 1, as shown in the humanization at the bottom of FIG. 2, mouse coding exons 1 and 2 (exons 3 and 4) were retained, whereas mouse coding exons 3 through 6 (exons 5-8) were replaced with human coding exons 3 through 6 (exons 5-8). At the downstream end, human coding exon 6 (exon 8) is followed by a stop codon and a human 3′-UTR, and further by human sequence found downstream of the human 3′UTR. For selection purposes, a selection cassette (floxed for removal by Cre) was included. The humanized locus of FIG. 2 expresses a mature IL-15 protein that is fully human.

Specifically, bacterial homologous recombination (BHR) was performed to construct a large targeting vector (LTVEC) containing sequences of the human IL-15 gene for targeting to the mouse IL-15 locus using standard BHR techniques (see, e.g., Valenzuela et al. (2003), supra) and gap repair BHR. Linear fragments were generated by ligating PCR-generated homology boxes to cloned cassettes followed by gel isolation of ligation products and electroporation into BHR-competent bacteria harboring the target bacterial artificial chromosome (BAC). Mouse BAC PRCI23-203P7 is used as the source of mouse sequence; human BAC RP11-103B12 is used as the source of human IL-15 gene sequence. Following a selection step, correctly recombined clones are identified by PCR across novel junctions, and by restriction analysis. An LTVEC containing homology arms and human IL-15 gene sequences was made.

The mouse IL-15 gene (mouse GeneID: 103014; RefSeq transcript: NM_008357.2; ensemble eID:16168) is modified by using genomic coordinates for deletion GRCM38: ch 8:82331173-82343471 (minus strand); genomic coordinates for replacement GRCh37: ch4: 142642924-142655819 (plus strand). 12299 nucleotides of mouse sequence were replaced by 12896 nucleotides of human sequence. The replacement of mouse IL-15 sequence as described above is graphically presented in FIG. 2.

The LTVEC including the humanized IL-15 gene had about 13 kb of upstream mouse targeting arm flanked upstream with a MluI site, and a 27 kb downstream mouse targeting arm flanked downstream with an AscI site. The LTVEC was linearized with MluI and AscI for electroporation.

Following construction of the LTVEC, nucleotide sequence of the LTVEC across the mouse/human 5′ junction, and human/mouse 3′ junction is as shown in Table 1 below. SEQ ID NO:4 depicts the upstream (with respect to direction of transcription of the IL-15 gene) junction between mouse sequence and human sequence; the sequence shown begins with mouse sequence in uppercase, followed by an AsisI restriction site in lowercase, followed by human IL-15 nucleic acid sequence in uppercase. SEQ ID NO:5 indicates downstream human IL-15 coding and noncoding sequence in uppercase (human 3′UTR bolded italics), followed by an XhoI site in lowercase, followed by a lox site (uppercase, bolded italics), followed by sequence of the downstream neo selection cassette (uppercase), which extends 2.6 kb downstream (not shown). SEQ ID NO:6 is a nucleic acid sequence that depicts the junction between the downstream portion of the neo selection cassette (uppercase), with lox site (uppercase and bolded italics), followed by an Nhel site (lowercase), which is followed by mouse sequence downstream of the humanization (uppercase); the selection cassette extends 2.6 kb further upstream.

TABLE 1
Junction Sequences of Humanized IL-15 Locus

| SEQ ID NO | Sequence |
|---|---|
| SEQ ID NO: 4 | ATCCATTTAGCCTTTCTCTGATCACTAAGTTGGACAGTTGGA |
| | CAGTCTTCCTCAAATTAGCTTAGACTATCAAAATTATACTGT |
| | ATTTTTGGTATTTCCAgcgatcgcTTCAGTTACAAGGCTGTTGAA |
| | TGCACAGAAGCAAGGATAACACTGATTTTTTCACTGGTCAG |
| | AATAAAAATTATTGATTGCTCTTTTGCTTATAGTATTC |
| SEQ ID NO: 5 | AATGTAACAGAATCTGGATGCAAAGAATGTGAGGAACTGG |
| | AGGAAAAAAATATTAAAGAATTTTTGCAGAGTTTTGTACAT |
| | ATTGTCCAAATGTTCATCAACACTTCTTGATTGCAATTGATT |
| | CTTTTTAAAGTGTTTCTGTTATTAACAAACATCACTCTGCTG |
| | CTTAGACATAACAAAACACTCGGCATTTCAAATGTGCTGTCA |
| | AAACAAGTTTTTCTGTCAAGAAGATGATCAGACCTTGGATCA |
| | GATGAACTCTTAGAAATGAAGGCAGAAAAATGTCATTGAGTA |
| | ATATAGTGACTATGAACTTCTCTCAGACTTACTTTACTCATTT |
| | TTTTAATTTATTATTGAAATTGTACATATTTGTGGAATAATGT |
| | AAAATGTTGAATAAAAATATGTACAAGTGTTGTTTTTTAAGTT |
| | GCACTGATATTTTACCTCTTATTGCAAAATAGCATTTGTTTAA |
| | GGGTGATAGTCAAATTATGTATTGGTGGGGCTGGGTACCAAT |
| | GCTGCAGGTCAACAGCTATGCTGGTAGGCTCCTGCCAGTGTG |
| | GAACCACTGACTACTGGCTCTCATTGACTTCCTTACTAAGCAT |
| | AGCAAACAGAGGAAGAATTTGTTATCAGTAAGAAAAAGAAGA |
| | ACTATATGTGAATCCTCTTCTTTATACTGTAATTTAGTTATTG |
| | ATGTATAAAGCAACTGTTATGAAATAAAGAAATTGCAATAACT |
| | GGCATATAATGTCCATCAGTAAATCTTGGTGGTGGTGGCAA |
| | TAATAAACTTCTACTGATAGGTAGAATGGTGTGCAAGCTTG |
| | TCCAATCACGGATTGCAGGCCACATGCGGCCCAGGACAACT |
| | TTGAATGTGGCCCAACACAAATTCATAAACTTTCATACATCT |
| | CGTTTTTAGCTCATCAGCTATCATTAGCGGTAGTGTATTTAA |
| | AGTGTGGCCCAAGACAATTCTTCTTATTCCAATGTGGCCCA |
| | GGGAAATCAAAAGATTGGATGCCCCTGGTATAGAAAACTA |
| | ATAGTGACAGTGTTCATATTTCATGCTTTCCCAAATACAGGT |
| | ATTTTATTTTCACATTCTTTTTGCCATGTTTATATAATAATAA |
| | AGAAAAACCCTGTTGATTTGTTGGAGCCATTGTTATCTGAC |
| | AGAAAATAATTGTTTATATTTTTTGCACTACACTGTCTAAAA |
| | TTAGCAAGCTCTCTTCTAATGGAACTGTAAGAAAGATGAAA |
| | TATTTTTGTTTTATTATAAATTTATTTCACCTTAATTCTGGTA |
| | ATACTCACTGAGTGACTGTGGGGTGGGAAATGATCTCTTAA |
| | GAATTTGATTTCTTTCTATTCCATAGTACAAACTCGTTCTCT |
| | GTTGAAACATTCTTCTATCACCCCAGTGCCCTATCCATGTAC |
| | ATGTGTTCTTATTGCTCTAGTCAAACGGTGCTTATAAATATC |
| | TTTCAGAAAGTTTAGGAGAAATCTGTATCCTATTTGACTTCC |
| | AATAATCATGTATTGGCTGTCAGCTTCTTACCTACTCTCAGT |
| | CCAGAGAAATAGTATTTGGCAGCCACTCTTTAAAGTTTATG |
| | GGTTGTGGATTGTGGCGGTTGATTTATTTTTTTTATTTCAATT |
| | GGGATAGAATTTTTTAATATACCTGTATTTTTGTTTTGTTTTA |
| | TGTAGCTTTTCTATTAGGGAGAGTAGGAAAAGTGCACCATT |
| | TTCTTCTCTAAATTTCCAGTCCAGTCTTTAGGGGAATGTTAG |
| | TCTTCCTGAGATGGGGGAAGGAAAATCATAATGCCAGTCAC |
| | TTTGCAAATAATATTTTATAGTGATAAATGGTTCATTTTGGT |
| | TACATAGGCATACAAGTGGGCTTAAAACTTGGAATTTACCA |
| | GGGCTCAAAATTAAAATTCTTACATTAGTTACTCGATATGG |
| | ATCGCTTCAGTTGATCTTAGAAAACTCAAGGCATAGATCTG |
| | CAACctcgagATAACTTCGTATAATGTATGCTATACGAAGTTATA |
| | TGCATGGCCTCCGCGCCGGGTTTTGGCGCCTCCCGCGGGCG |
| | CCCCCCTCCTCACGGCG |
| SEQ ID NO: 6 | CATTCTCAGTATTGTTTTGCCAAGTTCTAATTCCATCAG |
| | ACCTCGACCTGCAGCCCCTAGATAACTTCGTATAATGT |
| | ATGCTATACGAAGTTATgctagcGTGATAGTCCTTCACG |
| | GAAAGTACAAGAATACACAGAAAACTGCTGTTTACATT |
| | AGTCTTTCACGTTTTTATTTTATTCTCACAAATTTTAATGCAATAC |

Mouse ES cells were electroporated with the LTVEC constructs, grown on selection medium, and used as donor ES cells to make humanized IL-15 mice including a replacement at the endogenous mouse IL-15 locus with human sequence as depicted in FIG. 2. Following electroporation of the ES cell, a loss of native allele assay (see, e.g., Valenzuela et al. (2003), supra) is performed to detect loss of endogenous IL-15 sequence due to the targeting.

Correctly targeted ES cells were further electroporated with a transient Cre-expressing vector to remove the Neo drug selection cassette.

Donor mouse ES cells including a humanized IL-15 locus were introduced into early stage mouse embryos by the VELOCIMOUSE® method (Poueymirou et al. (2007) F0 generation mice fully derived from gene-targeted embryonic stem cells allowing immediate phenotypic analyses, Nat Biotechnol 25:91-99). Heterozygous mice were obtained, and heterozygotes were bred to obtain homozygotes with respect to humanized IL-15. Two versions of humanized IL-15 mice were generated (referred to herein as mouse 1 and mouse 2). Following further analysis, the mouse 1 version was found to contain an exon duplication in its genome. In mouse 2 the endogenous mouse IL-15 locus was replaced with human sequence as depicted in FIG. 2.

Human IL-15mRNA levels were determined as follows. Reverse transcription (RT)-qPCR was performed using a 7500 Fast Real-Time PCR System (Applied Biosystems) and a SYBR® FAST universal qPCR kit (KAPA Biosystems). Sequence-specific oligonucleotide primers were designed using Primer3 software and synthesized by Sigma-Aldrich. The following primers were used: mouse Hprt forward: 5′-AGGGATTTGAATCACGTTTG-3′(SEQ ID NO:7), mouse Hprt reverse: 5′-TTTACTGGCAACATCAACAG-3′(SEQ ID NO:8); human Ill5 forward: 5′-GCCCAGGGAAATCAAAAGAT-3′(SEQ ID NO:9), human Il15 reverse: 5′-TGGCTCCAACAAATCAACAG-3′ (SEQ ID NO:10). Relative expression values were calculated using the comparative threshold cycle method and normalized to mouse Hprt.

SRG-15 mice are generated either by (1) breeding mice comprising human SIRPα replacement to mice comprising human IL-15 replacement, both on Rag2^{-/-} Il2rg^{Y/-} background, or by (2) introducing a large targeting vector comprising human IL-15 into an ES cell harboring human SIRPα replacement on Rag2^{-/-} Ilrg^{Y/-} background (described in Example 1) and generating mice from ES cells harboring both human IL-15 and SIRPα gene replacements as well as Rag2^{-/-} Il2rg^{Y/-} using the VELOCIMOUSE® method. Heterozygous mice are bred to homozygosity.

### Results

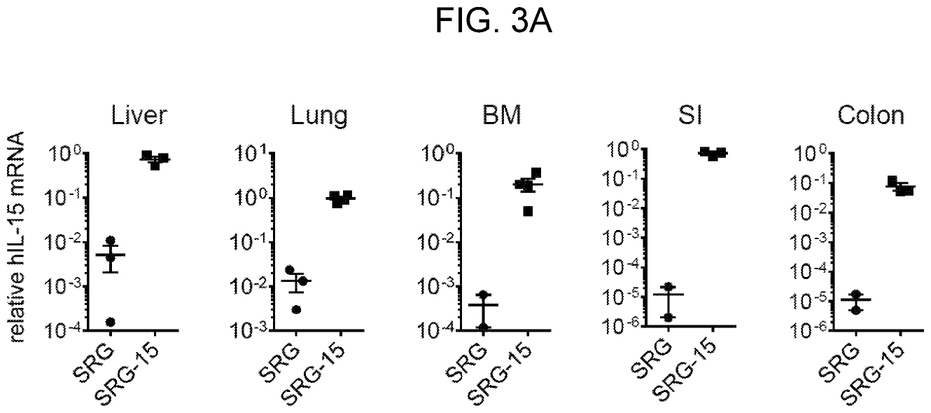

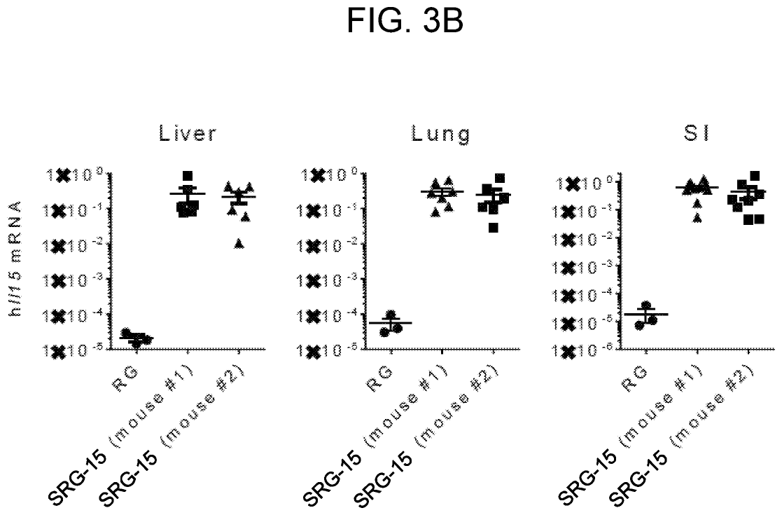

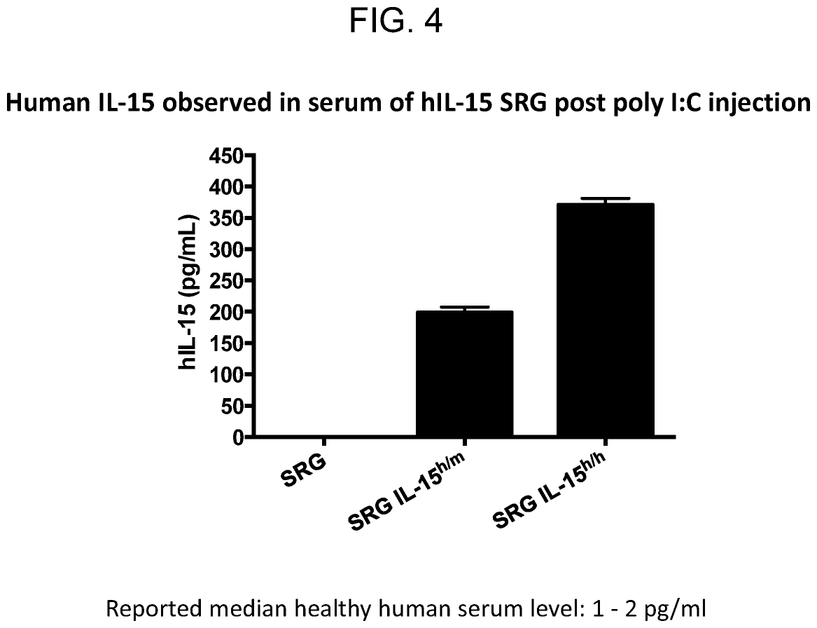

As illustrated in FIGS. 3A and 3B, high levels of expression of human IL-15 mRNA were found in the liver, lung, bone marrow (BM), small intestine (SI) and colon of non-engrafted SRG-15 mouse 1. Similarly high levels of human IL-15 mRNA were found in the liver, lung and small intestine of non-engrafted SRG-15 mouse 2 (FIG. 3B). As shown in FIG. 4, upon stimulation by poly (I:C), high levels of human IL-15 protein could also be detected in the serum of SRG-15 mouse 2, wherein human exons 5-8 replace the endogenous mouse exons.

### Example 3: Engraftment of SRG-15 Mice

### Materials and Methods

SRG and SRG-15 mice are engrafted as described below. Neonate mice are irradiated sub-lethally without anesthesia 3-5 days post birth with 160cGy and returned to their mothers for rest. 4-12 hours post irradiation these neonates are transplanted with CD34+ huHSCs in 25 μl PBS intrahepatically (i.h.) using a 30G needle.

### Results

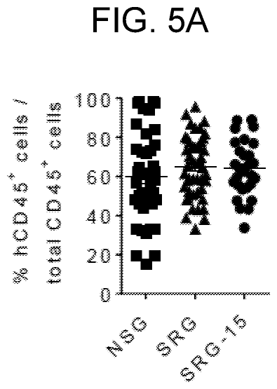

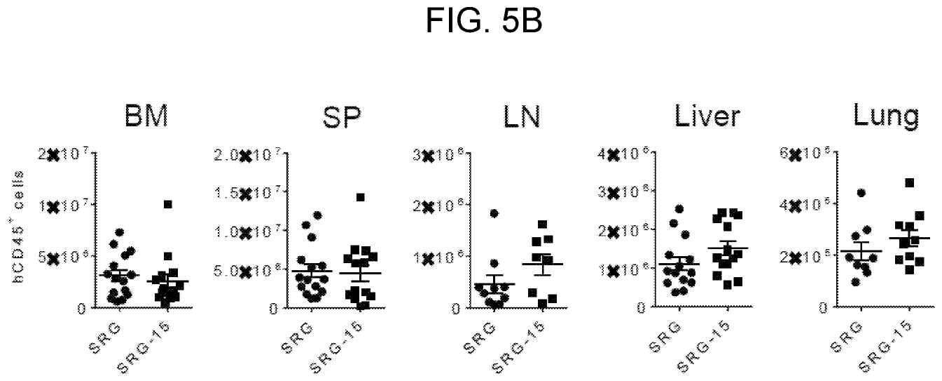

To assess the impact of human IL-15 on immune cell development, human CD45⁺ cell engraftment in NSG, SRG and SRG-15 mice was compared. Efficient engraftment of human hematopoietic cells in the blood of NSG, SRG and SRG-15 (mouse 2) mice was seen 12-14 weeks post engraftment as shown in FIG. 5A. A comparison showing engraftment as evidenced by human CD45+ cell numbers in the BM, spleen, LN, liver and lung of SRG and SRG-15 (mouse 2) 14 weeks post engraftment is provided in FIG. 5B.

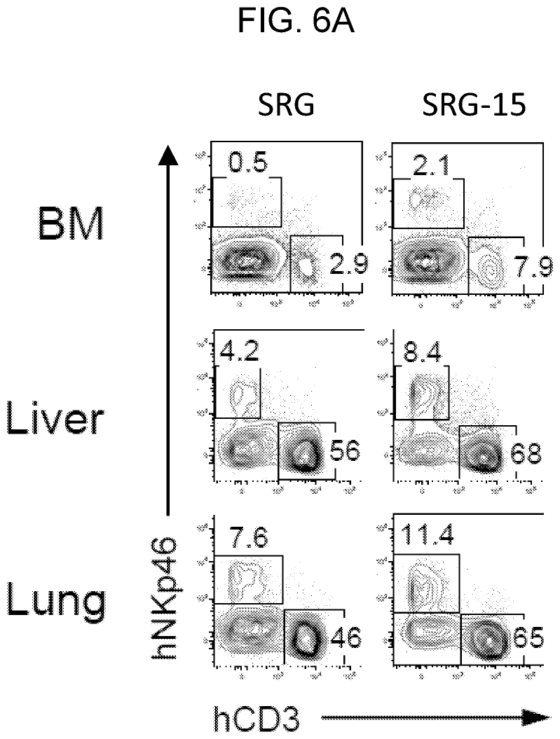

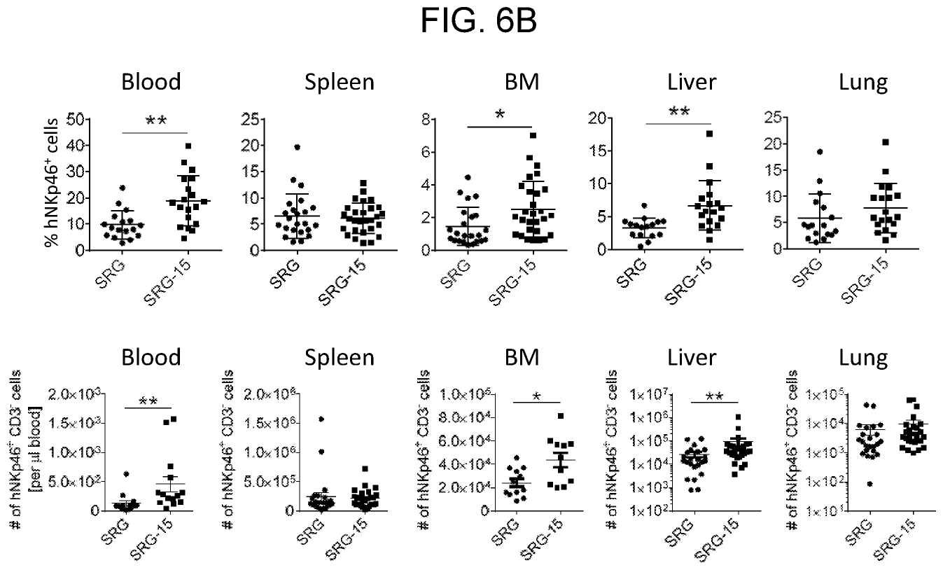

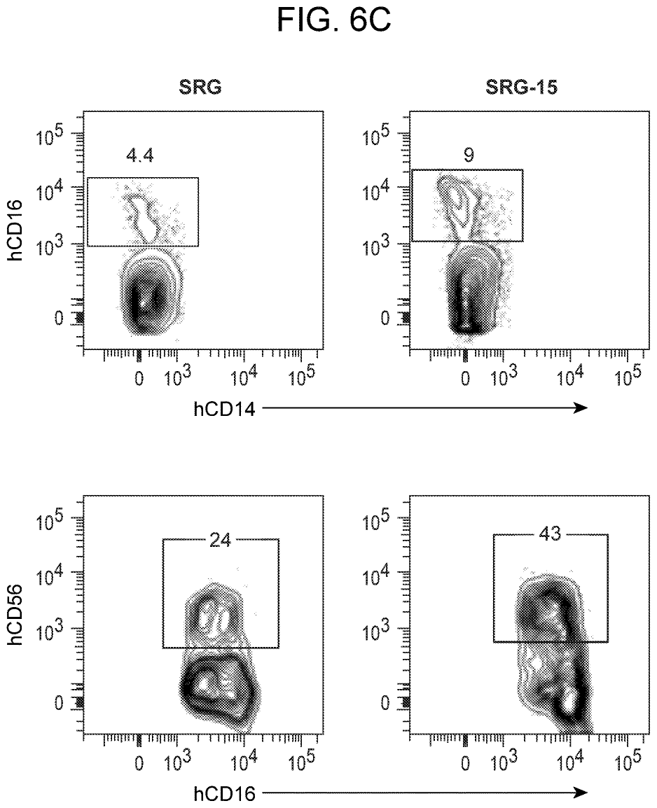

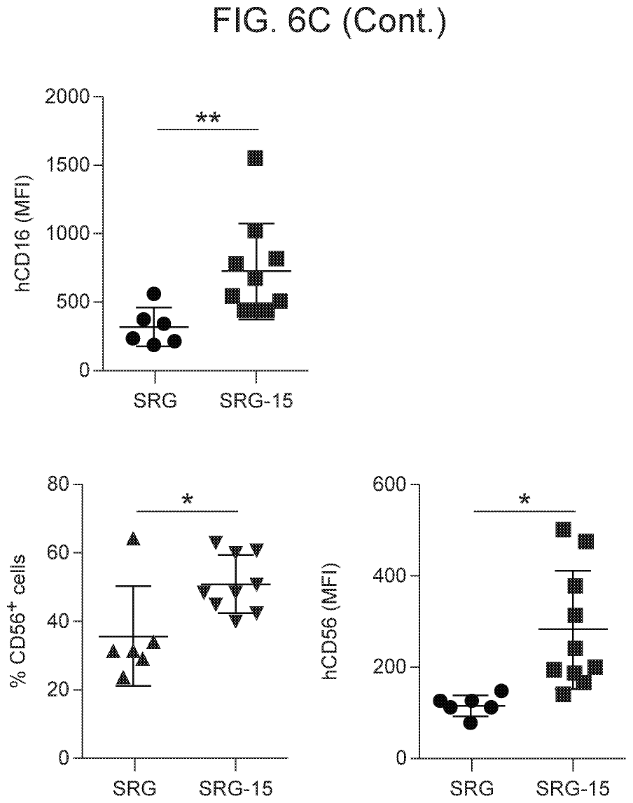

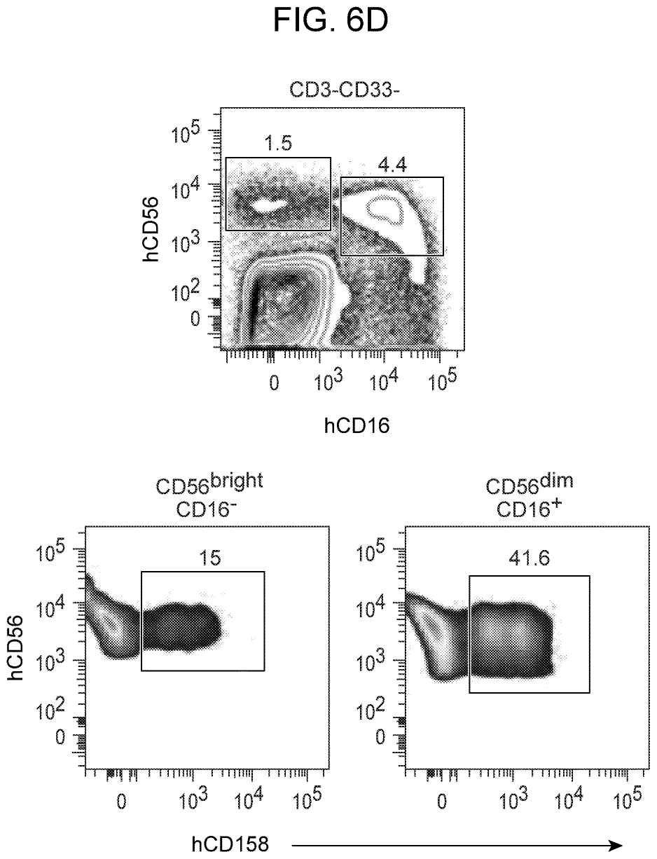

In mouse 1, although human CD45⁺ cell engraftment was not different, a higher percentage and number of human NK cells was found in various tissues in SRG-15 mice compared to SRG mice, as illustrated by FIGS. 6A and 6B. IL-15 is not only important for NK cell development and survival but also for their maturation. As shown in FIG. 6C, human NK cells in the liver of SRG-15 mice (mouse 1) had a higher expression level of CD16 and CD56, indicating increased NK cell maturation in SRG-15 mice compared to SRG mice. Both human NK cell subsets, CD56^{bright}CD16⁻ and CD56^{dim}CD16⁺, were found to be present in the blood, spleen and liver of SRG-15 mice, as shown in FIG. 6D (spleen) (and data not shown). In addition, as shown in FIG. 6D, analysis of the two human NK cell subsets in the spleen of SRG-15 mice (mouse 1) showed that they had a distinct expression level of killer inhibitory receptors, with the CD56^{dim}CD16⁺ NK cell population including the higher percentage of CD158-expressing cells. This resembles what is found for NK cell subsets in the blood of humans (data not shown).

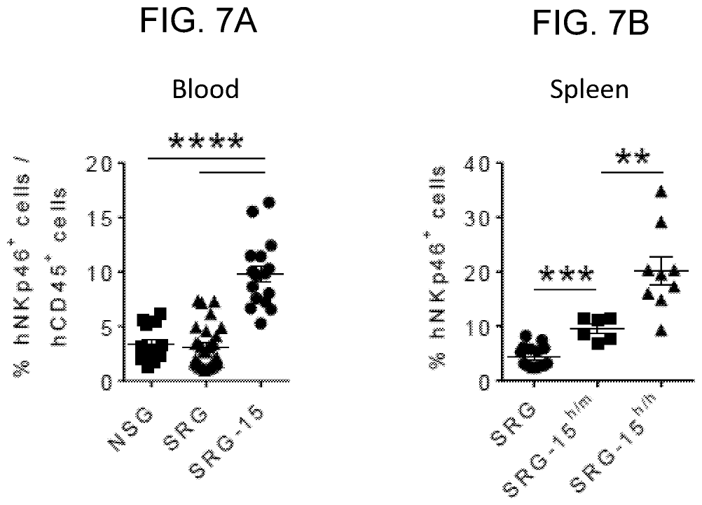

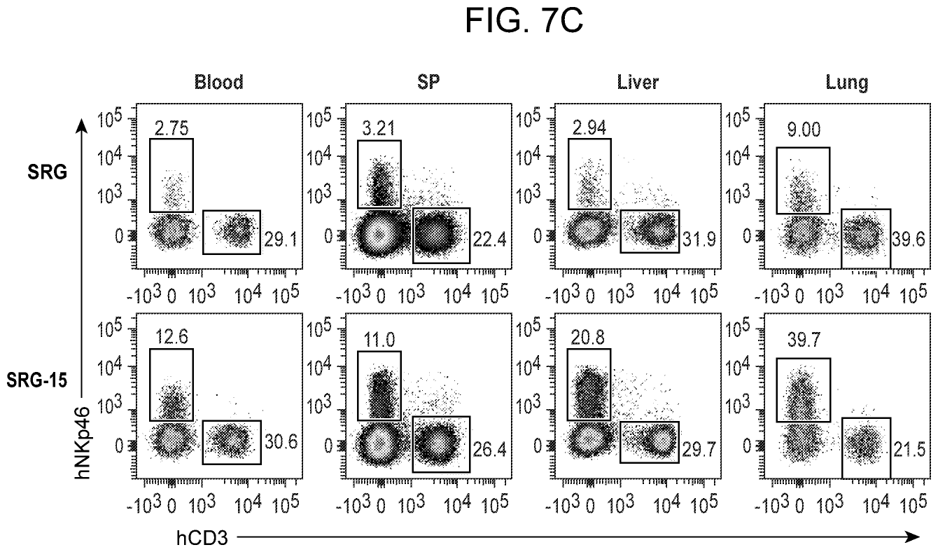

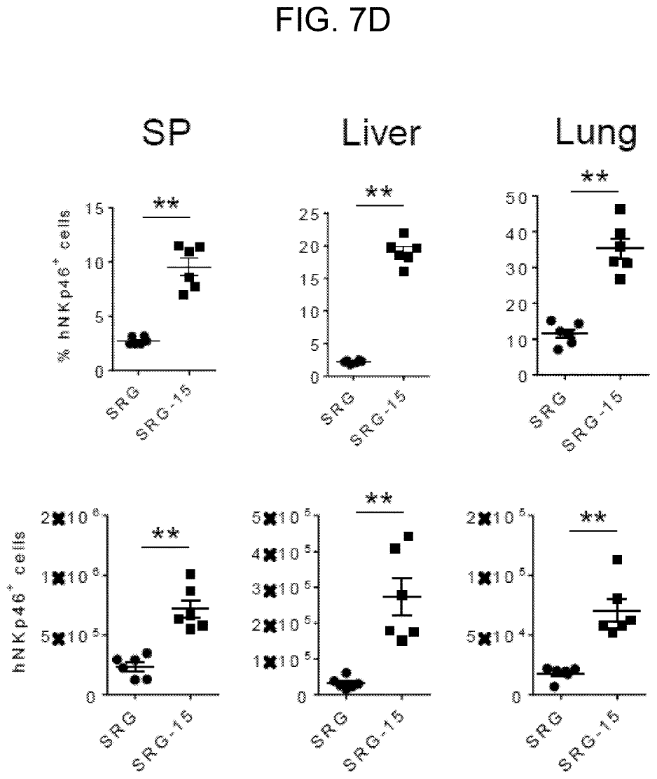

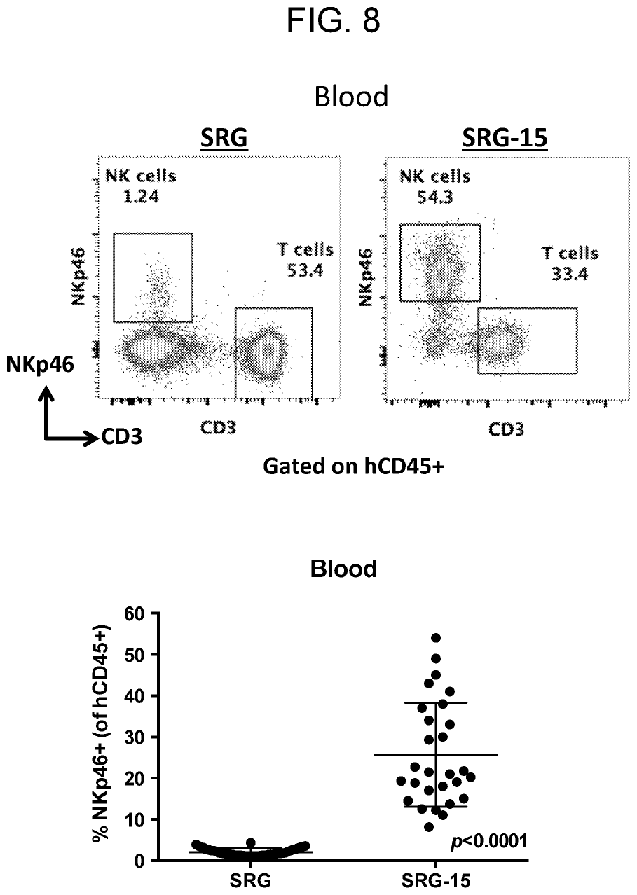

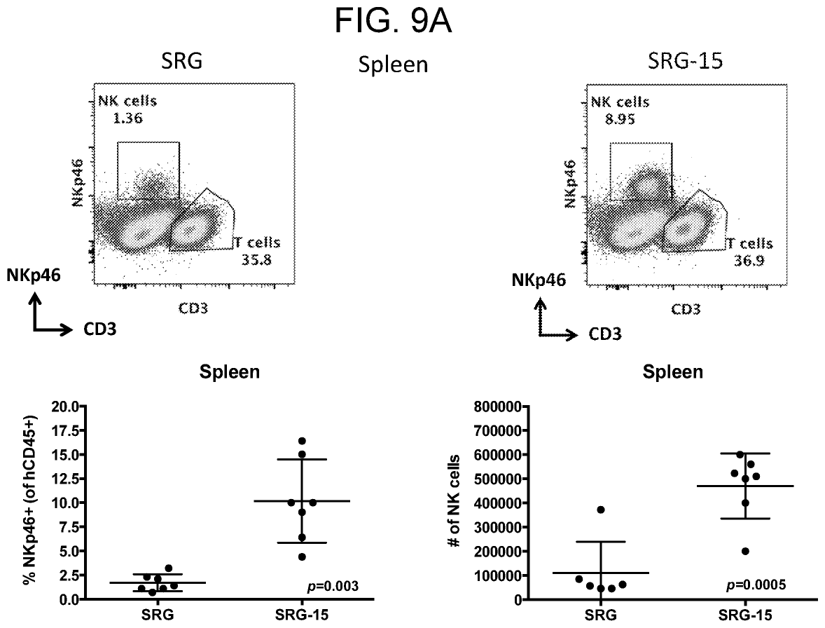

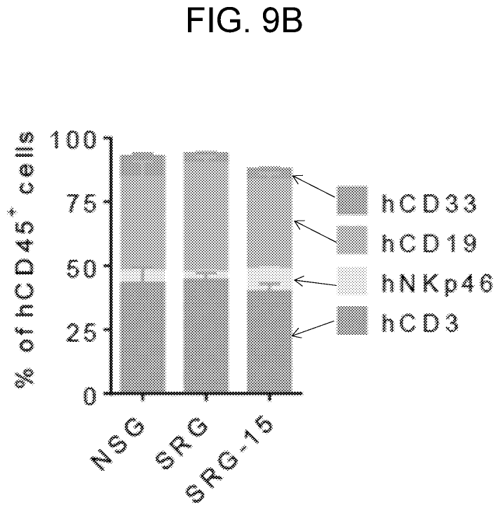

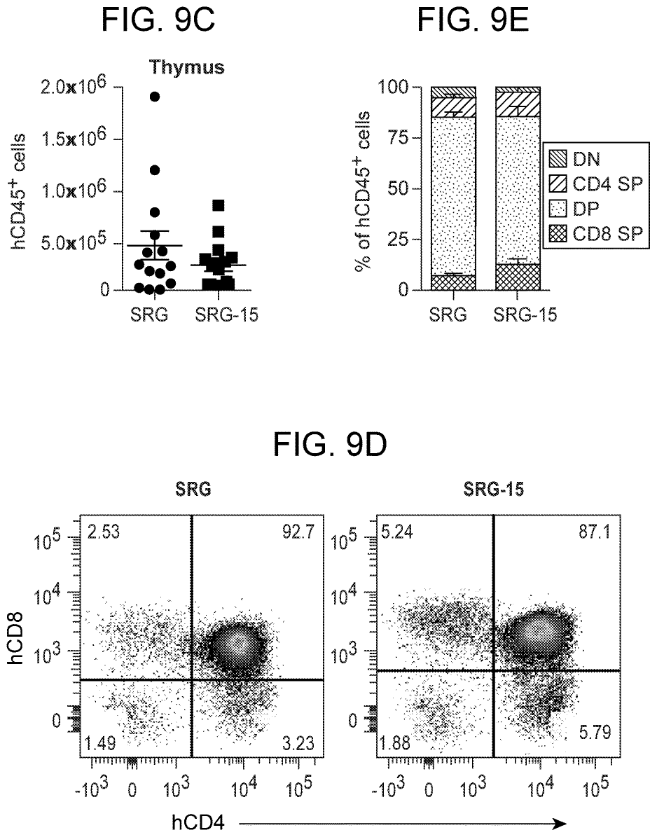

For SRG-15 mouse 2, efficient human NK cell engraftment in lymphoid and non-lymphoid tissues was seen as shown in FIGS. 7A-7D. FIGS. 7A and 7B show percentage of NK cells in blood and spleen, respectively. FIGS. 7C and 7D show the the frequency of human NK cells in the blood, spleen (SP), liver and lung of SRG and SRG-15 (mouse 2) mice 14 weeks post engraftment. Additional data showing NK cell distribution and percentage in blood and spleen of SRG and SRG-15 (mouse 2) mice from different experiments is provided in FIGS. 8 and 9A respectively. An increase in the hNKp46 fragment of hCD45+ cells in the blood of SRG-15 mice (mouse 2) is shown in FIG. 9B. FIGS. 9C-9E show relative numbers, distribution and composition of hCD45+ cells in the thymus of SRG and SRG-15 (mouse 2) mice.

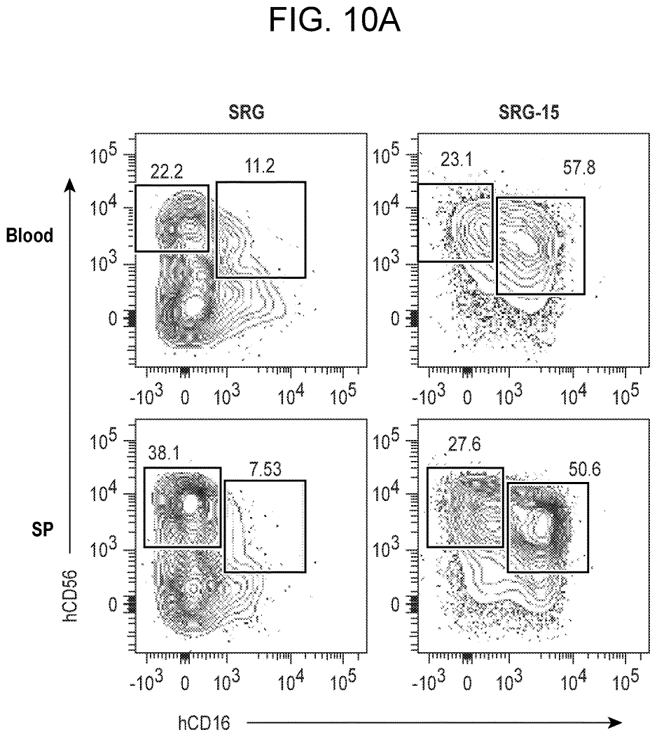

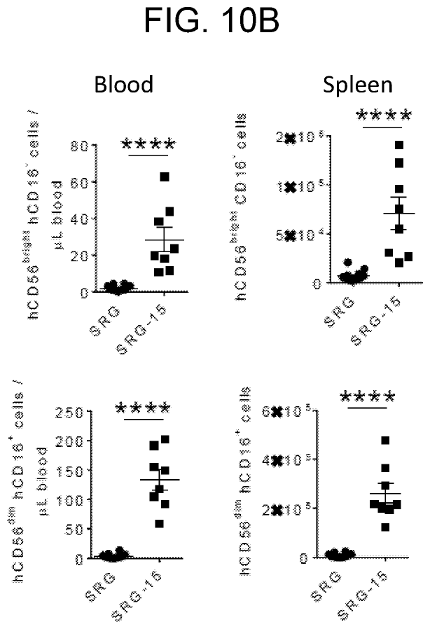

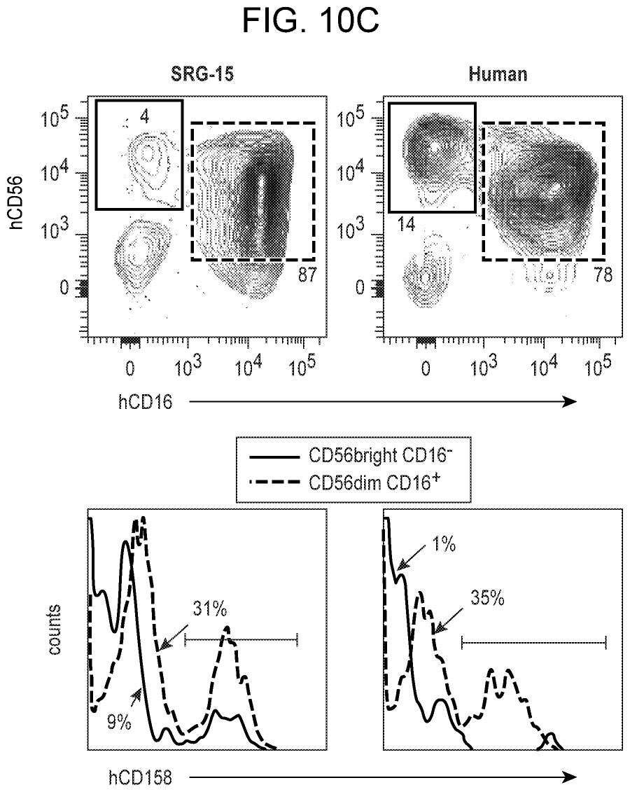

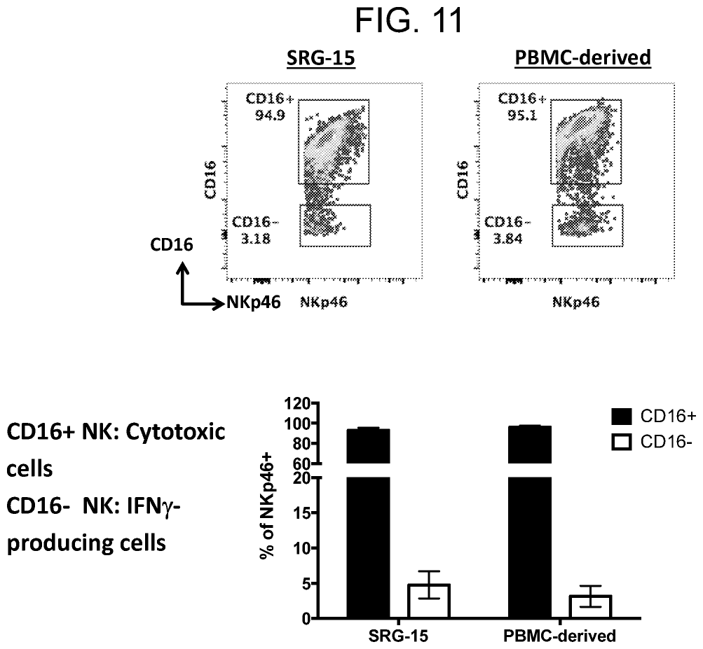

The NK cell subsets in humans and SRG-15 mice (mouse 2) were characterized. As shown in FIGS. 10A and 10B, increased levels of both hCD-56^{bright} hCD16⁻ and hCD56^{dim} hCD16⁺ were seen in the blood and spleen of SRG-15 mice relative to SRG mice. As in human, expression of killer inhibitory receptors (KIRs) was seen on NK cell subsets in SRG-15 mice (mouse 2) (FIG. 10C). FIG. 10C shows CD56bright CD16⁻ NK cells (left box for each plot) and CD56dim CD16⁺ NK cells (right box for each plot). The histogram below shows CD158 expression in those subsets. CD158 (KIR2D) on NK cell subsets in SRG-15 mice is similar to what is observed in human PBMC-derived NK cells.

Human NK cell distribution in the blood of SRG-15 mice was compared to that of blood obtained from two healthy human donors. Peripheral blood mononuclear cells (PBMCs) were isolated from buffy coats of two individual donors (obtained from BioreclamationIVT, Westbury, NY) over Ficoll-Paque; although greater percentage of blood NK cells was observed in engrafted SRG-15 mice than in PBMCs from human donors, a physiologically comparable distribution of cytotoxic (CD16+) NK cells versus IFN-g producing (CD16⁻) NK cells was observed (FIG. 11).

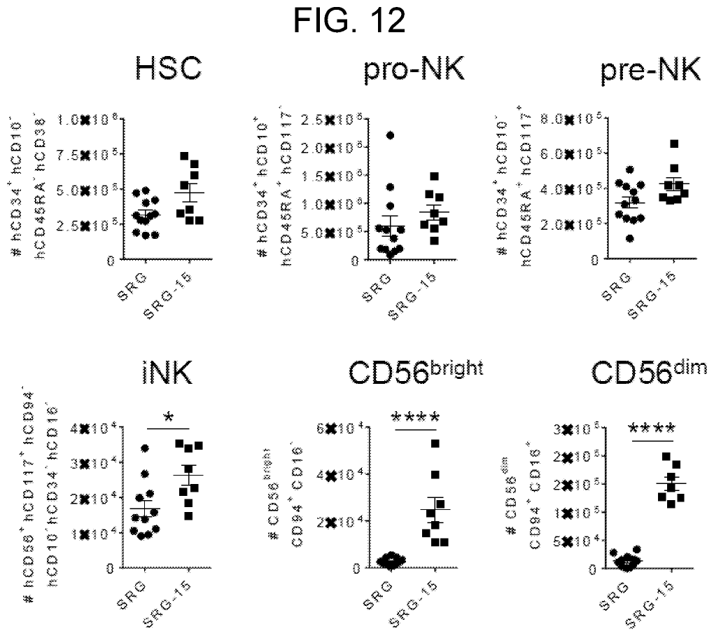

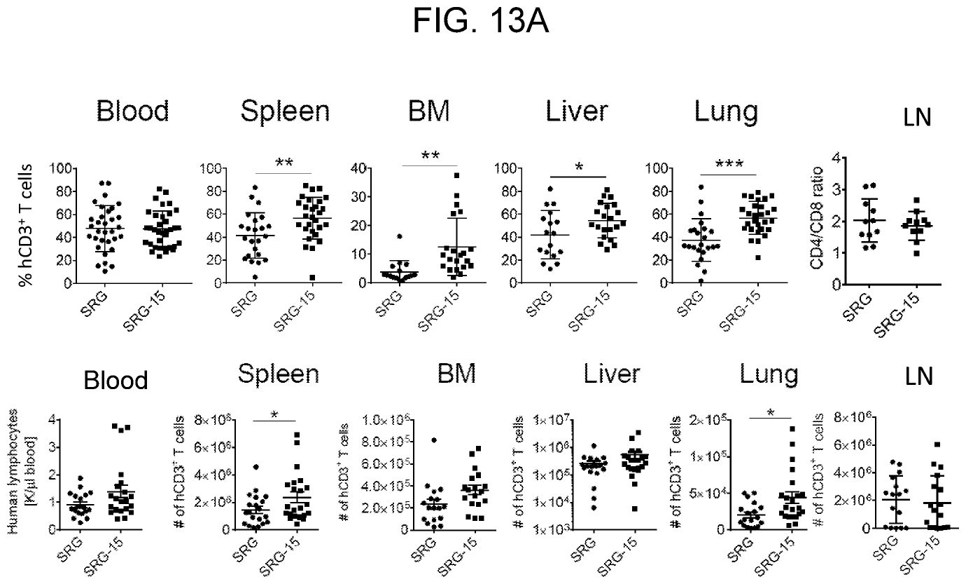

Finally, an analysis of the bone marrow of SRG and SRG-15 (mouse 2) showed increased human NK cell development in SRG-15 mice relative to SRG mice (FIG. 12).

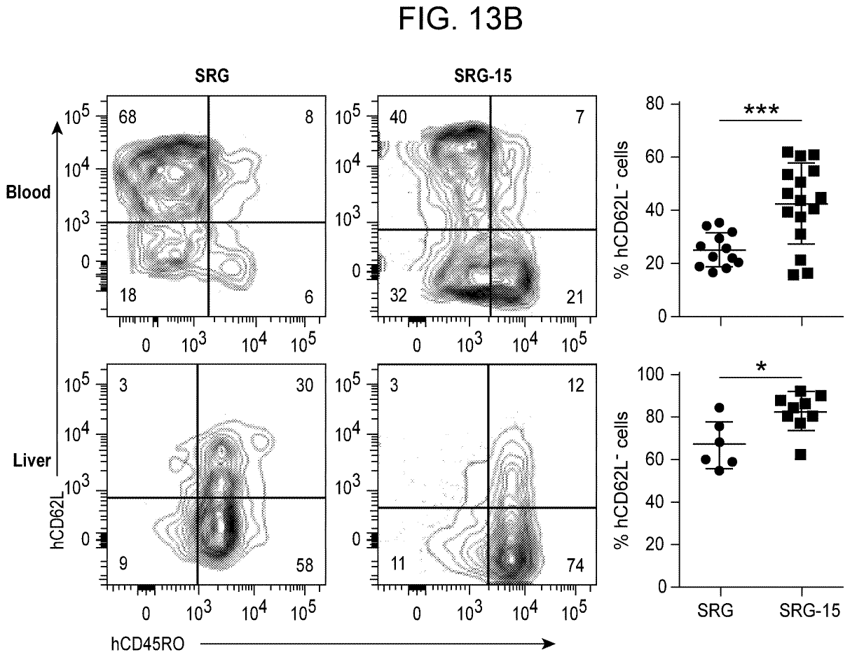

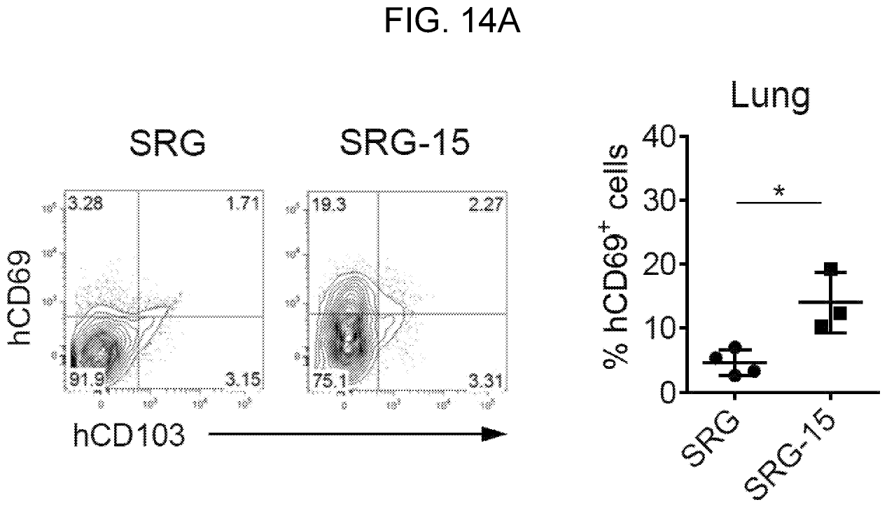

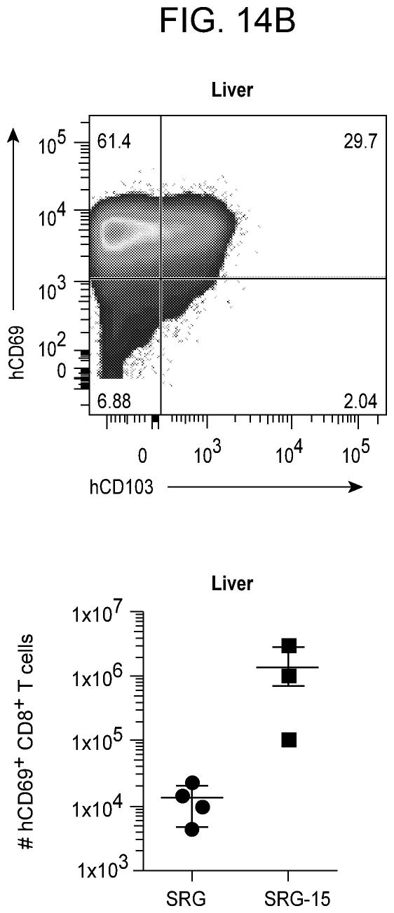

The impact of human IL-15 on human T cell development in SRG-15 mice was also assessed. A comparison of SRG-15 (mouse 1) mice relative to SRG mice showed that the effect of human IL-15 on the percentage, number and/or ratio of T cells varied depending on the tissue (FIG. 13A). The size and number of lymph nodes at week 16 post engraftment did not differ between SRG and SRG-15 mice, confirming the results that the numbers of human T cells in the lymph nodes of SRG and SRG-15 (mouse 1) mice were similar (FIG. 13A). FIG. 13B shows a human CD8⁺ T cell phenotype in blood and liver for SRG and SRG-15 mice (mouse 1), with an increase in hCD62L⁻ cells in SRG-15 mice (mouse 1) relative to SRG mice for both blood and liver. Additional data characterizing the T cells of the SRG-15 mice (mouse 1) relative to the SRG mice is provided in FIGS. 14A and 14B, which shows expression of the tissue-resident marker CD69 in the CD8⁺ T cells of lung (14A) and liver (14B) of SRG and SRG-15 mice. The above data provides evidence of an increase in effector tissue-resident T cells in SRG-15 mice.

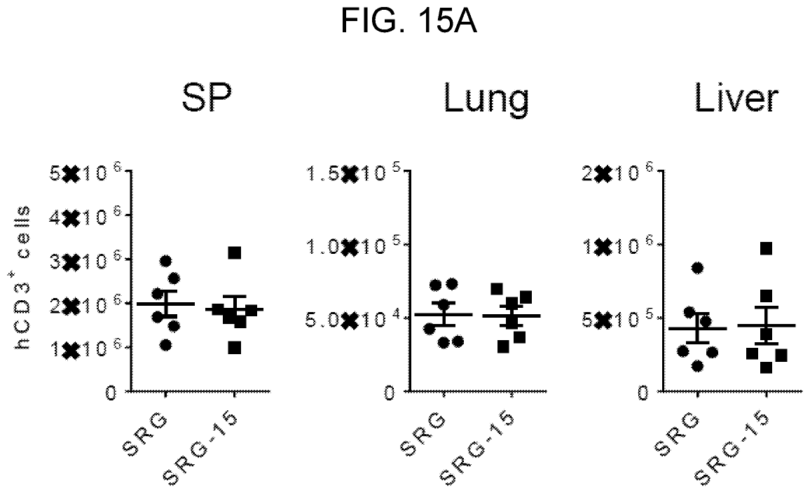

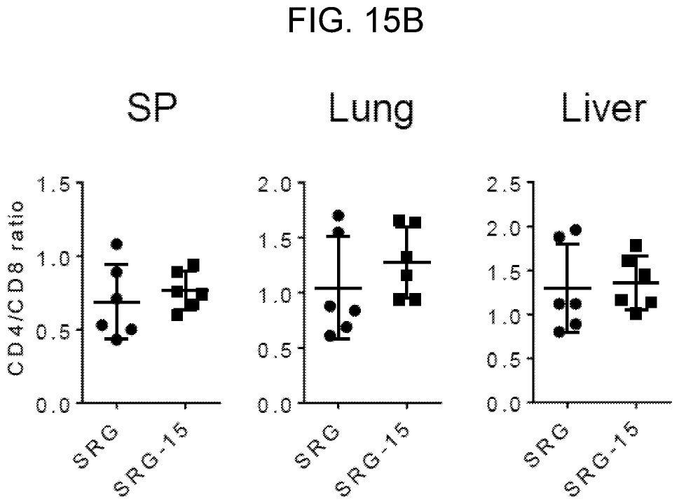

For mouse 2, the frequency of hCD3⁺ T cells in the spleen, lung and liver relative to SRG mice was assessed 16 weeks post engraftment, as shown in FIGS. 15A and 15B.

### Example 4: Development of Human Tissue-Resident Lymphocytes in SRG-15 Mice

Because IL-15 has been shown to be produced by epithelial cells in the gut and the lung and may play an important role for the development and survival of human tissue-resident T and NK cells, human tissue-resident T and NK cells were analyzed in SRG and SRG-15 mice.

### Materials and Methods

Neonate mice are irradiated sub-lethally without anesthesia 3-5 days post birth with 160cGy and returned to their mothers for rest. 4-12 hours post irradiation these neonates are transplanted with CD34+ huHSCs in 25 μl PBS intrahepatically (i.h.) using a 30G needle.

### Results

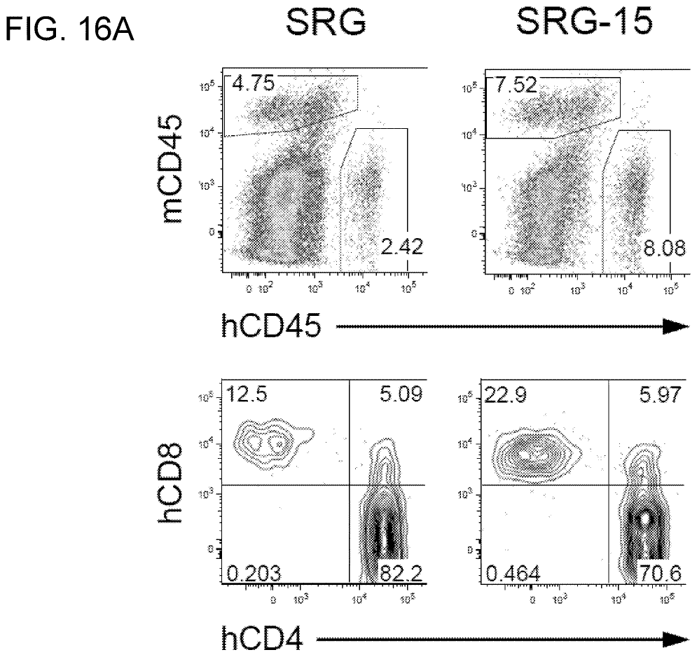

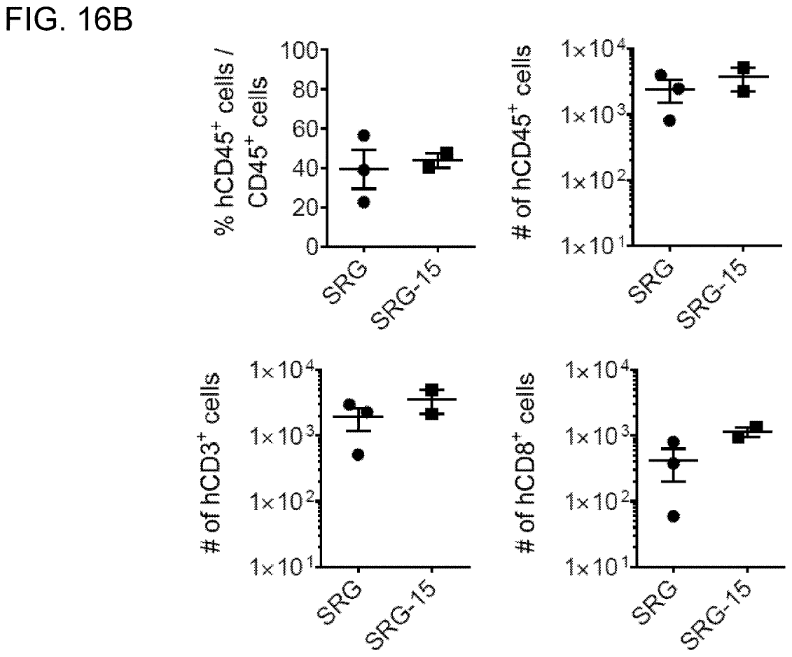

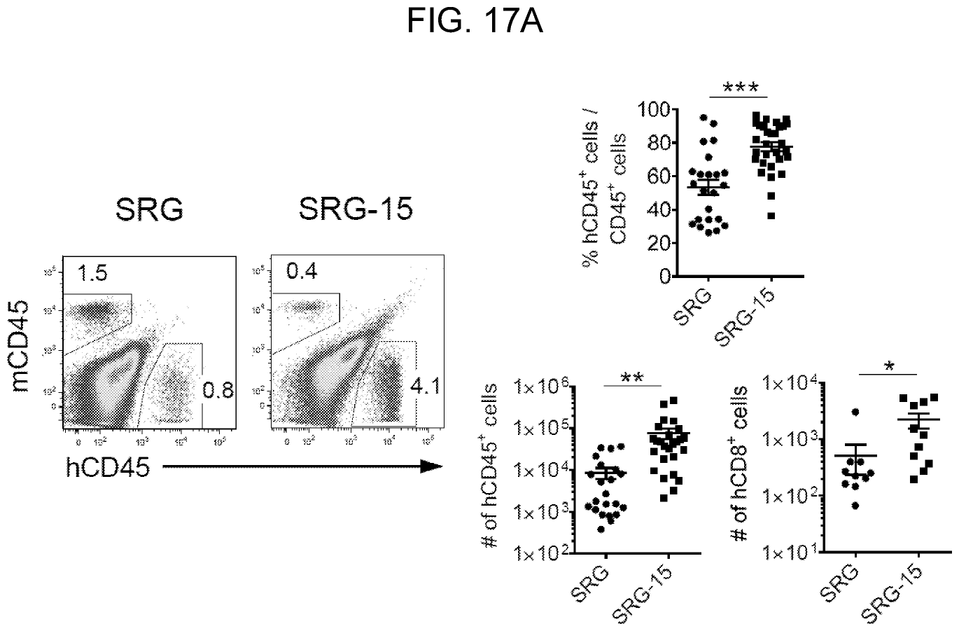

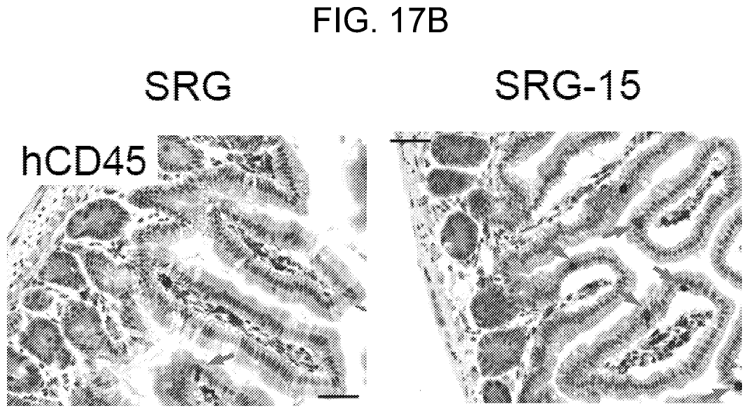

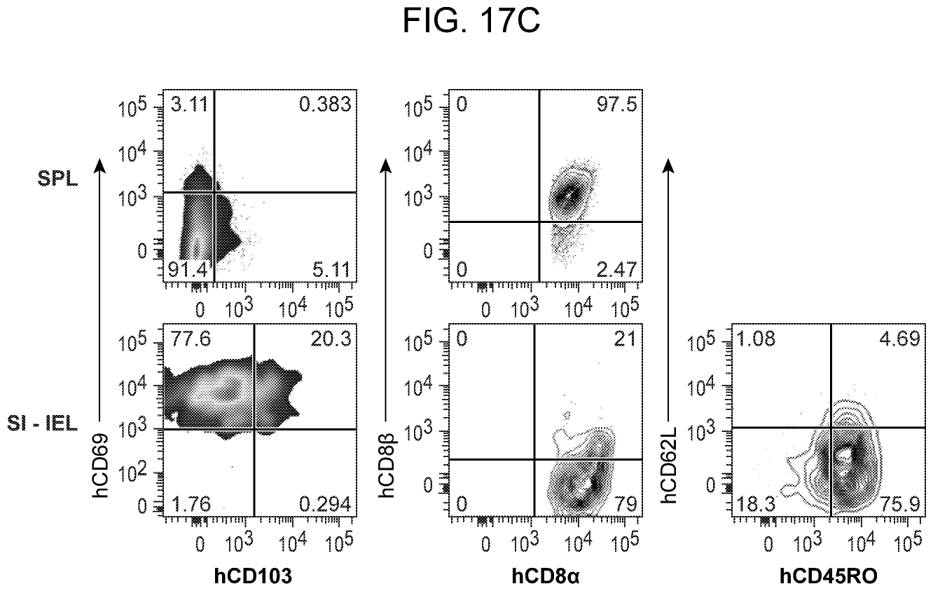

As shown in FIG. 17A, isolation of the intraepithelial lymphocyte population from the small intestine during steady state conditions in mouse 1 revealed a higher frequency of human CD45⁺ cells in SRG-15 mice compared to SRG mice. Immunohistochemical analysis, as illustrated in FIG. 17B, demonstrated that the human CD45⁺ NK cells were located in the epithelial cell layer of the small intestine of SRG-15 mice (mouse 1) (as designated by the arrows in FIG. 17B), while very few intraepithelial lymphocytes were found in SRG mice. Human CD8⁺ IELs in SRG-15 mice showed high expression of CD69, the typical marker of tissue-resident T cells. In contrast to human IELs (Sathaliyawala T, Kubota M, Yudanin N et al. Immunity 2013; 38:187-197), only a subpopulation of human CD8⁺ IELs in the SRG-15 mice expressed the tissue-resident marker CD103 (FIG. 17C). As shown in FIGS. 16A and 16B, the phenotype of increased human CD8⁺ IELs in SRG-15 mice (mouse 1) was specific as there was little difference in the number of lamina propria cells in the colon during steady state between SRG and SRG-15 mice. In addition to the increased number of human T cells in the lung of SRG-15 mouse 1 as shown in FIG. 13A, higher expression of CD69 on human CD8⁺ T cells in the lung of SRG-15 mice compared to SRG mice was also found as shown by FIG. 14A. In addition, FIG. 14B shows a higher level of hCD69 expressing CD8⁺ T cells in the liver of SRG-15 mouse 1 compared to the SRG mouse.

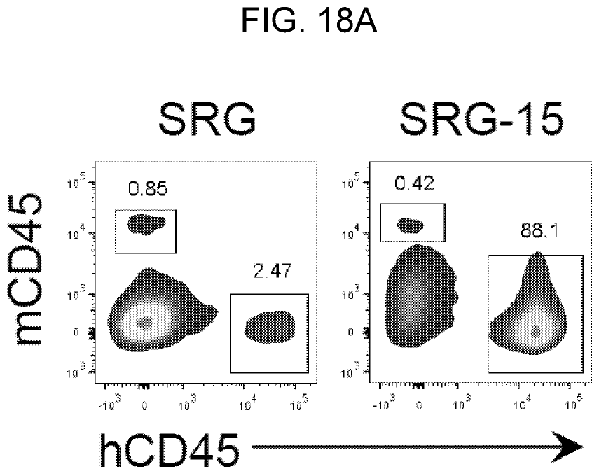

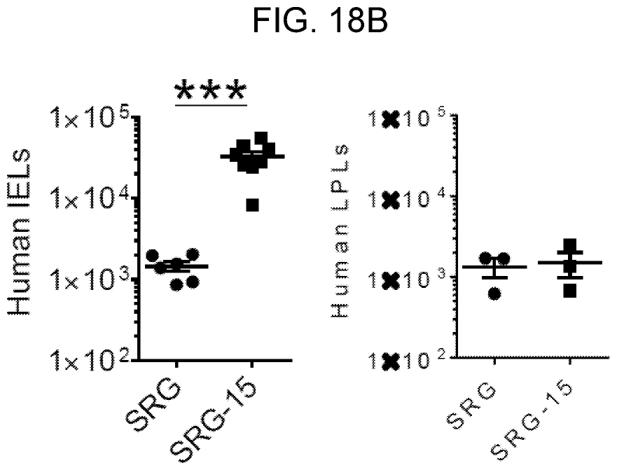

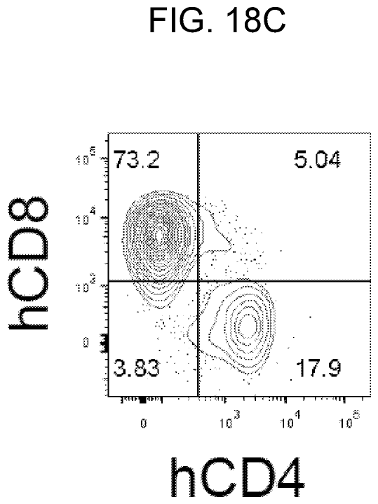

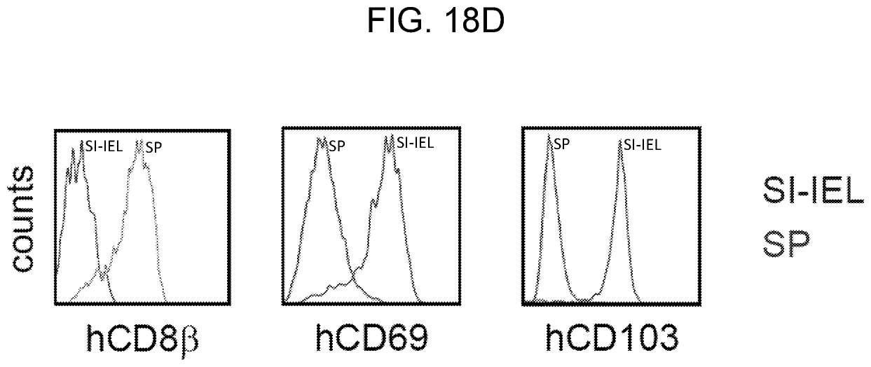

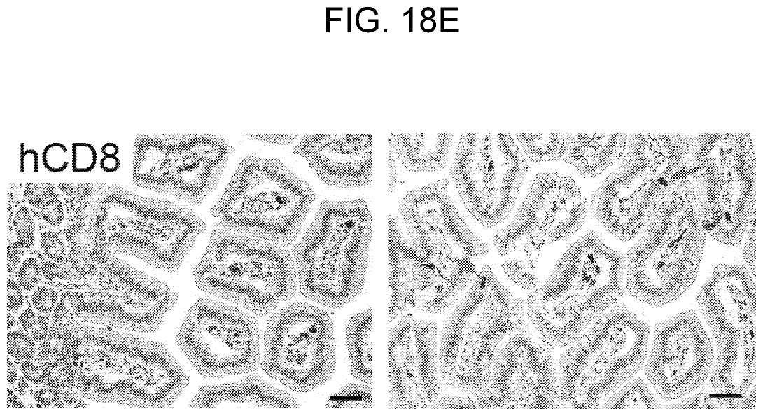

Similar to the SRG-15 engrafted mouse 1, in SRG-15 engrafted mouse 2, FACS analysis revealed a higher proportion of human CD45⁺ cells in the IEL fraction of SRG-15 mice compared to SRG mice (FIG. 18A). In addition, while the number of LPLs was not significantly changed between SRG and SRG-15 (mouse 2) mice, a significant increase in IELs was seen in SRG-15 (mouse 2) mice relative to SRG mice (FIG. 18B). The composition of hCD3+ cells in the small intestine of SRG-15 mice (mouse 2) is provided in FIG. 18C and shows a greater proportion of hCD8+ relative to hCD4+ cells. The phenotypic characteristics of hCD3+ hCD8+ T cells in the spleen and small intestine of SRG-15 mice (mouse 2) are provided in FIG. 18D. Immunohistochemical analysis, as illustrated in FIG. 18E, demonstrated that the human CD8⁺ IELs were located in the epithelial cell layer of the small intestine of SRG-15 mice (mouse 2) (as designated by the arrows in FIG. 18E), while very few intraepithelial lymphocytes were found in SRG mice.

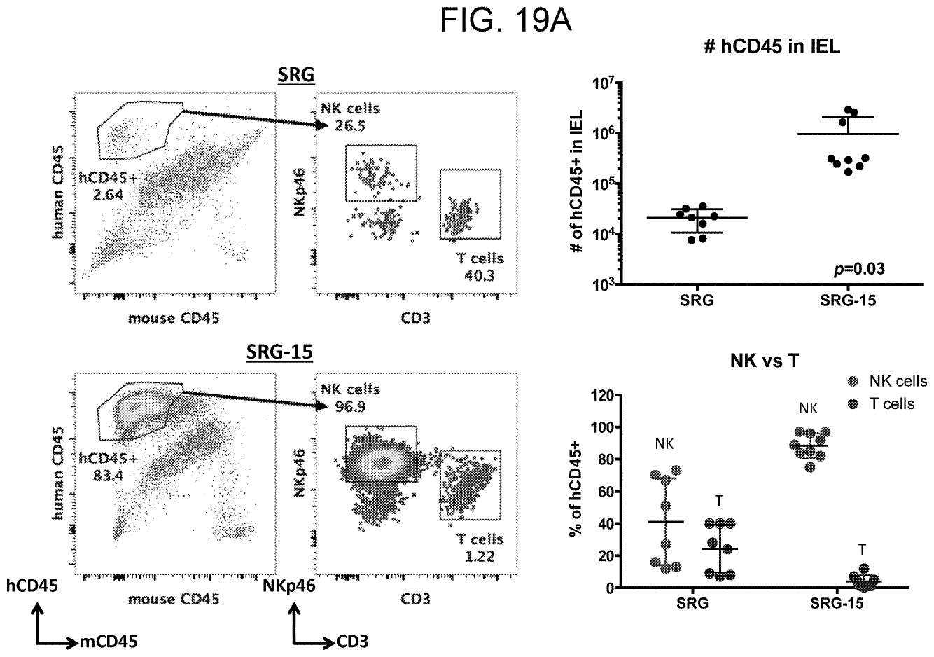

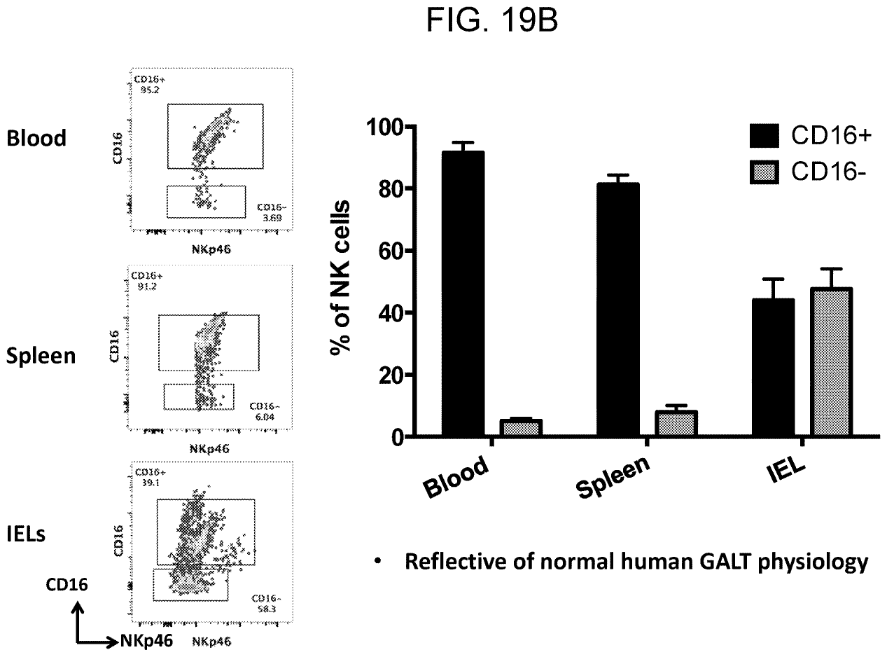

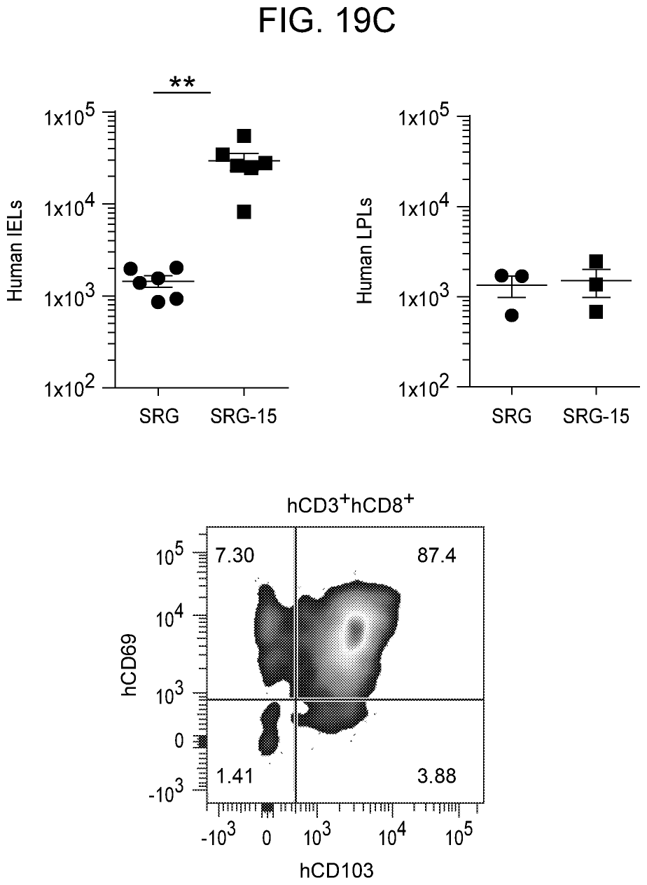

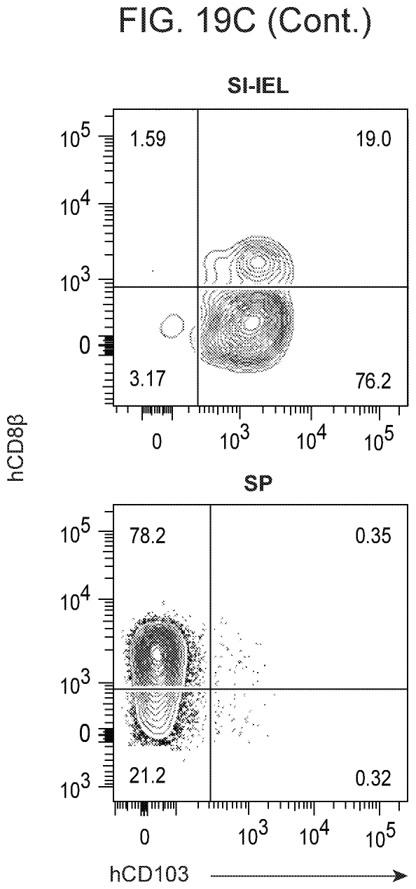

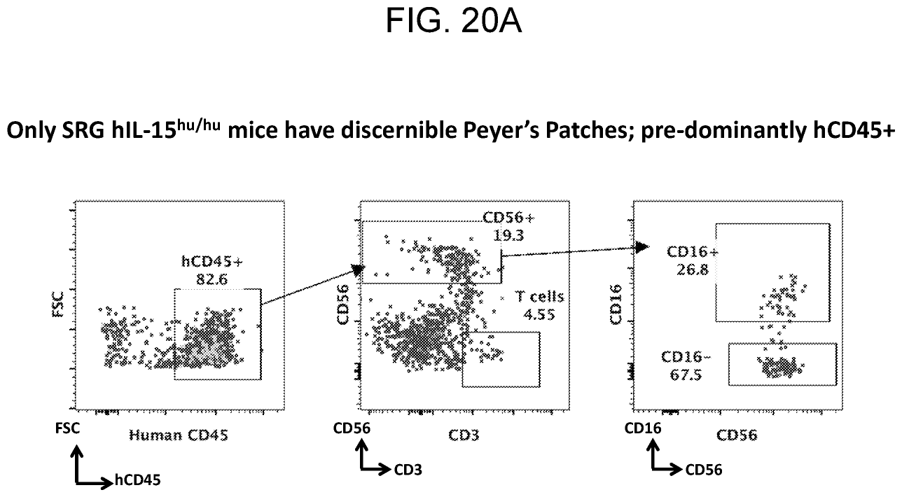

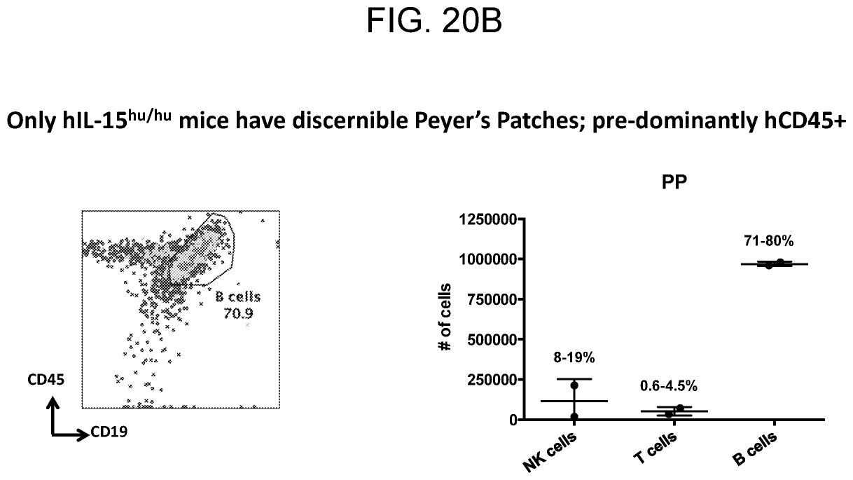

As discussed above with respect to FIGS. 18A and 18B, in SRG-15 engrafted mouse 2, greater gut-associated lymphoid tissue (GALT) resident intraepithelial human lymphocyte reconstitution (IELs) was observed compared to SRG mice (FIGS. 19A and 19C). Interestingly, the majority of the human lymphocytes observed were human NK cells. As expected for normal human GALT physiology, the majority of of NK cells in the SRG-15 mouse 2 in both blood and spleen were cytotoxic NK cells (CD16+), while in IEL, there was a comparable distribution of CD16+ versus CD16⁻ NK cells (FIG. 19B). There were no changes in the number of lamina propria lymphocytes between the engrafted SRG and SRG-15 mice (FIG. 19C). Unlike engrafted SRG-15 mouse 1, in engrafted SRG-15 mouse 2 a greater proportion of human CD3+ CD8+ IELs expressed human CD103 marker. Peyer's patches were completely absent in SRG mice but they were present in the SRG-15 mouse 2 and were populated with human lymphocytes as shown in FIGS. 20A and 20B.

### Example 5: Determining the Functional Role of Human Tissue-Resident T Cells in SRG-15 Mice During Viral Infections

To test whether tissue-resident T cells in SRG-15 mice have a functional relevance during homeostasis, it was determined whether the increased number of human CD8⁺ IELs in SRG-15 mice induces characteristic changes in the composition of the mouse gut microbiota.

### Materials and Methods

Neonate mice are irradiated sub-lethally without anesthesia 3-5 days post birth with 160cGy and returned to their mothers for rest. 4-12 hours post irradiation these neonates are transplanted with CD34+ huHSCs in 25 μl PBS intrahepatically (i.h.) using a 30G needle.

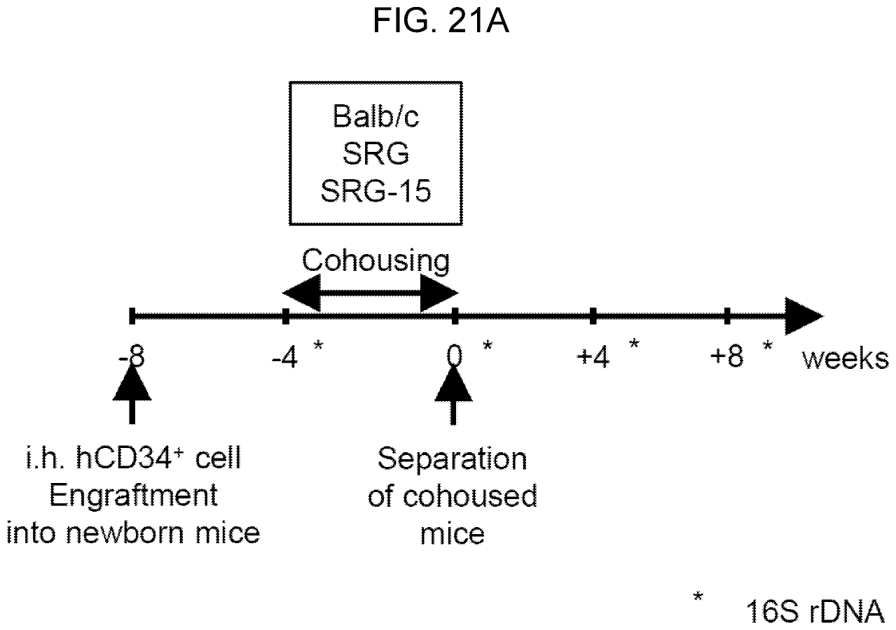

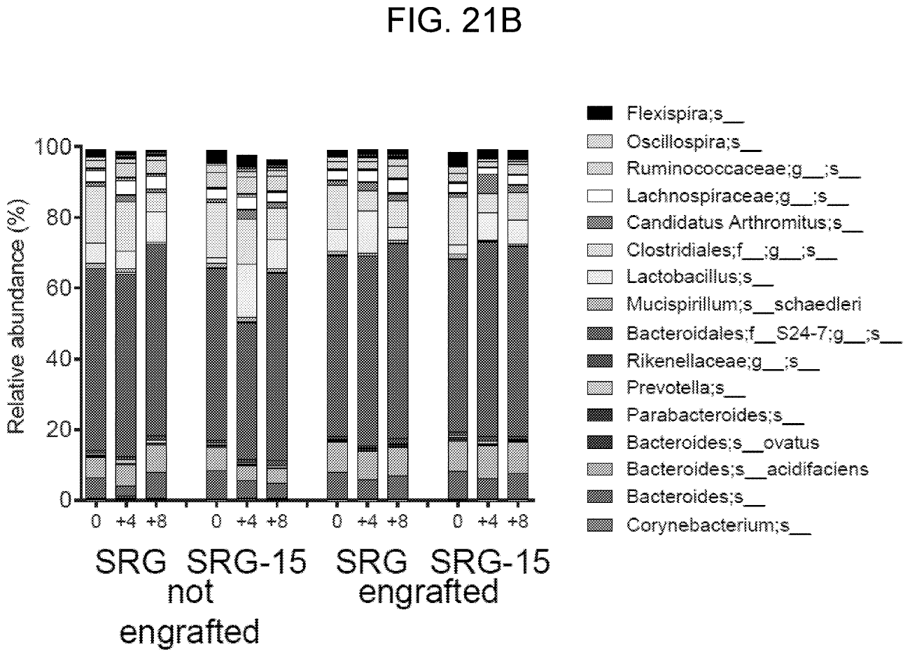

Four weeks post engraftment, SRG-15 mice were cohoused for four weeks with SRG and donor Balb/c mice to equalize the gut microbiota between the different strains. The mice were then separated and fecal samples were collected and analyzed by 16S rRNA sequencing. FIG. 21A provides a timeline for cohousing and feces sample collection for gut microbiota sequencing.

### Results

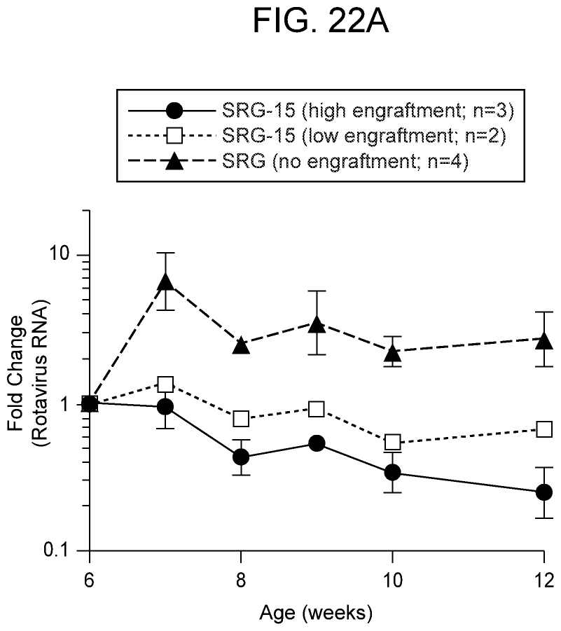

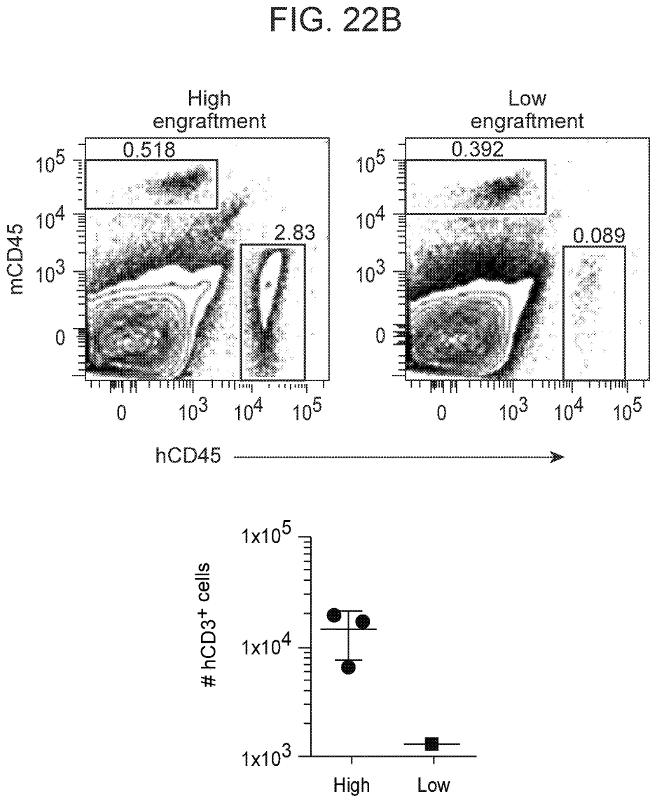

As illustrated in 21B, for mouse 1, the results show that there were no significant changes between engrafted SRG-15 and SRG mice after cohousing, indicating that the developing human CD8⁺ IELs do not induce major changes during steady state conditions. Additional experiments were conducted to determine whether CD8⁺ IELs, which are sufficient to clear acute rotavirus infection, can clear rotavirus infection in engrafted SRG-15 mice. As shown in FIG. 22, the results indicated that acute rotavirus infection can be cleared in engrafted SRG-15 mice but not in non-engrafted SRG mice.

### Example 6: Analysis of NK Cell Subsets in SRG-15 Mice (Mouse 2) and Humans

NK cell subsets in SRG-15 (mouse 2) mice were characterized for various phenotypic markers and compared with humans.

### Materials and Methods

NK cell subsets were detected via Cytometry by Time-of-Flight (CyTOF), as described generally in Yao et al. J. of Immunological Methods 415 (2014) 1-5, and analyzed using ViSNE (el-AD et al. Nat. Biotechnol. 2013 June; 31(6):545-52doi: 10.1038/nbt.2594. Epub 2013 May 19.

### Results

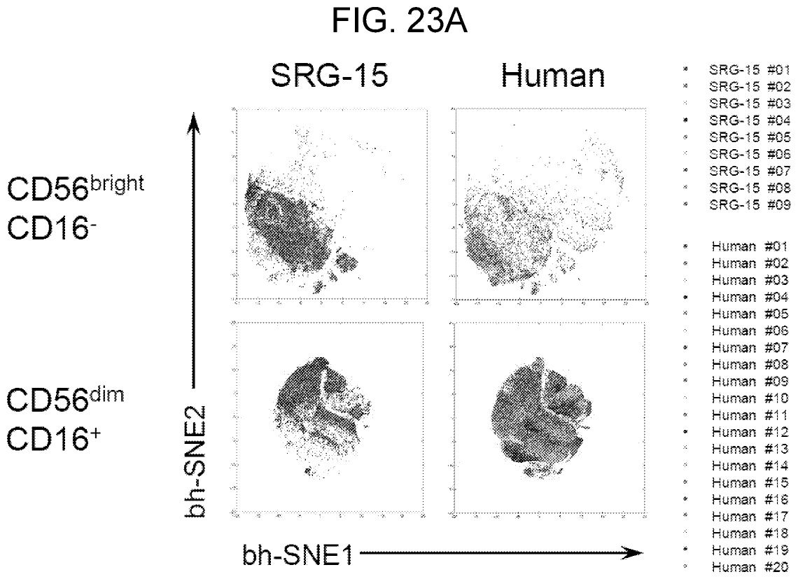

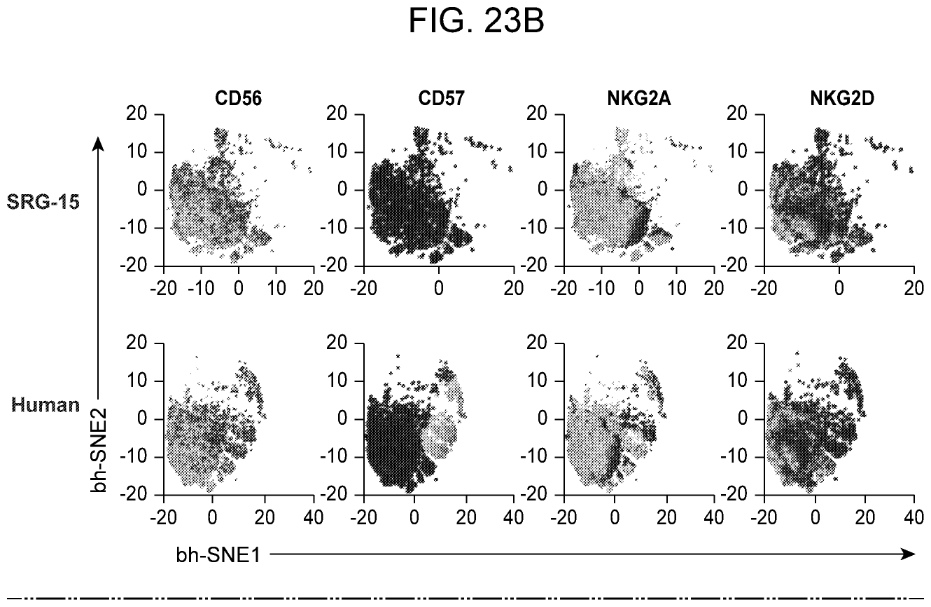

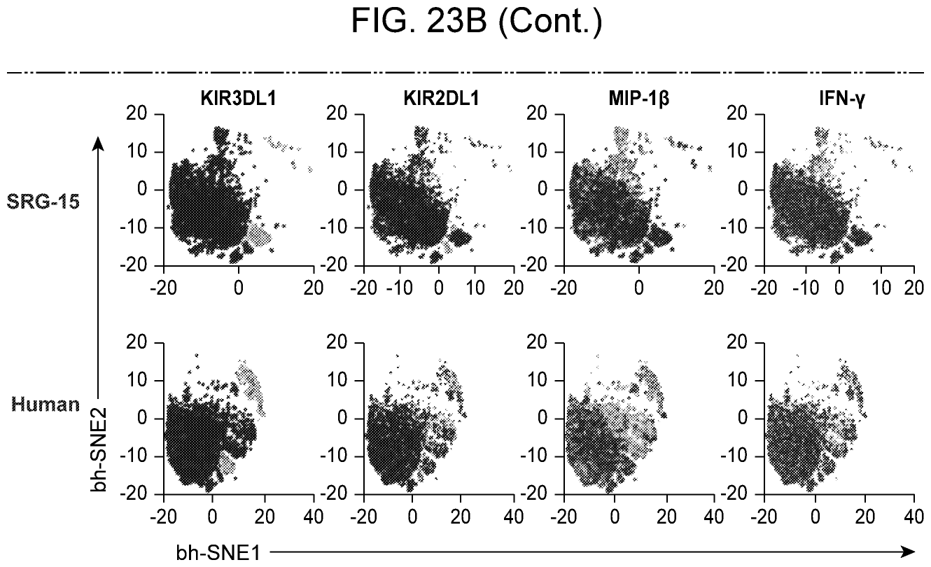

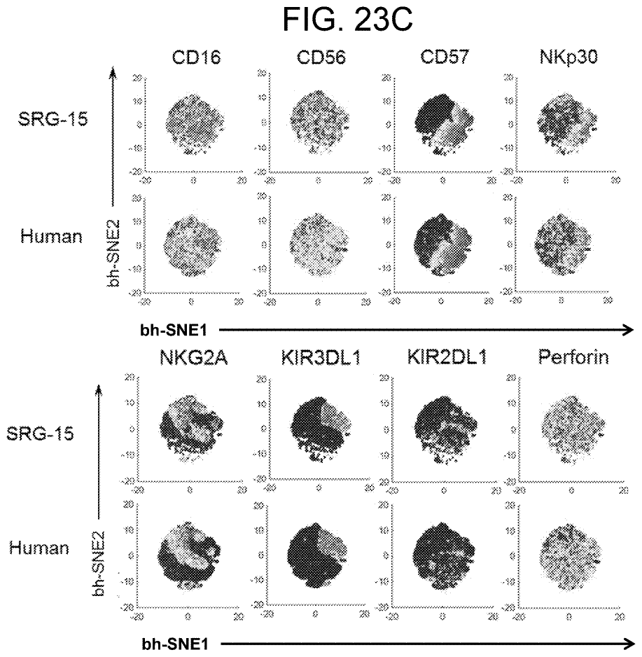

FIG. 23A provides ViSNE plots showing CyTOF-based analysis of 33 parameters of CD56^{bright} CD16⁻ and CD56^{dim} CD16⁺ NK cell subsets in humans (n=20) and SRG-15 mice (mouse 2) (n=9). Each dot represents a single cell.

FIG. 23B provides ViSNE plots showing the expression intensity of eight selected markers on CD56^{bright} CD16⁻ NK cells in humans (n=20) and SRG-15 mice (mouse 2) (n=9).

FIG. 23C ViSNE plots showing the expression intensity of eight selected markers on CD56^{dim} CD16⁺ NK cells in humans (n=20) and SRG-15 mice (n=9). This multi-dimensional single-cell analysis of 33 key molecules of human NK cells indicate that the human NK cells that develop in SRG-15 mice are highly comparable to human NK cells in healthy individuals.

### Example 7: Cytotoxic Capacity of NK Cells from SRG-15 mice

### Materials and Methods

For in vitro NK cytotoxicity studies, isolated splenic NK cells from human HSC-engrafted SRG and SRG-15 mice (mouse 2) were treated overnight with human IL-2. The next day, NK cells were cultured with CFSE-labeled, NK-susceptible K562 target cells at varying effector to target ratios (E:T). After 5 hr co-culture, killing of K562 cells was measured by FACS analysis of viability dye Topro3 uptake by K562 cells (gated on CFSE+ cells to distinguish K562 and then analysis of percent positive for Topro3).

Additionally, for in vitro antibody-dependent cellular cytotoxicity (ADCC) studies, isolated splenic NK cells from human HSC-engrafted SRG and SRG-15 mice were treated overnight with human IL-2. The next day, NK cells were cultured with CFSE-labeled Raji target cells at varying effector to target ratios (E:T). Raji cells were pre-treated with anti-CD20 (Rituximab) or control IgG. After 5 hr co-culture, killing of Raji cells was measured by FACS analysis of viability dye Topro3 uptake by Raji cells (gated on CFSE+ cells and then analysis of percent positive for Topro3).

For in vivo NK cell activation studies, human HSC-engrafted SRG and SRG-15 mice (mouse 2) were injected intra-peritoneally with 50 μg poly IC. Mice were pre-bled (before poly IC injection) and 18 hours after poly IC injection. Human CD45+ NKp46+ (NK cells) were analyzed for activation marker CD69 expression by FACS pre- and post-poly IC administration.

### Results

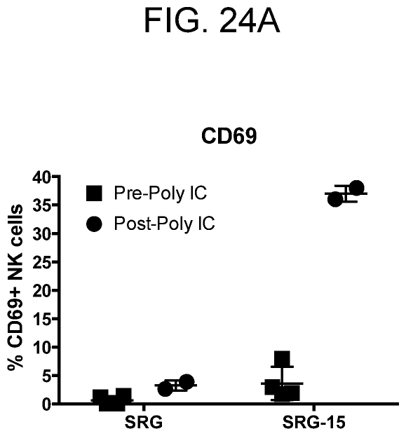

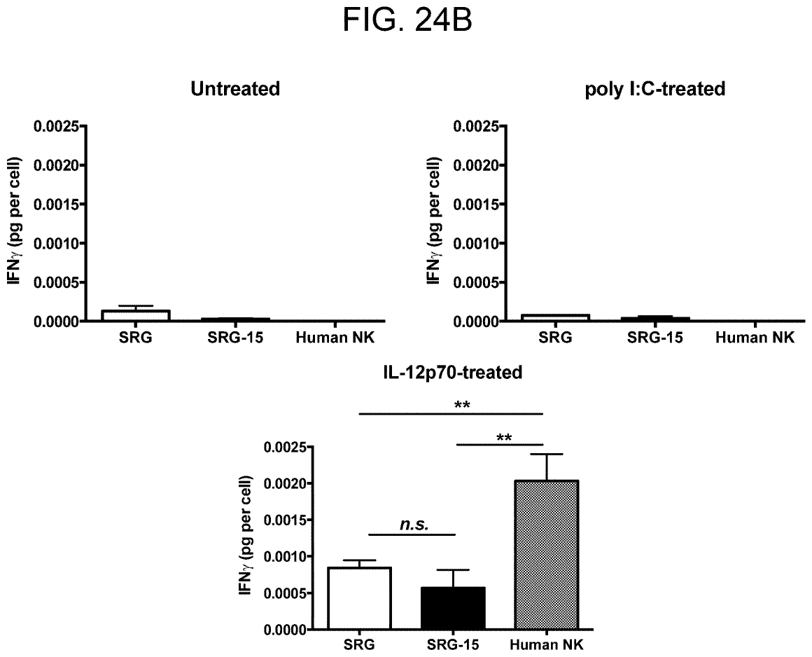

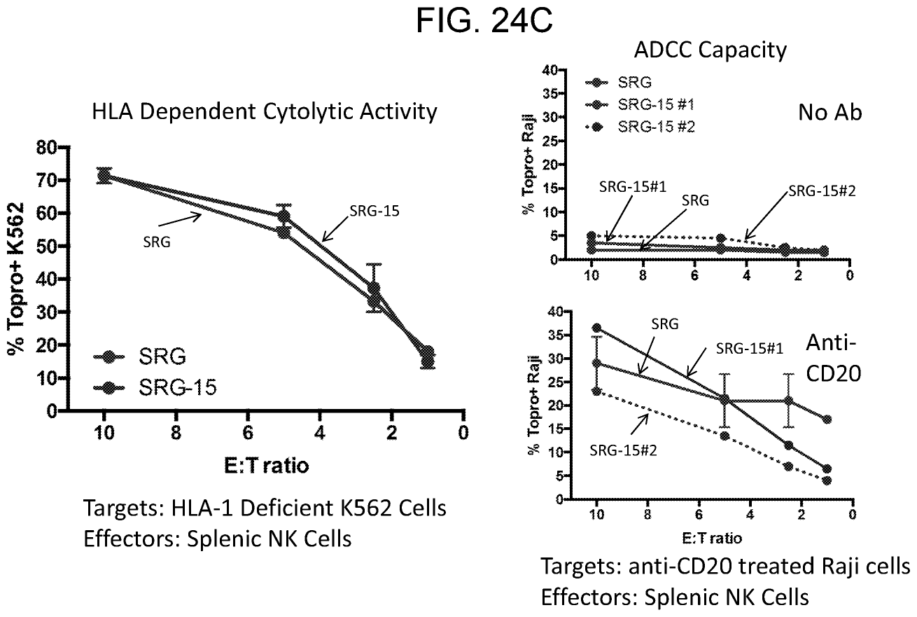

In a classical NK cytotoxicity study, classical NK target HLA class I deficient K562 cells were subject to killing by activated NK cells from SRG or SRG-15 mice (mouse 2). As shown in FIG. 24C (left) splenic NK cells from SRG and SRG-15 mice showed comparable cytolytic capacity with respect to K562 cells when normalized for number.

NK cells are typically responsible for anti-CD20 antibody mediated ADCC against B cell leukemias and lymphomas (see, e.g., J. Golay et al. Haematologica 2003; 88:1002-12). In order to demonstrate the ability of NK cells from SRG-15 engrafted mice to facilitate anti-CD20 mediated ADCC, splenic NK cells from both SRG and SRG-15 mice were tested and shown to exhibit comparable antibody-dependent cellular toxicity (ADCC) activity against anti-CD20 treated Raji cells when normalized for cell number (FIG. 24C (right)).

As depicted, e.g., in FIGS. 8 and 9, there is a significant upregulation of NK cells in both spleen and blood of SRG-15 animals. The capacity for activation of NK cells in SRG-15 mice was tested by measuring CD69 marker activation after a poly-IC injection. As shown in FIG. 24A, the percentage of NK cells positive for the activation marker CD69 was increased in SRG-15 mice relative to SRG mice. As SRG-15 NK cells were shown to mediate ADCC comparable to SRG NK cells in vitro under normalized conditions, the ability of SRG-15 NK cells to exhibit a greater activated phenotype in vivo, as well as greater numbers of NK cells in SRG-15 mice, suggests that SRG-15 mice may be a suitable in vivo model to study human NK cell ADCC.

### Example 8: IFNγ Production from SRG and SRG-15 Derived NK Cells

### Materials and Methods

NK cells were isolated from pooled splenocytes of SRG or SRG-15 mice (3 spleens per group) and NK cells were isolated using EasySep Human NK enrichment kit (StemCell Technologies; Cat #19055).

NK cells were also isolated from healthy human PBMCs. NK cells were treated overnight with 10 ng/mL human IL-2. The next day, cells were stimulated overnight with 10 ng/ml human IL-12p70 or 2 mg/mL poly I:C or left untreated. The next day, supernatant was harvested and IFNg levels assessed using Human IFNg Quantikine ELISA kit (R&D systems; Cat #DIF50). NK cell purity was analyzed by FACS and IFNg levels normalized as picograms (pg) produced by individual NK cells. Statistical analysis was performed using ANOVA test.

### Results

As shown in FIG. 24B, SRG and SRG-15 derived NK cells have comparable IFNγ secretion, but less than human PBMC-derived NK cells upon IL-12p70 treatment.

### Example 9: Human NK Cells Inhibit Tumor Growth in SRG-15 Mice

The ability of human NK cells to infiltrate human tumor xenographs and inhibit tumor growth in SRG-15 mice (mouse 2) was tested.

### Materials and Methods

Rituximab was injected i.p. every other day (started at day 14 post s.c. injection of 5 million Raji cells). Tumor growth was assessed by caliper measurement and the volume was calculated using the following formula: tumor volume=0.5×(length×width\hat{ }2). Data were pooled from 2 independent experiments. Statistical analsysis was performed using unpaired, two-tailed Mann-Whitney U-test comparing engrafted, untreated SRG-15 and engrafted, RTX-treated SRG-15 mice (* P<0.05).

The s.c. tumor was crushed and digested using Collagenase D (1 hour, 37 C). The recovered cells, including tumor and immune cells were analyzed by an LSRII flow cytometer.

### Results

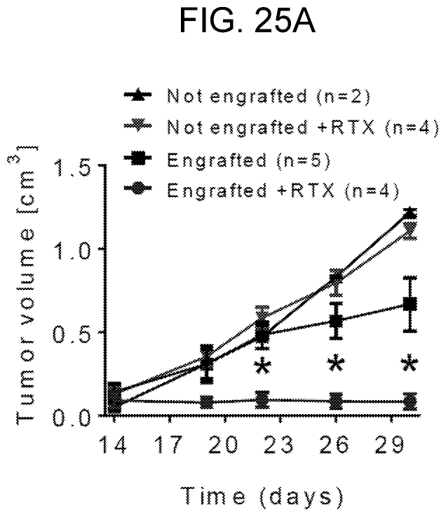

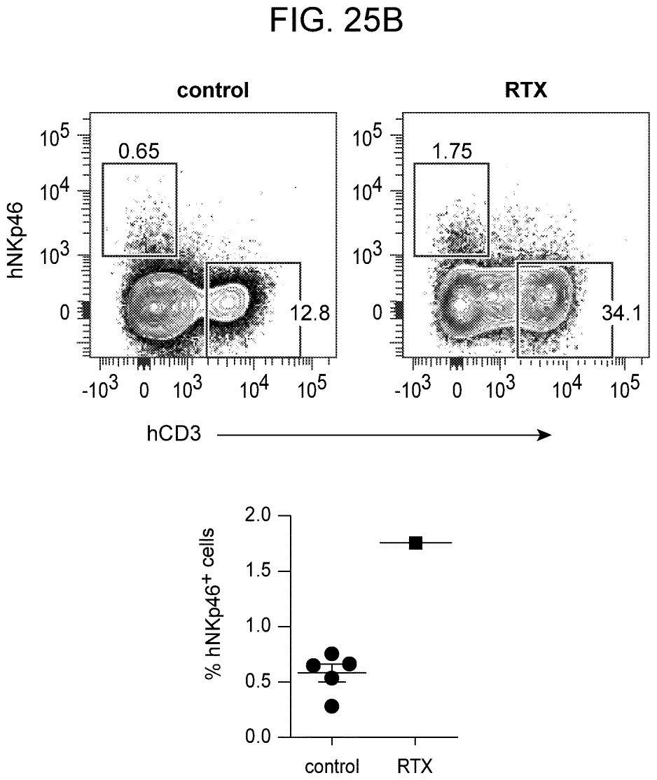

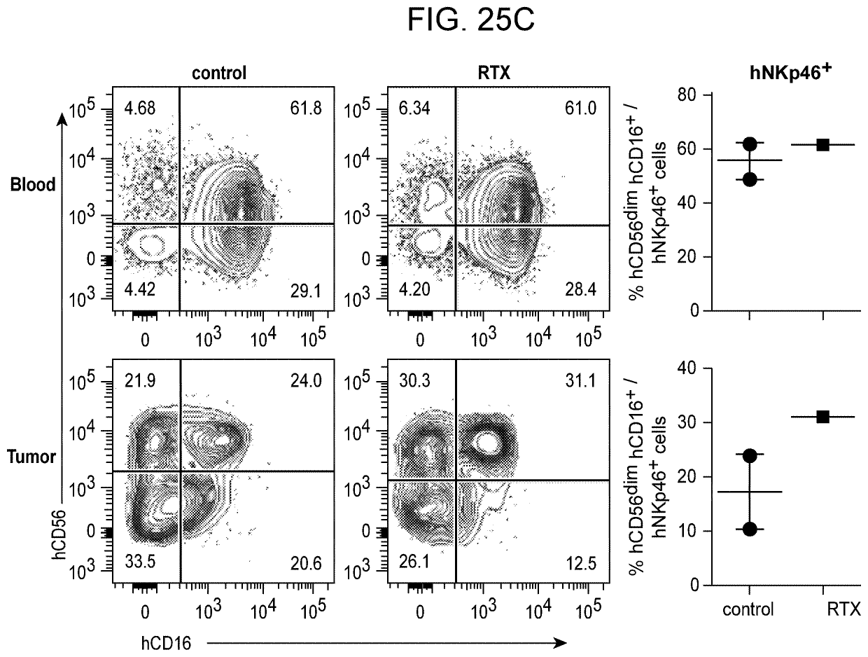

As shown in FIG. 25A, human NK cells in SRG-15 mice inhibit tumor growth following treatment with rituximab (RTX). FIG. 25B, shows the frequency of human NK cells and T cells in human tumor xenografts of untreated (n=5) and RTX-treated SRG-15 mice (n=1). FIG. 25C, shows human NK cell subsets in the blood and tumor of untreated (n=2) and RTX-treated SRG-15 mice (n=1).

### Example 10: Additional Materials and Methods Utilized in Connection with the Above Examples

Human CD34⁺ cell isolation and injection. Human CD34⁺ cell isolation and injection was performed according to the methods described, for example, in Rongvaux A, Willinger T, Martinek J et al. Nat Biotechnol 2014; 32:364-372.

Flow cytometric analysis of human cell populations. Flow cytometric analysis of human cell populations was performed as described in Strowig T, Rongvaux A, Rathinam C et al. Proc Natl Acad Sci USA 2011; 108:13218-13223, and in Rongvaux A, Willinger T, Martinek J et al. Nat Biotechnol 2014; 32:364-372.

Histology. Tissue was fixed overnight in 4% paraformaldehyde, transferred to 70% ethanol and embedded in paraffin.

Quantitative RT-PCR. Quantitative RT-PCR was performed as described in Rongvaux A, Willinger T, Martinek J et al. Nat Biotechnol 2014; 32:364-372.

16S rRNA sequencing. 16S rRNA sequencing was performed as described in Palm N W, de Zoete M R, Cullen T W et al. Cell 2014; 158:1000-1010.

Viral infections. Rotavirus and influenza virus were obtained and applied in the subject methods.

Statistical analysis. Statistical significance was performed with Prism 6 software (GraphPad), using two-tailed unpaired Student's t-test.

FACS antibodies were obtained BD Biosciences and BioLegend.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

ADDITIONAL SEQUENCE INFORMATION

```
LOCUS NM_001040022 4201 bp mRNA linear PRI 15 Mar. 2015
DEFINITION Homo sapiens signal-regulatory protein alpha (SIRPA),
                        transcript variant 1, mRNA.
ACCESSION NM_001040022
VERSION NM_001040022.1 GI: 91105763
SOURCE Homo sapiens (human)
[SEQ ID NO: 11]
      1 tccggcccgc acccaccccc aagaggggcc ttcagctttg gggctcagag gcacgacctc
    61 ctggggaggg ttaaaaggca gacgcccccc cgccccccgc gcccccgcgc cccgactcct
  121 tcgccgcctc cagcctctcg ccagtgggaa gcggggagca gccgcgcggc cggagtccgg
  181 aggcgagggg aggtcggccg caacttcccc ggtccacctt aagaggacga tgtagccagc
  241 tcgcagcgct gaccttagaa aaacaagttt gcgcaaagtg gagcggggac ccggcctctg
  301 ggcagccccg gcggcgcttc cagtgccttc cagccctcgc gggcggcgca gccgcggccc
  361 atggagcccg ccggcccggc ccccggccgc ctcgggccgc tgctctgcct gctgctcgcc
  421 gcgtcctgcg cctggtcagg agtggcgggt gaggaggagc tgcaggtgat tcagcctgac
  481 aagtccgtgt tcgttgcagc tggagagaca gccactctgc gctgcactgc gacctctctg
  541 atccctgtgg ggcccatcca gtggttcaga ggagctggac caggccggga attaatctac
  601 aatcaaaaag aaggccactt cccccgggta acaactgttt cagacctcac aaagagaaac
  661 aacatggact tttccatccg catcggtaac atcaccccag cagatgccgg cacctactac
  721 tgtgtgaagt tccggaaagg gagccccgat gacgtggagt ttaagtctgg agcaggcact
  781 gagctgtctg tgcgcgccaa accctctgcc cccgtggtat cgggccctgc ggcgagggcc
  841 acacctcagc acacagtgag cttcacctgc gagtcccacg gcttctcacc cagagacatc
  901 accctgaaat gcttcaaaaa tgggaatgag ctctcagact tccagaccaa cgtggacccc
  961 gtaggagaga gcgtgtccta cagcatccac agcacagcca aggtggtgct gacccgcgag
1021 gacgttcact ctcaagtcat ctgcgaggtg gcccacgtca ccttgcaggg ggaccctctt
1081 cgtgggactg ccaacttgtc tgagaccatc cgagttccac ccaccttgga ggttactcaa
1141 cagcccgtga gggcagagaa ccaggtgaat gtcacctgcc aggtgaggaa gttctacccc
1201 cagagactac agctgacctg gttggagaat ggaaacgtgt cccggacaga aacggcctca
1261 accgttacag agaacaagga tcgtacctac aactggatga gctggctcct ggtgaatgta
1321 tctgcccaca gggatgatgt gaagctcacc tcccaggtgg agcatgacgg gcagccagcg
1381 gtcagcaaaa gccatgacct gaaggtctca gcccacccga aggagcaggg ctcaaatacc
1441 gccgctgaga acactggatc taatgaacgg aacatctata ttgtggtggg tgtggtgtgc
1501 accttgctgg tcgccctact gatggcggcc ctctacctcg tccgaatcag acagaagaaa
1561 gcccagggct ccacttcttc tacaaggttg catgagcccg agaagaatgc cagagaaata
1621 acacaggaca caaatgatat cacatatgca gacctgaacc tgcccaaggg gaagaagcct
1681 gctccccagg ctgcggagcc caacaaccac acggagtatg ccagcattca gaccagcccg
1741 cagcccgcgt cggaggacac cctcacctat gctgacctgg acatggtcca cctcaaccgg
1801 acccccaagc agccggcccc caagcctgag ccgtccttct cagagtacgc cagcgtccag
1861 gtcccgagga agtgaatggg accgtggttt gctctagcac ccatctctac gcgctttctt
1921 gtcccacagg gagccgccgt gatgagcaca gccaacccag ttcccggagg gctggggcgg
1981 tccaggctct gggacccagg ggccagggtg gctcttctct ccccacccct ccttggctct
2041 ccagcacttc ctgggcagcc acggccccct ccccccacat tcccacatac ctggaggctg
2101 acgttcccaa accagccagg gaaccaacct gggaagtggc cagaactgcc tggggtccaa
2161 gaactcttgt gcctccgtcc atcaccatgt gggttttgaa gaccctcgac tgcctccccg
2221 atgctccgaa gcctgatctt ccagggtggg gaggagaaaa tcccacctcc cctgacctcc
2281 accacctcca ccaccaccac caccaccacc accaccacta ccaccaccac ccaactgggg
2341 ctagagtggg gaagatttcc cctttagatc aaactgcccc ttccatggaa aagctggaaa
2401 aaaactctgg aacccatatc caggcttggt gaggttgctg ccaacagtcc tggcctcccc
2461 catccctagg ctaaagagcc atgagtcctg gaggaggaga ggacccctcc caaaggactg
2521 gagacaaaac cctctgcttc cttgggtccc tccaagactc cctggggccc aactgtgttg
2581 ctccacccgg acccatctct cccttctaga cctgagcttg cccctccagc tagcactaag
2641 caacatctcg ctgtggacgc ctgtaaatta ctgagaaatg tgaaacgtgc aatcttgaaa
2701 ctgaggtgtt agaaaacttg atctgtggtg ttttgttttg ttttttttct taaaacaaca
2761 gcaacgtgat cttggctgtc tgtcatgtgt tgaagtccat ggttgggtct tgtgaagtct
2821 gaggtttaac agtttgttgt cctggaggga ttttcttaca gcgaagactt gagttcctcc
2881 aagtcccaga accccaagaa tgggcaagaa ggatcaggtc agccactccc tggagacaca
2941 gccttctggc tcggactgac ttggccatgt tctcagctga gccacgcggc tcgtagtgca
3001 gccttctgtg accccgctgt ggtaagtcca gcctgcccag ggctgctgag ggctgcctct
3061 tgacagtgca gtcttatcga gacccaatgc ctcagtctgc tcatccgtaa agtggggata
3121 gtgaagatga cacccctccc caccacctct cataagcact ttaggaacac acagaggcta
3181 gggatagtgg ccctggccgt ctatcctacc cctttagtga ccgcccccat cccggctttc
3241 tgagctgatc cttgaagaag aaatcttcca tttctgctct caaaccctac tcggatcaaa
3301 ctggaataaa ttgaagacag ccagggggat ggtgcagctg tgaagctcgg gctgattccc
3361 cctctgtccc agaaggttgg ccagagggtg tgacccagtt accctttaac ccccaccctt
3421 ccagtcgggt gtgagggcct gaccgggccc agggcaagca gatgtcgcaa gccctattta
3481 ttcagtcttc actataactc ttagagttga gacgctaatg ttcatgactc ctggccttgg
3541 gatgcccaag ggatttctgg ctcaggctgt aaaagtagct gagccatcct gcccattcct
3601 ggaggtccta caggtgaaac tccaggagct cagcatagac ccagctctct gggggatggt
3661 cacctggtga tttcaatgat ggcatccagg aattagctga gccaacagac catgtggaca
3721 gctttggcca gagctcccgt gtggcatctg ggagccacag tgacccagcc acctggctca
3781 ggctagttcc aaattccaaa agattggctt gtaaaccttc gtctccctct cttttaccca
3841 gagacagcac atacgtgtgc acacgcatgc acacacacat tcagtatttt aaaagaatgt
3901 tttcttggtg ccattttcat tttattttat tttttaattc ttggaggggg aaataaggga
3961 ataaggccaa ggaagatgta tagctttagc tttagcctgg caacctggag aatccacata
4021 ccttgtgtat tgaaccccag gaaaaggaag aggtcgaacc aaccctgcgg aaggagcatg
4081 gtttcaggag tttattttaa gactgctggg aaggaaacag gccccatttt gtatatagtt
4141 gcaacttaaa ctttttggct tgcaaaatat ttttgtaata aagatttctg ggtaataatg
4201 a
[SEQ ID NO: 12]
Translation = MEPAGPAPGRLGPLLCLLLAASCAWSGVAGEEELQVIQPDKSVLVAAGETATLRCT
ATSLIPVGPIQWFRGAGPGRELIYNQKEGHFPRVTTVSDLTKRNNMDFISRIGNITPADAGTYYCVKFRK
GSPDDVEFKSGAGTELSVRAKPSAPVVSGPAARATPQHTVSFTCESHGFSPRDITLKWFKNGNELSDFQT
NVDPVGESVSYSIHSTAKVVLTREDVHSQVICEVAHVTLQGDPLRGTANLSETIRVPPTLVEVTQQPVRA
ENQVNTCQVRKFYPQRLQLTWLENGNVSRTETASTVTENKDGTYNWMSWLLVNVSAHRDDVKLTCQVEHD
GQPAVSKSHDLKVSAHPKEQGSNTAAENTGSNERNIYIVVGVVCTLLVALLMAALYLVRIRQKKAQGSTS
STRLHEPEKNAREITQDTNDITYADLNLPKGKKPAPQAAEPNNHTEYASIQTSPQPASEDTLTYADLDMV
HLNRTPKQPAPKPEPSFSEYASVQVPRK
LOCUS NM_001040023 4109 bp mRNA linear PRI 15 Mar. 2015
DEFINITION Homo sapiens signal-regulatory protein alpha (SIRPA),
                        Transcript variant 2, mRNA.
ACCESSION NM_001040023
VERSION NM_001040023.1 GI: 91105766
SOURCE Homo sapiens (human)
[SEQ ID NO: 13]
      1 ctctctggcc gcccctggct ttatttctcg cgcgcttggg gtctctccca gtctccgtct
    61 ctccatttct cctggggggc ggggaggggg ggtctccaaa aaccgcggcg gcggcggcgg
  121 ccgctccagg cgcccgttcc ggagtcgggg ggaggcccag ccgggagggg ggaagggggg
  181 gagccttagt catttccccg ctccagcctg ctcccgcccg agcgcgcact cacggccgct
  241 ctccctcctc gctccgcagc cgcggcccat ggagcccgcc ggcccggccc ccggccgcct
  301 cgggccgctg ctctgcctgc tgctcgccgc gtcctgcgcc tggtcaggag tggcgggtga
  361 ggaggagctg caggtgattc agcctgacaa gtccgtgttg gttgcagctg gagagacagc
  421 cactctgcgc tccactgcga cctctctgat ccctgtgggg cccatccagt ggttcagagg
  481 agctggacca ggccgggaat taatctacaa tcaaaaagaa ggccacttcc cccgggtaac
  541 aactctttca gacctcacaa agagaaacaa catggacttt tccatccgca tcggtaacat
  601 caccccagca gatgccggca cctactactg tgtgaagttc cggaaaggga gccccgatga
  661 cgtggagttt aagtctggag caggcactga gctgtctgtg cgcgccaaac cctctgcccc
  721 cgtcgtatcg ggccctgcgg cgagggccac acctcagcac acagtgagct tcacctgcga
  781 gtcccacggc ttctcaccca gagacatcac cctgaaatgg ttcaaaaatg ggaatgagct
  841 ctcagacttc cagaccaacg tggaccccgt aggagagagc gtgtcctaca gcatccacag
  901 cacagccaag gtggtgctga cccgcgagga cgttcactct caagtcatct gcgaggtggc
  961 ccacgtcacc ttgcaggggg accctcttcg tgggactgcc aacttgtctg agaccatccg
1021 agttccaccc accttggagg ttactcaaca gcccgtgagg gcagagaacc aggtgaatgt
1081 cacctgccag gtgaggaagt tctaccccca gagactacag ctgacctggt tggagaatgg
1141 aaacgtgtcc cggacagaaa cggcctcaac cgttacagag aacaaggatg gtacctacaa
1201 ctggatgagc tcgctcctgg tgaatgtatc tgcccacagg gatgatgtga agctcacctg
1261 ccaggtggag catgacgggc agccagcggt cagcaaaagc catgacctga aggtctcagc
1321 ccacccgaag gagcagggct caaataccgc cgctgagaac actggatcta atgaacggaa
1381 catctatatt gtggtgggtg tggtgtgcac cttgctggtg gccctactga tggcggccct
1441 ctacctcgtc cgaatcagac agaagaaagc ccagggctcc acttcttcta caaggttgca
1501 tgagcccgag aagaatgcca gagaaataac acaggacaca aatgatatca catatgcaga
1561 cctgaacctg cccaagggga agaagcctgc tccccaggct gcggagccca acaaccacac
1621 ggagtatgcc agcattcaga ccagcccgca gcccgcgtcg gaggacaccc tcacctatgc
1681 tgacctggac atggtccacc tcaaccggac ccccaagcag ccggccccca agcctgagcc
1741 gtccttctca gagtacgcca gcgtccaggt cccgaggaag tgaatgggac cgtggtttgc
1801 tctagcaccc atctctacgc gctttcttgt cccacaggga gccgccgtga tgagcacagc
1861 caacccagtt cccggagggc tggggcggtg caggctctgg gacccagggg ccagggtggc
1921 tcttctctcc ccacccctcc ttggctctcc agcacttcct gggcagccac ggccccctcc
1981 ccccacattg ccacatacct ggaggctgac gttgccaaac cagccaggga accaacctgg
2041 gaagtggcca gaactgcctg gggtccaaga actcttgtgc ctccgtccat caccatgtgg
2101 gttttgaaga ccctcgactg cctccccgat gctccgaagc ctgatcttcc agggtgggga
2161 ggagaaaatc ccacctcccc tgacctccac cacctccacc accaccacca ccaccaccac
2221 caccactacc accaccaccc aactggggct agagtgggga agatttcccc tttagatcaa
2281 actgcccctt ccatggaaaa gctggaaaaa aactctggaa cccatatcca ggcttggtga
2341 ggttgctgcc aacagtcctg gcctccccca tccctaggct aaagagccat gagtcctgga
2401 ggaggagagg acccctccca aaggactgga gacaaaaccc tctgcttcct tgggtccctc
2461 caagactccc tggggcccaa ctgtgttgct ccacccggac ccatctctcc cttctagacc
2521 tgagcttgcc cctccagcta gcactaagca acatctcgct gtggacgcct gtaaattact
2581 gagaaatgtg aaacgtgcaa tcttgaaact gaggtgttag aaaacttgat ctgtggtgtt
2641 ttgttttgtt ttttttctta aaacaacagc aacgtgatct tggctgtctg tcatgtgttg
2701 aagtccatgg ttgggtcttg tgaagtctga ggtttaacag tttgttgtcc tggagggatt
2761 ttcttacagc gaagacttga gttcctccaa gtcccagaac cccaagaatg ggcaagaagg
2821 atcaggtcag ccactccctg gagacacagc cttctggctg ggactgactt ggccatgttc
2881 tcagctgagc cacgcggctg gtagtgcagc cttctgtgac cccgctgtgg taagtccagc
2941 ctgcccaggg ctgctgaggg ctgcctcttg acagtgcagt cttatcgaga cccaatgcct
3001 cagtctgctc atccgtaaag tggggatagt gaagatgaca cccctcccca ccacctctca
3061 taagcacttt aggaacacac agagggtagg gatagtggcc ctggccgtct atcctacccc
3121 tttagtgacc gcccccatcc cggctttctg agctgatcct tgaagaagaa atcttccatt
3181 tctgctctca aaccctactg ggatcaaact ggaataaatt gaagacagcc agggggatgg
3241 tgcagctgtg aagctcgggc tgattccccc tctgtcccag aaggttggcc agagggtgtg
3301 acccagttac cctttaaccc ccacccttcc agtcgggtgt gagggcctga ccgggcccag
3361 ggcaagcaga tgtcgcaagc cctatttatt cagtcttcac tataactctt agagttgaga
3421 cgctaatgtt catgactcct ggccttggga tgcccaaggg atttctggct caggctgtaa
3481 aagtagctga gccatcctgc ccattcctgg aggtcctaca ggtgaaactg caggagctca
3541 gcatagaccc agctctctgg gggatggtca cctggtgatt tcaatgatgg catccaggaa
3601 ttagctgagc caacagacca tgtggacagc tttggccaga gctcccgtgt ggcatctggg
3661 agccacagtg acccagccac ctggctcagg ctagttccaa attccaaaag attggcttgt
3721 aaaccttcgt ctccctctct tttacccaga gacagcacat acgtgtgcac acgcatgcac
3781 acacacattc agtattttaa aagaatgttt tcttggtgcc attttcattt tattttattt
3841 tttaattctt ggagggggaa ataagggaat aaggccaagg aagatgtata gctttagctt
3901 tagcctggca acctggagaa tccacatacc ttgtgtattg aaccccagga aaaggaagag
3961 gtcgaaccaa ccctgcggaa ggagcatggt ttcaggagtt tattttaaga ctgctgggaa
4021 ggaaacaggc cccattttgt atatagttgc aacttaaact ttttggcttg caaaatattt
4081 ttgtaataaa gatttctggg taataatga
[SEQ ID NO: 12]
Translation = MEPAGPAPGRLGPLLCLLLAASCAWSGVAGEEELQVIQPDKSVLVAAGETATLRCT
ATSLIPVGPIQWFRGAGPGRELIYNQKEGHFPRVTTVSDLTKRNNMDESIRIGNITPADAGTYYCVKFRK
GSPDDVEFKSGAGTELSVRAKPSAPVVSGPAARATPQHTVSFTCESHGFSPRDITLKWFKNGNELSDFQT
NVDPVGESVSYSIHSTAKVVLTREDVHSQVICEVAHVTLQGDPLRGTANLSETIRVPPTLEVTQQPVRAE
NQVNVTCQVRKFYPQRLQLTWLENGNVSRTETASTVTENKDGTYNWMSWLLVNVSAHRDDVKLTCQVEHD
GQPAVSKSHDLKVSAHPKEQGSNTAAENTGSNERNIYIVVGVVCTLLVALLMAALYLVRIRQKKAQGSTS
STRLHEPEKNAREITQDTNDITYADLNLPKGKKPAPQAAEPNNHTEYASIQTSPQPASEDTLTYADLDMV
HLNRTPKQPAPKPEPSFSEYASVQVPRK
LOCUS NM_080792 3868 bp mRNA linear PRI 15 Mar. 2015
DEFINITION Homo sapiens signal-regulatory protein alpha (SIRPA),
                        Transcript variant 3, mRNA.
ACCESSION NM_080792 NM_004648
VERSION NM_080792.2 GI: 91105786
SOURCE Homo sapiens (human)
[SEQ ID NO: 14]
      1 cgctcgctcg cagagaagcc gcggcccatg gagcccgccg gcccggcccc cggccgcctc
    61 gggccgctgc tctgcctgct gctcgccgcg tcctgcgcct ggtcaggagt ggcgggtgag
  121 gaggagctgc aggtgattca gcctgacaag tccgtgttgg ttgcagctgg agagacagcc
  181 actctgcgct gcactgcgac ctctctgatc cctgtggggc ccatccagtg gttcagagga
  241 gctggaccag gccgggaatt aatctacaat caaaaagaag gccacttccc ccgggtaaca
  301 actgtttcag acctcacaaa gagaaacaac atggactttt ccatccgcat cggtaacatc
  361 accccagcag atgccggcac ctactactgt gtgaagttcc ggaaagggag ccccgatgac
  421 gtggagttta agtctggagc aggcactgag ctgtctgtgc gcgccaaacc ctctgccccc
  481 gtggtatcgg gccctgcggc gagggccaca cctcagcaca cagtgagctt cacctgcgag
  541 tcccacggct tctcacccag agacatcacc ctgaaatggt tcaaaaatgg gaatgagctc
  601 tcagacttcc agaccaacgt ggaccccgta ggagagagcg tgtcctacag catccacagc
  661 acagccaagg tcgtgctgac ccgcgaggac gttcactctc aagtcatctg cgaggtggcc
  721 cacgtcacct tccaggggga ccctcttcgt gggactgcca acttgtctga gaccatccga
  781 gttccaccca ccttggaggt tactcaacag cccgtgaggg cagagaacca ggtgaatgtc
  841 acctgccagg tgaggaagtt ctacccccag agactacagc tgacctggtt ggagaatgga
  901 aacgtgtccc ggacagaaac ggcctcaacc gttacagaga acaaggatgg tacctacaac
  961 tcgatgagct ggctcctggt gaatgtatct gcccacaggg atgatgtgaa gctcacctgc
1021 caggtggagc atgacgggca gccagcggtc agcaaaagcc atgacctgaa ggtctcagcc
1081 cacccgaagg agcagggctc aaataccgcc gctgagaaca ctggatctaa tgaacggaac
1141 atctatattg tggtgggtgt ggtgtgcacc ttgctggtgg ccctactgat ggcggccctc
1201 tacctcgtcc gaatcagaca gaagaaagcc cagggctcca cttcttctac aaggttgcat
1261 gagcccgaga agaatgccag agaaataaca caggacacaa atgatatcac atatgcagac
1321 ctgaacctgc ccaaggggaa gaagcctgct ccccaggctg cggagcccaa caaccacacg
1381 gagtatgcca gcattcagac cagcccgcag cccgcgtcgg aggacaccct cacctatgct
1441 gacctggaca tcgtccacct caaccggacc cccaagcagc cggcccccaa gcctgagccg
1501 tccttctcag agtacgccag cctccaggtc ccgaggaagt gaatgggacc gtggtttgct
1561 ctagcaccca tctctacgcg ctttcttgtc ccacagggag ccgccgtgat gagcacagcc
1621 aacccagttc ccggagggct ggggcggtgc aggctctggg acccaggggc cagggtggct
1681 cttctctccc cacccctcct tggctctcca gcacttcctg ggcagccacg gccccctccc
1741 cccacattgc cacatacctg gaggctgacg ttgccaaacc agccagggaa ccaacctggg
1801 aagtggccag aactgcctgg ggtccaagaa ctcttgtgcc tccgtccatc accatgtggg
1861 ttttgaagac cctcgactgc ctccccgatg ctccgaagcc tgatcttcca gggtggggag
1921 gagaaaatcc cacctcccct gacctccacc acctccacca ccaccaccac caccaccacc
1981 accactacca ccaccaccca actggggcta gagtggggaa gatttcccct ttagatcaaa
2041 ctgccccttc catggaaaag ctggaaaaaa actctggaac ccatatccag gcttggtgag
2101 gttgctgcca acagtcctgg cctcccccat ccctaggcta aagagccatg agtcctggag
2161 gaggagagga cccctcccaa aggactggag acaaaaccct ctgcttcctt gggtccctcc
2221 aagactccct ggggcccaac tgtgttgctc cacccggacc catctctccc ttctagacct
2281 gagcttgccc ctccagctag cactaagcaa catctcgctg tggacgcctg taaattactg
2341 agaaatgtga aacgtgcaat cttgaaactg aggtgttaga aaacttgatc tgtggtgttt
2401 tgttttgttt tttttcttaa aacaacagca acgtgatctt ggctgtctgt catgtgttga
2461 agtccatggt tcggtcttgt gaagtctgag gtttaacagt ttgttgtcct ggagggattt
2521 tcttacagcg aagacttgag ttcctccaag tcccagaacc ccaagaatgg gcaagaagga
2581 tcaggtcagc cactccctgg agacacagcc ttctggctgg gactgacttg gccatgttct
2641 cagctgagcc acgcggctgg tagtgcagcc ttctgtgacc ccgctgtggt aagtccagcc
2701 tgcccagggc tgctgagggc tgcctcttga cagtgcagtc ttatcgagac ccaatgcctc
2761 agtctgctca tccgtaaagt ggggatagtg aagatgacac ccctccccac cacctctcat
2821 aagcacttta ggaacacaca gagggtaggg atagtggccc tggccgtcta tcctacccct
2881 ttagtgaccg cccccatccc ggctttctga gctgatcctt gaagaagaaa tcttccattt
2941 ctgctctcaa accctactgg gatcaaactg gaataaattg aagacagcca gggggatggt
3001 gcagctgtga agctcgggct gattccccct ctgtcccaga aggttggcca gagggtgtga
3061 cccagttacc ctttaacccc cacccttcca gtcgggtgtg agggcctgac cgggcccagg
3121 gcaagcagat gtcgcaagcc ctatttattc agtcttcact ataactctta gagttgagac
3181 gctaatgttc atgactcctg gccttgggat gcccaaggga tttctggctc aggctgtaaa
3241 agtagctgag ccatcctgcc cattcctgga ggtcctacag gtgaaactgc aggagctcag
3301 catagaccca gctctctggg ggatggtcac ctggtgattt caatgatggc atccaggaat
3361 tagctgagcs aacagaccat gtggacagct ttggccagag ctcccgtgtg gcatctggga
3421 gccacagtga cccagccacc tggctcaggc tagttccaaa ttccaaaaga ttggcttgta
3481 aaccttcgtc tccctctctt ttacccagag acagcacata cgtgtgcaca cgcatgcaca
3541 cacacattca gtattttaaa agaatgtttt cttggtgcca ttttcatttt attttatttt
3601 ttaattcttg gagggggaaa taagggaata aggccaagga agatgtatag ctttagcttt
3661 agcctggcaa cctggagaat ccacatacct tctgtattga accccaggaa aaggaagagg
3721 tcgaaccaac cctgcggaag gagcatggtt tcaggagttt attttaagac tgctgggaag
3781 gaaacaggcc ccattttgta tatagttgca acttaaactt tttggcttgc aaaatatttt
3841 tctaataaag atttctgggt aataatga
[SEQ ID NO: 12]
Translation = MEPAGPAPGRLGPLLCLLLAASCAWSGVAGEEELQVIQPDKSVLVAAGETATLRCT
ATSLIPVGPIQWFRGAGPGRELIYNQKEGHFPRVTTVSDLTKRNNMDESIRIGNITPADAGTYYCVKFRK
GSPDDVEFKSGAGTELSVRAKPSAPVVSGPAARATPQHTVSFTCESHGFSPRDITLKWFKNGNELSDFQT
NVDPVGESVSYSIHSTAKVVLTREDVHSQVICEVAHVTLQGDPLRGTANLSETIRVPPTLEVTQQPVRAE
NQVNVTCQVRKFYPQRLQLTWLENGNVSRTETASTVTENKDGTYNWMSWLLVNVSAHRDDVKLTCQVEHD
GQPAVSKSHDLKVSAHPKEQGSNTAAENTGSNERNIYIVVGVVCTLLVALLMAALYLVRIRQKKAQGSTS
STRLHEPEKNAREITQDTNDITYADLNLPKGKKPAPQAAEPNNHTEYASIQTSPQPASEDTLTYADLDMV
HLNRTPKQPAPKPEPSFSEYASVQVPRK
LOCUS NM_007547 4031 bp mRNA linear ROD 15 Feb. 2015
DEFINITION Mus musculus signal-regulatory protein alpha (Sirpa),
                        Transcript variant 1, mRNA.
ACCESSION NM_007547 NM_011208
VERSION NM_007547.4 GI: 597084939
SOURCE Mus musculus (house mouse)
[SEQ ID NO: 15]
      1 cgggaaggtg cgggcgcgag gagggggcgc tcggccgggc cgccctcgcg ctggcctcgc
    61 gacggctccg cacagcccgc actcgctctg cgagctgtcc ccgctcgcgc ttgctctccg
  121 atctccgtcc ccgctccctc tccctcttcc tctccccctc tttccttctc cctcgctatc
  181 cgctcccccg cccccgtgcc tctggctctg cgcctggctc cctcgggtcc gctccccttt
  241 cccgccggcs tggcccggcg tcacgctccc ggagtctccc cgctcggcgg cgtctcattg
  301 tcggaggggg tcagatcacc ccgccgggcg gtggcgctgg ggggcagcgg agggggaggg
  361 gccttagtcg ttcgcccgcg ccgcccgccc gcctgccgag cgcgctcacc gccgctctcc
  421 ctccttgctc tccagccgcg gcccatggag cccgccggcc cggcccctgg ccgcctaggg
  481 ccgctgctgc tctgcctgct gctctccgcg tcctgtttct gtacaggagc cacggggaag
  541 gaactgaagg tgactcagcc tgagaaatca gtgtctgttg ctgctgggga ttcgaccgtt
  601 ctgaactgca ctttgacctc cttgttgccg gtgggaccca ttaggtggta cagaggagta
  661 gggccaagcc ggctgttgat ctacagtttc gcaggagaat acgttcctcg aattagaaat
  721 gtttcagata ctactaagag aaacaatatg gacttttcca tccgtatcag taatgtcacc
  781 ccagcagatg ctggcatcta ctactgtgtg aagttccaga aaggatcatc agagcctgac
  841 acagaaatac aatctggagg gggaacagag gtctatgtac tcgccaaacc ttctccaccg
  901 gaggtatccg gcccagcaga caggggcata cctgaccaga aagtgaactt cacctgcaag
  961 tctcatggct tctctccccg gaatatcacc ctgaagtggt tcaaagatgg gcaagaactc
1021 caccccttgg agaccaccgt gaaccctagt ggaaagaatg tctcctacaa catctccagc
1081 acagtcaggg tcgtactaaa ctccatggat gttaattcta aggtcatctg cgaggtagcc
1141 cacatcacct tcgatagaag ccctcttcgt gggattgcta acctgtctaa cttcatccga
1201 gtttcaccca ccgtgaaggt cacccaacag tccccgacgt caatgaacca ggtgaacctc
1261 acctgccggg ctgagaggtt ctaccccgag gatctccagc tgatctggct ggagaatgga
1321 aacgtatcac ggaatgacac gcccaagaat ctcacaaaga acacggatgg gacctataat
1381 tacacaagct tcttcctggt gaactcatct gctcatagag aggacgtggt gttcacgtgc
1441 caggtgaagc acgaccaaca gccagcgatc acccgaaacc ataccgtgct gggatttgcc
1501 cactcgagtg atcaagggag catgcaaacc ttccctgata ataatgctac ccacaactgg
1561 aatgtcttca tcggtgtggg cgtggcgtgt gctttgctcg tagtcctgct gatggctgct
1621 ctctacctcc tccggatcaa acagaagaaa gccaaggggt caacatcttc cacacggttg
1681 cacgagcccg agaagaacgc cagggaaata acccagatcc aggacacaaa tgacatcaac
1741 gacatcacat acgcagacct gaatctgccc aaagagaaga agcccgcacc ccgggcccct
1801 gagcctaaca accacacaga atatgcaagc attgagacag gcaaagtgcc taggccagag
1861 gataccctca cctatgctga cctggacatg gtccacctca gccgggcaca gccagccccc
1921 aagcctgagc catctttctc agagtatgct agtgtccagg tccagaggaa gtgaatgggg
1981 ctgtggtctg tactaggccc catccccaca agttttcttg tcctacatgg agtggccatg
2041 acgaggacat ccagccagcc aatcctgtcc ccagaaggcc aggtggcacg ggtcctagga
2101 ccaggggtaa gggtggcctt tgtcttccct ccgtggctct tcaacacctc ttgggcaccc
2161 acgtcccctt cttccggagg ctgggtgttg cagaaccaga gggcgaactg gagaaagctg
2221 cctggaatcc aagaagtgtt gtgcctcggc ccatcactcg tcggtctgga tcctggtctt
2281 ggcaacccca ggttgcgtcc ttgatgttcc agagcttggt cttctgtgtg gagaagagct
2341 caccatctct acccaacttg agctttggga ccagactccc tttagatcaa accgccccat
2401 ctgtggaaga actacaccag aagtcagcaa gttttcagcc aacagtgctg gcctccccac
2461 ctcccaggct gactagccct ggggagaagg aaccctctcc tcctagacca gcagagactc
2521 cctgggcatg ttcagtgtgg ccccacctcc cttccagtcc cagcttgctt cctccagcta
2581 gcactaactc agcagcatcg ctctgtggac gcctgtaaat tattgagaaa tgtgaactgt
2641 gcagtcttaa agctaaggtg ttagaaaatt tgatttatgc tctttagttg ttgttgggtt
2701 tcttttcttt ttaatttctt tttctttttt gatttttttt ctttccctta aaacaacagc
2761 agcagcatct tcgctctttg tcatgtgttg aatggttggg tcttgtgaag tctgaggtct
2821 aacagtttat tgtcctggaa ggattttctt acagcagaaa cagatttttt tcaaattccc
2881 agaatcctga ggaccaagaa ggatccctca gctgctactt ccagcaccca gcgtcactgg
2941 gacgaaccag gccctgttct tacaaggcca catggctggc cctttgcctc catggctact
3001 gtcgtaagtg cagccttgtc tgacccaatg ctgacctaat gttggccatt ccacattgag
3061 gggacaaggt cagtgatgcc ccccttcact cacaagcact tcagaggcat gcagagagaa
3121 gggacactcg gccagctctc tgaggtaatc agtgcaagga ggagtccgtt ttttgccagc
3181 aaacctcagc aggatcacac tggaacagaa cctggtcata cctgtgacaa cacagctgtg
3241 agccagggca aaccacccac tgtcactggc tcgagagtct gggcagaggc tctgaccctc
3301 caccctttaa actggatgcc ggggcctggc tcggcccaat gccaagtggt tatggcaacc
3361 ctgactatct ggtcttaaca tgtagctcag gaagtggagg cgctaatgtc cccaatccct
3421 ggggattcct gattccagct attcatgtaa gcagagccaa cctgcctatt tctgtaggtg
3481 cgactcggat gttaggagca cagcaaggac ccagctctgt agggctggtg acctgatact
3541 tctcataatg gcatctagaa gttaggctga gttggcctca ctggcccagc aaaccagaac
3601 ttgtctttgt ccgggccatg ttcttgggct gtcttctaat tccaaagggt tggttggtaa
3661 agctccaccc ccttctcctc tgcctaaaga catcacatgt gtatacacac acgggtgtat
3721 agatgagtta aaagaatgtc ctcgctggca tcctaatttt gtcttaagtt tttttggagg
3781 gagaaaggaa caaggcaagg gaagatgtgt agctttggct ttaaccaggc agcctggggg
3841 ctcccaagcs tatggaaccc tggtacaaag aagagaacag aagcgccctg tgaggagtgg
3901 gatttgtttt tctgtagacc agatgagaag gaaacaggcc ctgttttgta catagttgca
3961 acttaaaatt tttggcttgc aaaatatttt tgtaataaag atttctgggt aacaataaaa
4021 aaaaaaaaaa a
[SEQ ID NO: 16]
Translation = MEPAGPAPGRLGPLLLCLLLSASCFCTGATGKELKVTQPEKSVSVAAGDSTVLNCT
LTSLLPVGPIRWYRGVGPSRLLIYSFAGEYVPRIRNVSDTTKRNNMDFSIRISNVTPADAGIYYCVKFQK
GSSEPDTEIQSGGGTEVYVLAKPSPPEVSGPADRGIPDQKVNFTCKSHGFSPRNITLKWFKDGQELHPLE
TTVNPSGKNVSYNISSTVRVVLNSMDVNSKVICEVAHITLDRSPLRGIANLSNFIRVSPTVKVTQQSPTS
MNQVNLTCRAERFYPEDLQLIWLENGNVSRNDTPKNLTKNTDGTYNYTSLFLVNSSAHREDVVFTCQVKH
DQQPAITRNHTVLGFAHSSDQGSMQTFPDNNATHNWNVFIGVGVACALLVVLLMAALYLLRIKQKKAKGS
TSSTRLHEPEKNAREITQIQDTNDINDITYADLNLPKEKKPAPRAPEPNNHTEYASIETGKVPRPEDTLT
YADLDMVHLSRAQPAPKPEPSFSEYASVQVQRK
LOCUS NM_001177647 3377 bp mRNA linear ROD 15 Feb. 2015
DEFINITION Mus musculus signal-regulatory protein alpha (Sirpa),
                        Transcript variant 3, mRNA.
ACCESSION NM_001177647
VERSION NM_001177647.2 GI: 597436949
SOURCE Mus musculus (house mouse)
[SEQ ID NO: 17]
      1 cgggaaggtg cgggcgcgag gagggggcgc tcggccgggc cgccctcgcg ctggcctcgc
    61 gacggctccg cacagcccgc actcgctctg cgagctgtcc ccgctcgcgc ttgctctccg
  121 atctccgtcc ccgctccctc tccctcttcc tctccccctc tttccttctc cctcgctatc
  181 cgctcccccg cccccgtgcc tctggctctg cgcctggctc cctcgggtcc gctccccttt
  241 cccgccggcc tggcccggcg tcacgctccc ggagtctccc cgctcggcgg cgtctcattg
  301 tgggaggggg tcagatcacc ccgccgggcg gtggcgctgg ggggcagcgg agggggaggg
  361 gccttagtcg ttcgcccgcg ccgcccgccc gcctgccgag cgcgctcacc gccgctctcc
  421 ctccttgctc tgcagccgcg gcccatggag cccgccggcc cggcccctgg ccgcctaggg
  481 ccgctgctgc tctgcctgct gctctccgcg tcctgtttct gtacaggagc cacggggaag
  541 gaactgaagg tgactcagcc tgagaaatca gtgtctgttg ctgctgggga ttcgaccgtt
  601 ctgaactgca ctttgacctc cttgttgccg gtgggaccca ttaggtggta cagaggagta
  661 gggccaagcc ggctgttgat ctacagtttc gcaggagaat acgttcctcg aattagaaat
  721 gtttcagata ctactaagag aaacaatatg gacttttcca tccgtatcag taatgtcacc
  781 ccagcagatg ctggcatcta ctactgtgtg aagttccaga aaggatcatc agagcctgac
  841 acagaaatac aatctggagg gggaacagag gtctatgtac tcgataataa tcctacccac
  901 aactggaatg tcttcatcgg tgtgggcgtg gcgtgtgctt tgctcgtagt cctgctgatg
  961 gctgctctct acctcctccg gatcaaacag aagaaagcca aggggtcaac atcttccaca
1021 cggttgcacg agcccgagaa gaacgccagg gaaataaccc agatccagga cacaaatgac
1081 atcaacgaca tcacatacgc agacctgaat ctgcccaaag agaagaagcc cgcaccccgg
1141 gcccctgagc ctaacaacca cacagaatat gcaagcattg agacaggcaa agtgcctagg
1201 ccagaggata ccctcaccta tgctgacctg gacatggtcc acctcagccg ggcacagcca
1261 gcccccaagc ctgagccatc tttctcagag tatgctagtg tccaggtcca gaggaagtga
1321 atggggctgt ggtctgtact aggccccatc cccacaagtt ttcttgtcct acatggagtg
1381 gccatgacga ggacatccag ccagccaatc ctgtccccag aaggccaggt ggcacgggtc
1441 ctaggaccag gggtaagggt ggcctttgtc ttccctccgt ggctcttcaa cacctcttgg
1501 gcacccacgt ccccttcttc cggaggctgg gtgttgcaga accagagggc gaactggaga
1561 aagctgcctg gaatccaaga agtgttgtgc ctcggcccat cactcgtggg tctggatcct
1621 ggtcttggca accccaggtt gcgtccttga tcttccagag cttggtcttc tgtgtggaga
1681 agagctcacc atctctaccc aacttgagct ttgggaccag actcccttta gatcaaaccg
1741 ccccatctgt ggaagaacta caccagaagt cagcaagttt tcagccaaca gtgctggcct
1801 ccccacctcc caggctgact agccctgggg agaaggaacc ctctcctcct agaccagcag
1861 agactccctg ggcatgttca gcgtggcccc acctcccttc cagtcccagc ttgcttcctc
1921 cagctagcac taactcagca gcatcgctct gtggacgcct gtaaattatt gagaaatgtg
1981 aactgtgcag tcttaaagct aaggtgttag aaaatttgat ttatgctgtt tagttgttgt
2041 tgggtttctt ttctttttaa tttctttttc ttttttgatt ttttttcttt cccttaaaac
2101 aacagcagca gcatcttggc tctttgtcat gtgttgaatg gttgggtctt gtgaagtctg
2161 aggtctaaca gtttattgtc ctggaaggat tttcttacag cagaaacaga tttttttcaa
2221 attcccagaa tcctgaggac caagaaggat ccctcagctg ctacttccag cacccagcgt
2281 cactgggacg aaccaggccc tgttcttaca aggccacatg gctggccctt tgcctccatg
2341 gctactgtgg taagtgcagc cttgtctgac ccaatgctga cctaatgttg gccattccac
2401 attgagggga caaggtcagt gatgcccccc ttcactcaca agcacttcag aggcatgcag
2461 agagaaggga cactcggcca gctctctgag gtaatcagtg caaggaggag tccgtttttt
2521 gccagcaaac ctcagcagga tcacactgga acagaacctg gtcatacctg tgacaacaca
2581 gctgtgagcc agggcaaacc acccactgtc actggctcga gagtctgggc agaggctctg
2641 accctccacc ctttaaactg gatgccgggg cctggctggg cccaatgcca agtggttatg
2701 gcaaccctga ctatctggtc ttaacatgta gctcaggaag tggaggcgct aatgtcccca
2761 atccctgggg attcctgatt ccagctattc atgtaagcag agccaacctg cctatttctg
2821 taggtgcgas tgggatgtta ggagcacagc aaggacccag ctctgtaggg ctggtgacct
2881 gatacttctc ataatggcat ctagaagtta ggctgagttg gcctcactgg cccagcaaac
2941 cagaacttgt ctttgtccgg gccatgttct tgggctgtct tctaattcca aagggttggt
3001 tcgtaaagct ccaccccctt ctcctctgcc taaagacatc acatgtgtat acacacacgg
3061 gtgtatagat gagttaaaag aatgtcctcg ctggcatcct aattttgtct taagtttttt
3121 tcgagggaga aaggaacaag gcaagggaag atgtgtagct ttggctttaa ccaggcagcc
3181 tgggggctcc caagcctatg gaaccctggt acaaagaaga gaacagaagc gccctgtgag
3241 gagtgggatt tctttttctg tagaccagat gagaaggaaa caggccctgt tttgtacata
3301 gttgcaactt aaaatttttg gcttgcaaaa tatttttgta ataaagattt ctgggtaaca
3361 ataaaaaaaa aaaaaaa
[SEQ ID NO: 18]
Translation = MEPAGPAPGRLGPLLLCLLLSASCFCTGATGKELKVTQPEKSVSVAAGDSTVLNCT
LTSLLPVGPIRWYRGVGPSRLLIYSFAGEYVPRIRNVSDTTKRNNMDESIRISNVTPADAGIYYCVKFQK
GSSEPDTETQSGGGTEVYVLDNNATHNWNVFIGVGVACALLVVLLMAALYLLRIKQKKAKGSTSSTRLHE
PEKNAREITQIQDTNDINDITYADLNLPKEKKPAPRAPEPNNHTEYASIETGKVPRPEDTLTYADLDMVH
LSRAQPAPKPEPSESEYASVQVQRK
LOCUS NM_001291019 4043 bp mRNA linear ROD 15 Feb. 2015
DEFINITION Mus musculus signal-regulatory protein alpha (Sirpa),
                        transcript variant 4, mRNA.
ACCESSION NM_001291019 XM_006498985
VERSION NM_001291019.1 GI: 597436868
SOURCE Mus musculus (house mouse)
[SEQ ID NO: 19]
      1 cgggaaggtg cgggcgcgag gagggggcgc tcggccgggc cgccctcgcg ctggcctcgc
    61 gacggctccg cacagcccgc actcgctctg cgagctgtcc ccgctcgcgc ttgctctccg
  121 atctccgtcc ccgctccctc tccctcttcc tctccccctc tttccttctc cctcgctatc
  181 cgctcccccg cccccgtgcc tctggctctg cgcctggctc cctcgggtcc gctccccttt
  241 cccgccggcc tcgcccggcg tcacgctccc ggagtctccc cgctcggcgg cgtctcattg
  301 tcggaggggg tcagatcacc ccgccgggcg gtggcgctgg ggggcagcgg agggggaggg
  361 gccttagtcg ttcgcccgcg ccgcccgccc gcctgccgag cgcgctcacc gccgctctcc
  421 ctccttgctc tgcagccgcg gcccatggag cccgccggcc cggcccctgg ccgcctaggg
  481 ccgctgctgc tctgcctgct gctctccgcg tcctgtttct gtacaggagc cacggggaag
  541 gaactgaagg tgactcagcc tgagaaatca gtgtctgttg ctgctgggga ttcgaccgtt
  601 ctgaactgca ctttgacctc cttgttgccg gtgggaccca ttaggtggta cagaggagta
  661 gggccaagcc ggctgttgat ctacagtttc gcaggagaat acgttcctcg aattagaaat
  721 gtttcagata ctactaagag aaacaatatg gacttttcca tccgtatcag taatgtcacc
  781 ccagcagatg ctggcatcta ctactgtgtg aagttccaga aaggatcatc agagcctgac
  841 acagaaatac aatctggagg gggaacagag gtctatgtac tcgccaaacc ttctccaccg
  901 gaggtatccg gcccagcaga caggggcata cctgaccaga aagtgaactt cacctgcaag
  961 tctcatggct tctctccccg gaatatcacc ctgaagtggt tcaaagatgg gcaagaactc
1021 caccccttgg agaccaccgt gaaccctagt ggaaagaatg tctcctacaa catctccagc
1081 acagtcaggg tcgtactaaa ctccatggat gttaattcta aggtcatctg cgaggtagcc
1141 cacatcacct tggatagaag ccctcttcgt gggattgcta acctgtctaa cttcatccga
1201 gtttcaccca ccgtgaaggt cacccaacag tccccgacgt caatgaacca ggtgaacctc
1261 acctgccggg ctgagaggtt ctaccccgag gatctccagc tgatctggct ggagaatgga
1321 aacgtatcac ggaatgacac gcccaagaat ctcacaaaga acacggatgg gacctataat
1381 tacacaagct tcttcctggt gaactcatct gctcatagag aggacgtggt gttcacgtgc
1441 caggtgaagc acgaccaaca gccagcgatc acccgaaacc ataccgtgct gggatttgcc
1501 cactcgagtg atcaagggag catgcaaacc ttccctgata ataatgctac ccacaactgg
1561 aatgtcttca tcggtgtggg cgtggcgtgt gctttgctcg tagtcctgct gatggctgct
1621 ctctacctcc tccggatcaa acagaagaaa gccaaggggt caacatcttc cacacggttg
1681 cacgagcccg agaagaacgc cagggaaata acccaggtac agtctttgat ccaggacaca
1741 aatgacatca acgacatcac atacgcagac ctgaatctgc ccaaagagaa gaagcccgca
1801 ccccgggccc ctgagcctaa caaccacaca gaatatgcaa gcattgagac aggcaaagtg
1861 cctaggccag aggataccct cacctatgct gacctggaca tggtccacct cagccgggca
1921 cagccagccc ccaagcctga gccatctttc tcagagtatg ctagtgtcca ggtccagagg
1981 aagtgaatgg ggctgtggtc tgtactaggc cccatcccca caagttttct tctcctacat
2041 ggagtggcca tgacgaggac atccagccag ccaatcctgt ccccagaagg ccaggtggca
2101 cgggtcctag gaccaggggt aagggtggcc tttgtcttcc ctccgtggct cttcaacacc
2161 tcttgggcac ccacgtcccc ttcttccgga ggctgggtgt tgcagaacca gagggcgaac
2221 tggagaaagc tgcctggaat ccaagaagtg ttgtgcctcg gcccatcact cgtgggtctg
2281 gatcctggtc ttcgcaaccc caggttgcgt ccttgatgtt ccagagcttg gtcttctgtg
2341 tggagaagag ctcaccatct ctacccaact tgagctttgg gaccagactc cctttagatc
2401 aaaccgcccc atctgtggaa gaactacacc agaagtcagc aagttttcag ccaacagtgc
2461 tcgcctcccc acctcccagg ctgactagcc ctggggagaa ggaaccctct cctcctagac
2521 cagcagagac tccctgggca tgttcagtgt ggccccacct cccttccagt cccagcttgc
2581 ttcctccagc tagcactaac tcagcagcat cgctctgtgg acgcctgtaa attattgaga
2641 aatgtgaact gtgcagtctt aaagctaagg tcttagaaaa tttgatttat gctgtttagt
2701 tcttgttggg tttcttttct tcttaatttc tttttctttt ttgatttttt ttctttccct
2761 taaaacaaca gcagcagcat cttggctctt tgtcatgtgt tgaatggttg ggtcttgtga
2821 agtctgaggt ctaacagttt attgtcctgg aaggattttc ttacagcaga aacagatttt
2881 tttcaaattc ccagaatcct gaggaccaag aaggatccct cagctgctac ttccagcacc
2941 cagcgtcact gggacgaacc aggccctgtt cttacaaggc cacatggctg gccctttgcc
3001 tccatggcta ctgtggtaag tgcagccttg tctgacccaa tgctgaccta atgttggcca
3061 ttccacattg aggggacaag gtcagtgatg ccccccttca ctcacaagca cttcagaggc
3121 atgcagagag aagggacact cggccagctc tctgaggtaa tcagtgcaag gaggagtccg
3181 ttttttgcca gcaaacctca gcaggatcac actggaacag aacctggtca tacctgtgac
3241 aacacagctg tgagccaggg caaaccaccc actgtcactg gctcgagagt ctgggcagag
3301 gctctgaccc tccacccttt aaactggatg ccggggcctg gctgggccca atgccaagtg
3361 gttatggcaa ccctgactat ctggtcttaa catgtagctc aggaagtgga ggcgctaatg
3421 tccccaatcc ctggggattc ctgattccag ctattcatgt aagcagagcc aacctgccta
3481 tttctgtagg tgcgactggg atgttaggag cacagcaagg acccagctct gtagggctgg
3541 tgacctgata cttctcataa tggcatctag aagttaggct gagttggcct cactggccca
3601 gcaaaccaga acttgtcttt gtccgggcca tgttcttggg ctgtcttcta attccaaagg
3661 gttggttggt aaagctccac ccccttctcc tctgcctaaa gacatcacat gtgtatacac
3721 acacgggtgt atagatgagt taaaagaatg tcctcgctgg catcctaatt ttgtcttaag
3781 tttttttgga gggagaaagg aacaaggcaa gggaagatgt gtagctttgg ctttaaccag
3841 gcagcctggg ggctcccaag cctatggaac cctggtacaa agaagagaac agaagcgccc
3901 tgtgaggagt gggatttgtt tttctgtaga ccagatgaga aggaaacagg ccctgttttg
3961 tacatagttg caacttaaaa tttttggctt gcaaaatatt tttgtaataa agatttctgg
4021 gtaacaataa aaaaaaaaaa aaa
[SEQ ID NO: 20]
Translation = MEPAGPAPGRLGPLLLCLLLSASCECTGATGKELKVTQPERSVSVAAGDSTVLNCT
LTSLLPVGPIRWYRGVGPSRLLIYSFAGEYVPRIRNVSDTTKRNNMDESIRISNVTPADAGIYYCVKFQK
GSSEPDTEIQSGGGTEVYVLAKPSPPEVSGPADRGIPDQKVNFTCKSHGFSPRNITLKWFKDGQELHPLE
TTVNPSGKNVSYNISSTVRVVLNSMDVNSKVICEVAHITLDRSPLRGIANLSNFIRVSPTVKVTQQSPTS
MNQVNLTCRAERFYPEDLQLIWLENGNVSRNDTPKNLTKNTDGTYNYTSLFLVNSSAHREDVVFTCQVKH
DQQPAITRNHTVLGFAHSSDQGSMQTFPDNNATHNWNVFIGVGVACALLVVLLMAALYLLRIKQKKAKGS
TSSTRLHEPEKNAREITQVQSLIQDTNDINDITYADLNLPKEKKPAPRAPEPNNHTEYASIETGKVPRPE
DTLTYADLDMVHLSRAQPAPKPEPSFSEYASVQVQRK
LOCUS NM_001291020 3845 bp mRNA linear ROD 15 Feb. 2015
DEFINITION Mus musculus signal-regulatory protein alpha (Sirpa),
                        transcript variant 5, mRNA.
ACCESSION NM 001291020 XM 006498984
VERSION NM_001291020.1 GI: 597436945
KEYWORDS RefSeq.
SOURCE Mus musculus (house mouse)
[SEQ ID NO: 21]
      1 aagctcccct gccgcgggca gcctcttgcc cactggagtc taaggactgg ccgggtgaga
    61 ggccgagacc agggggcgat cggccgccac ttccccagtc caccttaaga ggaccaagta
  121 gccagcccgc cgcgccgacc tcagaaaaac aagtttgcgc aaagtggtgc gcggccagcc
  181 tctgggcaga gggagcggtg cttccaccgc ctggcagccc tgcgcgcggc ggcgcagccg
  241 cggcccatgg agcccgccgg cccggcccct ggccgcctag ggccgctgct gctctgcctg
  301 ctgctctccg cgtcctgttt ctgtacagga gccacgggga aggaactgaa ggtgactcag
  361 cctgagaaat cagtgtctgt tgctgctggg gattcgaccg ttctgaactg cactttgacc
  421 tccttcttgc cggtgggacc cattaggtgg tacagaggag tagggccaag ccggctgttg
  481 atctacagtt tcgcaggaga atacgttcct cgaattagaa atgtttcaga tactactaag
  541 agaaacaata tcgacttttc catccgtatc agtaatgtca ccccagcaga tgctggcatc
  601 tactactgtg tgaagttcca gaaaggatca tcagagcctg acacagaaat acaatctgga
  661 gggggaacag aggtctatgt actcgccaaa ccttctccac ccgaggtatc cggcccagca
  721 gacaggggca tacctgacca gaaagtgaac ttcacctgca agtctcatgg cttctctccc
  781 cggaatatca ccctgaagtg gttcaaagat gggcaagaac tccacccctt ggagaccacc
  841 gtgaacccta gtggaaagaa tgtctcctac aacatctcca gcacagtcag ggtggtacta
  901 aactccatgg atgttaattc taaggtcatc tgcgaggtag cccacatcac cttggataga
  961 agccctcttc gtcggattgc taacctgtct aacttcatcc gagtttcacc caccgtgaag
1021 gtcacccaac agtccccgac gtcaatgaac caggtgaacc tcacctgccg ggctgagagg
1081 ttctaccccg aggatctcca gctgatctgg ctggagaatg gaaacgtatc acggaatgac
1141 acgcccaaga atctcacaaa gaacacggat gggacctata attacacaag cttgttcctg
1201 gtgaactcat ctgctcatag agaggacgtg gtgttcacgt gccaggtgaa gcacgaccaa
1261 cagccagcga tcacccgaaa ccataccgtg ctgggatttg cccactcgag tgatcaaggg
1321 agcatgcaaa ccttccctga taataatgct acccacaact ggaatgtctt catcggtgtg
1381 ggcgtggcgt gtgctttgct cgtagtcctg ctgatggctg ctctctacct cctccggatc
1441 aaacagaaga aagccaaggg gtcaacatct tccacacggt tgcacgagcc cgagaagaac
1501 gccagggaaa taacccaggt acagtctttg atccaggaca caaatgacat caacgacatc
1561 acatacgcag acctgaatct gcccaaagag aagaagcccg caccccgggc ccctgagcct
1621 aacaaccaca cagaatatgc aagcattgag acaggcaaag tgcctaggcc agaggatacc
1681 ctcacctatg ctgacctgga catggtccac ctcagccggg cacagccagc ccccaagcct
1741 gagccatctt tctcagagta tgctagtgtc caggtccaga ggaagtgaat ggggctgtgg
1801 tctgtactag gccccatccc cacaagtttt cttgtcctac atggagtggc catgacgagg
1861 acatccagcc agccaatcct gtccccagaa ggccaggtgg cacgggtcct aggaccaggg
1921 gtaagggtgg cctttgtctt ccctccgtgg ctcttcaaca cctcttgggc acccacgtcc
1981 ccttcttccg gaggctaggt gttgcagaac cagagggcga actggagaaa gctgcctgga
2041 atccaagaag tcttgtgcct cggcccatca ctcgtgggtc tggatcctgg tcttggcaac
2101 cccaggttgc gtccttgatg ttccagagct tggtcttctg tgtggagaag agctcaccat
2161 ctctacccaa cttgagcttt gggaccagac tccctttaga tcaaaccgcc ccatctgtgg
2221 aagaactaca ccagaagtca gcaagttttc agccaacagt gctggcctcc ccacctccca
2281 ggctgactag ccctggggag aaggaaccct ctcctcctag accagcagag actccctggg
2341 catgttcagt gtggccccac ctcccttcca gtcccagctt gcttcctcca gctagcacta
2401 actcagcagc atcgctctgt ggacgcctgt aaattattga gaaatgtgaa ctgtgcagtc
2461 ttaaagctaa ggtgttagaa aatttgattt atgctgttta gttgttgttg ggtttctttt
2521 ctttttaatt tctttttctt ttttgatttt ttttctttcc cttaaaacaa cagcagcagc
2581 atcttcgctc tttgtcatgt gttgaatggt tgggtcttgt gaagtctgag gtctaacagc
2641 ttattgtcct ggaaggattt tcttacagca gaaacagatt tttttcaaat tcccagaatc
2701 ctgaggacca agaaggatcc ctcagctgct acttccagca cccagcgtca ctgggacgaa
2761 ccaggccctg ttcttacaag gccacatggc tggccctttg cctccatggc tactgtggta
2821 agtgcagcct tgtctgaccc aatgctgacc taatgttggc cattccacat tgaggggaca
2881 aggtcagtga tccccccctt cactcacaag cacttcagag gcatgcagag agaagggaca
2941 ctcggccagc tctctgaggt aatcagtgca aggaggagtc cgttttttgc cagcaaacct
3001 cagcaggatc acactggaac agaacctggt catacctgtg acaacacagc tgtgagccag
3061 ggcaaaccac ccactgtcac tggctcgaga gtctgggcag aggctctgac cctccaccct
3121 ttaaactgga tgccggggcc tggctgggcc caatgccaag tggttatggc aaccctgact
3181 atctggtctt aacatgtagc tcaggaagtg gaggcgctaa tgtccccaat ccctggggat
3241 tcctgattcc agctattcat gtaagcagag ccaacctgcc tatttctgta ggtgcgactg
3301 ggatcttagg agcacagcaa ggacccagct ctgtagggct ggtgacctga tacttctcat
3361 aatggcatct agaagttagg ctgagttggc ctcactggcc cagcaaacca gaacttgtct
3421 ttgtccgggc catgttcttg ggctgtcttc taattccaaa gggttggttg gtaaagctcc
3481 acccccttct cctctgccta aagacatcac atgtgtatac acacacgggt gtatagatga
3541 gttaaaagaa tgtcctcgct ggcatcctaa ttttgtctta agtttttttg gagggagaaa
3601 ggaacaaggc aagggaagat gcgtagcttt ggctttaacc aggcagcctg ggggctccca
3661 agcctatgga accctggtac aaagaagaga acagaagcgc cctgtgagga gtgggatttg
3721 tttttctgta gaccagatga gaaggaaaca ggccctgttt tgtacatagt tccaacttaa
3781 aatttttggc ttgcaaaata tttttgtaat aaagatttct gggtaacaat aaaaaaaaaa
3841 aaaaa
[SEQ ID NO: 20]
Translation = MEPAGPAPGRLGPLLLCLLLSASCFCTGATGKELKVTQPEKSVSVAAGDSTVLNCT
LTSLLPVGPIRWYRGVGPSRLLIYSFAGEYVPRIRNVSDTTKRNNMDESIRISNVTPADAGIYYCVKFQK
GSSEPDTEIQSGGGTEVYVLAKPSPPEVSGPADRGIPDQKVNFTCKSHGFSPRNITLKWFKDGQELHPLE
TTVNPSGKNVSYNISSTVRVVLNSMDVNSKVICEVAHITLDRSPLRGIANLSNFIRVSPTVKVTQQSPTS
MNQVNLTCRAERFYPEDLQLIWLENGNVSRNDTPKNLTKNTDGTYNYTSLFLVNSSAHREDVVETCQVKH
DQQPAITRNHTVLGFAHSSDQGSMQTFPDNNATHNWNVFIGVGVACALLVVLLMAALYLLRIKQKKAKGS
TSSTRLHEPEKNAREITQVQSLIQDTNDINDITYADLNLPKEKKPAPRAPEPNNHTEYASIETGKVPRPE
DTLTYADLDMVHLSRAQPAPKPEPSFSEYASVQVQRK
LOCUS NM_001291021 3389 bp mRNA linear ROD 15 Feb. 2015
DEFINITION Mus musculus signal-regulatory protein alpha (Sirpa),
                        Transcript variant 6, mRNA.
ACCESSION NM_001291021 XM_006498987
VERSION NM_001291021.1 GI: 597436920
SOURCE Mus musculus (house mouse)
[SEQ ID NO: 22]
      1 cgggaaggtg cgggcgcgag gagggggcgc tcggccgggc cgccctcgcg ctggcctcgc
    61 gacggctccg cacagcccgc actcgctctg cgagctgtcc ccgctcgcgc ttgctctccg
  121 atctccgtcc ccgctccctc tccctcttcc tctccccctc tttccttctc cctcgctatc
  181 cgctcccccg cccccgtgcc tctggctctg cgcctggctc cctcgggtcc gctccccttt
  241 cccgccggcc tggcccggcg tcacgctccc ggagtctccc cgctcggcgg cgtctcattg
  301 tgggaggggg tcagatcacc ccgccgggcg gtggcgctgg ggggcagcgg agggggaggg
  361 gccttagtcg ttcgcccgcg ccgcccgccc gcctgccgag cgcgctcacc gccgctctcc
  421 ctccttgctc tgcagccgcg gcccatggag cccgccggcc cggcccctgg ccgcctaggg
  481 ccgctgctgc tctgcctgct gctctccgcg tcctgtttct gtacaggagc cacggggaag
  541 gaactgaagg tgactcagcc tgagaaatca gtgtctgttg ctgctgggga ttcgaccgtt
  601 ctgaactgca ctttgacctc cttgttgccg gtgggaccca ttaggtggta cagaggagta
  661 gggccaagcc ggctgttgat ctacagtttc gcaggagaat acgttcctcg aattagaaat
  721 gtttcagata ctactaagag aaacaatatg gacttttcca tccgtatcag taatgtcacc
  781 ccagcagatg ctggcatcta ctactgtgtg aagttccaga aaggatcatc agagcctgac
  841 acagaaatac aatctggagg gggaacagag gtctatgtac tcgataataa tgctacccac
  901 aactggaatg tcttcatcgg tgtgggcgtg gcgtgtgctt tgctcgtagt cctgctgatg
  961 gctgctctct acctcctccg gatcaaacag aagaaagcca aggggtcaac atcttccaca
1021 cggttgcacg agcccgagaa gaacgccagg gaaataaccc aggtacagtc tttgatccag
1081 gacacaaatg acatcaacga catcacatac gcagacctga atctgcccaa agagaagaag
1141 cccgcacccc gggcccctga gcctaacaac cacacagaat atgcaagcat tgagacaggc
1201 aaagtgccta ggccagagga taccctcacc tatgctgacc tggacatggt ccacctcagc
1261 cgggcacagc cagcccccaa gcctgagcca tctttctcag agtatgctag tgtccaggtc
1321 cagaggaagt gaatggggct gtggtctgta ctaggcccca tccccacaag ttttcttgtc
1381 ctacatggag tcgccatgac gaggacatcc agccagccaa tcctgtcccc agaaggccag
1441 gtggcacggg tcctaggacc aggggtaagg gtggcctttg tcttccctcc gtggctcttc
1501 aacacctctt gggcacccac gtccccttct tccggaggct gggtgttgca gaaccagagg
1561 gcgaactgga gaaagctgcc tggaatccaa gaagtgttgt gcctcggccc atcactcgtg
1621 ggtctggatc ctggtcttgg caaccccagg ttgcgtcctt gatgttccag agcttggtct
1681 tctgtgtgga gaagagctca ccatctctac ccaacttgag ctttgggacc agactccctt
1741 tagatcaaac cgccccatct gtggaagaac tacaccagaa gtcagcaagt tttcagccaa
1801 cagtgctggc ctccccacct cccaggctga ctagccctgg ggagaaggaa ccctctcctc
1861 ctagaccagc agagactccc tgggcatgtt cagtgtggcc ccacctccct tccagtccca
1921 gcttgcttcc tccagctagc actaactcag cagcatcgct ctgtggacgc ctgtaaatta
1981 ttgagaaatg tcaactgtgc agtcttaaag ctaaggtgtt agaaaatttg atttatgctg
2041 tttagttgtt gttgggtttc ttttcttttt aatttctttt tcttttttga ttttttttct
2101 ttcccttaaa acaacagcag cagcatcttg gctctttgtc atgtgttgaa tggttgggtc
2161 ttgtgaagtc tgaggtctaa cagtttattg tcctggaagg attttcttac agcagaaaca
2221 gatttttttc aaattcccag aatcctgagg accaagaagg atccctcagc tcctacttcc
2281 agcacccagc gtcactggga cgaaccaggc cctgttctta caaggccaca tggctggccc
2341 tttgcctcca tcgctactgt ggtaagtgca gccttgtctg acccaatgct gacctaatgt
2401 tggccattcc acattgaggg gacaaggtca gtgatgcccc ccttcactca caagcacttc
2461 agaggcatgc agagagaagg gacactcggc cagctctctg aggtaatcag tgcaaggagg
2521 agtccgtttt ttgccagcaa acctcagcag gatcacactg gaacagaacc tggtcatacc
2581 tctgacaaca cagctgtgag ccagggcaaa ccacccactg tcactggctc gagagtctgg
2641 gcagaggctc tgaccctcca ccctttaaac tggatgccgg ggcctggctg ggcccaatgc
2701 caagtggtta tcgcaaccct gactatctgg tcttaacatg tagctcagga agtggaggcg
2761 ctaatgtccc caatccctgg ggattcctga ttccagctat tcatgtaagc agagccaacc
2821 tgcctatttc tctaggtgcg actgggatgt taggagcaca gcaaggaccc agctctgtag
2881 ggctggtgac ctgatacttc tcataatggc atctagaagt taggctgagt tggcctcact
2941 ggcccagcaa accagaactt gtctttgtcc gggccatgtt cttgggctgt cttctaattc
3001 caaagggttg gttggtaaag ctccaccccc ttctcctctg cctaaagaca tcacatgtgt
3061 atacacacac gggtgtatag atgagttaaa agaatgtcct cgctggcatc ctaattttgt
3121 cttaagtttt tttggaggga gaaaggaaca aggcaaggga agatgtgtag ctttggcttt
3181 aaccaggcag cctgggggct cccaagccta tggaaccctg gtacaaagaa gagaacagaa
3241 gcgccctgtg aggagtggga tttgtttttc tgtagaccag atgagaagga aacaggccct
3301 gttttgtaca tagttgcaac ttaaaatttt tcgcttgcaa aatatttttg taataaagat
3361 ttctgggtaa caataaaaaa aaaaaaaaa
[SEQ ID NO: 23]
Translation = MEPAGPAPGRLGPLLLCLLLSASCFCTGATGKELKVTQPEKSVSVAAGDSTVLNCT
LTSLLPVGPIRWYRGVGPSRLLIYSFAGEYVPRIRNVSDTTKRNNMDFSIRISNVTPADAGIYYCVKFQK
GSSEPDTEIQSGGGTEVYVLDNNATHNWNVFIGVGVACALLVVLLMAALYLLRIKQKKAKGSTSSTRLHE
PEKNAREITQVQSLIQDTNDINDITYADINLPKEKKPAPRAPEPNNHTEYASIETGKVPRPEDTLTYADL
DMVHLSRAQPAPKPEPSFSEYASVQVQRK
LOCUS NM_001291022 3020 bp mRNA linear ROD 15 Feb. 2015
DEFINITION Mus musculus signal-regulatory protein alpha (Sirpa),
                        Transcript variant 7, mRNA.
ACCESSION NM_001291022
VERSION NM_001291022.1 GI: 597436963
SOURCE Mus musculus (house mouse)
[SEQ ID NO: 24]
      1 cgggaaggtg cgggcgcgag gagggggcgc tcggccgggc cgccctcgcg ctggcctcgc
    61 gacggctccg cacagcccgc actcgctctg cgagctgtcc ccgctcgcgc ttgctctccg
  121 atctccgtcc ccgctccctc tccctcttcc tctccccctc tttccttctc cctcgctatc
  181 cgctcccccg cccccgtgcc tctggctctg cgcctggctc cctcgggtcc gctccccttt
  241 cccgccggcc tggcccggcg tcacgctccc ggagtctccc cgctcggcgg cgtctcattg
  301 tcggaggggg tcagatcacc ccgccgggcg gtggcgctgg ggggcagcgg agggggaggg
  361 gccttagtcg ttcgcccgcg ccgcccgccc gcctgccgag cgcgctcacc gccgctctcc
  421 ctccttgctc tccagccgcg gcccatggag cccgccggcc cggcccctgg ccgcctaggg
  481 ccgctgctgc tctgcctgct gctctccgcg tcctgtttct gtacagataa taatgctacc
  541 cacaactgga atgtcttcat cggtgtgggc gtggcgtgtg ctttgctcgt agtcctgctg
  601 atcgctgctc tctacctcct ccggatcaaa cagaagaaag ccaaggggtc aacatcttcc
  661 acacggttgc acgagcccga gaagaacgcc agggaaataa cccagatcca ggacacaaat
  721 gacatcaacg acatcacata cgcagacctg aatctgccca aagagaagaa gcccgcaccc
  781 cgcgcccctg agcctaacaa ccacacagaa tatgcaagca ttgagacagg caaagtgcct
  841 aggccagagg ataccctcac ctatgctgac ctggacatgg tccacctcag ccgggcacag
  901 ccagccccca agcctgagcc atctttctca gagtatgcta gtgtccaggt ccagaggaag
  961 tgaatggggc tgtggtctgt actaggcccc atccccacaa gttttcttgt cctacatgga
1021 gtggccatga cgaggacatc cagccagcca atcctgtccc cagaaggcca ggtggcacgg
1081 gtcctaggac caggggtaag ggtggccttt gtcttccctc cgtggctctt caacacctct
1141 tcggcaccca cgtccccttc ttccggaggc tcggtgttgc agaaccagag ggcgaactgg
1201 agaaagctgc ctggaatcca agaagtgttg tgcctcggcc catcactcgt gggtctggat
1261 cctggtcttg graaccccag gttgcgtcct tgatgttcca gagcttggtc ttctgtgtgg
1321 agaagagctc accatctcta cccaacttga gctttgggac cagactccct ttagatcaaa
1381 ccgccccatc tctggaagaa ctacaccaga agtcagcaag ttttcagcca acagtgctgg
1441 cctccccacc tcccaggctg actagccctg gggagaagga accctctcct cctagaccag
1501 cagagactcc ctgggcatgt tcagtgtggc cccacctccc ttccagtccc agcttgcttc
1561 ctccagctag cactaactca gcagcatcgc tctgtggacg cctgtaaatt attgagaaat
1621 gtgaactgtg cagtcttaaa gctaaggtgt tagaaaattt gatttatgct gtttagttgt
1681 tgttgggttt cttttctttt taatttcttt ttcttttttg attttttttc tttcccttaa
1741 aacaacagca gcagcatctt ggctctttgt catgtgttga atggttgggt cttgtgaagt
1801 ctgaggtcta acagtttatt gtcctggaag gattttctta cagcagaaac agattttttt
1861 caaattccca gaatcctgag gaccaagaag gatccctcag ctgctacttc cagcacccag
1921 cgtcactggg acgaaccagg ccctgttctt acaaggccac atggctggcc ctttgcctcc
1981 atggctactg tcgtaagtgc agccttgtct gacccaatgc tgacctaatg ttggccattc
2041 cacattgagg ggacaaggtc agtgatgccc cccttcactc acaagcactt cagaggcatg
2101 cagagagaag ggacactcgg ccagctctct gaggtaatca gtgcaaggag gagtccgttt
2161 tttgccagca aacctcagca ggatcacact ggaacagaac ctggtcatac ctgtgacaac
2221 acagctgtga gccagggcaa accacccact gtcactggct cgagagtctg ggcagaggct
2281 ctgaccctcc accctttaaa ctggatgccg gggcctggct gggcccaatg ccaagtggtt
2341 atggcaaccc tgactatctg gtcttaacat gtagctcagg aagtggaggc gctaatgtcc
2401 ccaatccctg gggattcctg attccagcta ttcatgtaag cagagccaac ctgcctattt
2461 ctgtaggtgc gactgggatg ttaggagcac agcaaggacc cagctctgta gggctggtga
2521 cctgatactt ctcataatgg catctagaag ttaggctgag ttggcctcac tggcccagca
2581 aaccagaact tgtctttgtc cgggccatgt tcttgggctg tcttctaatt ccaaagggtt
2641 ggttggtaaa gctccacccc cttctcctct gcctaaagac atcacatgtg tatacacaca
2701 cgggtgtata gatgagttaa aagaatgtcc tcgctggcat cctaattttg tcttaagttt
2761 ttttggaggg agaaaggaac aaggcaaggg aagatgtgta gctttggctt taaccaggca
2821 gcctgggggc tcccaagcct atggaaccct ggtacaaaga agagaacaga agcgccctgt
2881 gaggagtggg atttgttttt ctgtagacca gatgagaagg aaacaggccc tgttttgtac
2941 atagttgcaa cttaaaattt ttggcttgca aaatattttt gtaataaaga tttctgggta
3001 acaataaaaa aaaaaaaaaa
[SEQ ID NO: 25]
Translation = MEPAGPAPGRLGPLLLCLLLSASCFCTDNNATHNWNVFIGVGVACALLVVLLMAAL
YLLRIKQKKAKGSTSSTRLHEPEKNAREITQIQDTNDINDITYADLNLPKEKKPAPRAPEPNNHTEYASI
ETGKVPRPEDTLTYADLDMVHLSRAQPAPKPEPSFSEYASVQVQRK
LOCUS NM_009020 3393 bp mRNA linear ROD 15 Feb. 2015
DEFINITION Mus musculus recombination activating gene 2 (Rag2), mRNA.
ACCESSION NM_009020
VERSION NM_009020.3 GI: 144227233
SOURCE Mus musculus (house mouse)
[SEQ ID NO: 26]
      1 actctaccct gcagccttca gcttggcaca aactaaacag tgactcttcc ccaagtgccg
    61 agtttaattc ctggcttggc cgaaaggatt cagagaggga taagcagccc ctctggcctt
  121 cagtcccaaa ataagaaaga gtatttcaca tccacaagca ggaagtacac ttcatacctc
  181 tctaagataa aagacctatt cacaatcaaa aatgtccctg cagatggtaa cagtgggtca
  241 taacatagcc ttaattcaac caggcttctc acttatgaat tttgatggcc aagttttctt
  301 ctttcgccag aaaggctggc ctaagagatc ctgtcctact ggagtctttc attttgatat
  361 aaaacaaaat catctcaaac tgaagcctgc aatcttctct aaagattcct gctacctccc
  421 acctcttcgt tatccagcta cttgctcata caaaggcagc atagactctg acaagcatca
  481 atatatcatt cacggaggga aaacaccaaa caatgagctt tccgataaga tttatatcat
  541 gtctgtcgct tccaagaata acaaaaaagt tactttccgt tgcacagaga aagacttagt
  601 aggagatgtc cctgaaccca gatacggcca ttccattgac gtggtgtata gtcgagggaa
  661 aagcatgggt gttctctttg gaggacgttc atacatgcct tctacccaga gaaccacaga
  721 aaaatggaat agtgtagctg actgcctacc ccatgttttc ttgatagatt ttgaatttgg
  781 gtgtgctaca tcatatattc tcccagaact tcaggatggg ctgtcttttc atctttctat
  841 tcccagaaac gataccgttt atattttggg aggacactca cttgccagta atatacgccc
  901 tcctaacttg tatagaataa gagtggacct tcccctgggt accccagcag tgaattgcac
  961 agtcttgcca ggaggaatct ctgtctccag tccaatcctc actcaaacaa acaatgatga
1021 atttcttatt gtgggtggtt atcagctgga aaatcagaaa aggatggtct gcagccttgt
1081 ctctctaggg gacaacacga ttgaaatcag tgagatggag actcctgact ggacctcaga
1141 tattaagcat agcaaaatat gctttggaag caacatggga aacgggacta ttttccttgg
1201 cataccagga gacaataagc aggctatgtc agaagcattc tatttctata ctttgagatg
1261 ctctgaagag gatttgagtg aagatcagaa aattgtctcc aacagtcaga catcaacaga
1321 agatcctggg gactccactc cctttgaaga ctcagaggaa ttttgtttca gtgctgaagc
1381 aaccagtttt gatggtgacg atgaatttga cacctacaat gaagatgatg aagatgacga
1441 gtctgtaacc ggctactgga taacatgttg ccctacttgt gatgttgaca tcaatacctg
1501 ggttccgttc tattcaacgg agctcaataa acccgccatg atctattgtt ctcatgggga
1561 tcggcactgg gtacatgccc agtgcatgga tttggaagaa cgcacactca tccacttgtc
1621 agaaggaagc aacaagtatt attgcaatga acatgtacag atagcaagag cattgcaaac
1681 tcccaaaaga aaccccccct tacaaaaacc tccaatgaaa tccctccaca aaaaaggctc
1741 tcggaaagtc ttgactcctg ccaagaaatc cttccttaga agactgtttg attaatttag
1801 caaaagcccc tcagactcag gtatattgct ctctgaatct actttcaatc ataaacatta
1861 ttttgatttt tgtttactga aatctctatg ttatgtttta gttatgtgaa ttaagtgctg
1921 ttctgattta ttgttaagta taactattct aatgtgtgtt ttttaacatc ttatccagga
1981 atgtcttaaa tgagaaatgt tatacagttt tccattaagg atatcagtga taaagtatag
2041 aactcttaca ttattttgta acaatctaca tattgaatag taactaaata ccaataaata
2101 aactaatgca caaaaagtta agttcttttg tgtaataagt agcctatagt tggtttaaac
2161 agttaaaacc aacagctata tcccacacta ctgctgttta taaattttaa ggtggcctct
2221 ggtttatact tatgagcaga attatatata ttggtcaata ccatgaagaa aaatttaatt
2281 ctatatcaag ccaggcatgg tgatggtgat acatgcctgt aatcctggca cttaggaagt
2341 ggaagaagga agtttgtgag tttgatgctt gttgaggtat gaccttttgc tatgtattgt
2401 agtgtatgag ccccaagacc tgcttgaccc agagacaaga gagtccacac atagatccaa
2461 gtaatgctat gtgaccttgc cccccggtta cttgtgatta ggtgaataaa gatgtcaaca
2521 gccaatagct gggcagaaga gccaaaagtg gggattgagg gtaccctggc ttgatgtagg
2581 aggagaccat gaggaaaggg gagaaaaaag tgatggagga ggagaaagat gccatgagct
2641 aggagttaag aaagcatggc catgagtgct ggccaattgg agttaagagc agcccagatg
2701 aaacatagta agtaataact cagggttatc gatagaaaat agattttagt gccgtactct
2761 ccccagccct agagctgact atggcttact gtaaatataa agtttgtatg tctcttttat
2821 ccaggaacta aatggtcaaa ggtggagtag aaactctgga ttgggattaa atttttctac
2881 aacaaatgct ggcctgggct agattttatc tcatatccga aggctgacag aacacagagc
2941 actcgtaaca ttgccacctg ccatgcacaa agacctgagt ctaatactgt ggacattttc
3001 ttgaagtatc tacatgtact tctggagtga aaacatattc caacaatatg cctttgttta
3061 aatcactcac tcactttggg ccctcacatt atatcctttc aaaatcaatg gttcacccct
3121 ttcaaaatgc ttagccatag tccctcatct tccttaaaga cagttgtcat ctctggaaat
3181 agtcacatgt cattcaaggt ccaatactgt gcagctctga agtatggcat taccacttta
3241 agtgaaaagt gaaatatgaa catgagctca gacaaaggtt tcggactatc actctcaagg
3301 aggctctact gctaagtcct gaactgcttt cacatgaata cagaaattat aacaaaaaat
3361 atgtaatcaa taaaaagaaa actttcatat tcc
[SEQ ID NO: 27]
Translation = MSLQMVTVGHNIALIQPGFSLMNFDGQVFFFGQKGWPKRSCPTGVFHFDIKQNHLK
LKPAIFSKDSCYLPPLRYPATCSYKGSIDSDKHQYIIHGGKTPNNELSDKIYIMSVACKNNKKVTFRCTE
KDLVGDVPEPRYGHSIDVVYSRGKSMGVLFGGRSYMPSTQRTTEKWNSVADCLPHVFLIDFEFGCATSYI
LPELQDGLSFHVSIARNDTVYILGGHSLASNIRPANLYRIRVDLPLGTPAVNCTVLPGGISVSSAILTQT
NNDEFVIVGGYQLENQKRMVCSLVSLGDNTIEISEMETPDWTSDIKHSKIWEGSNMGNGTIFLGIPGDNK
QAMSEAFYFYTLRCSEEDLSEDQKIVSNSQTSTEDPGDSTPFEDSEEFCFSAEATSFDGDDEFDTYNEDD
EDDESVTGYWITCCPTCDVDINTWVPFYSTELNKPAMIYCSHGDGHWVHAQCMDLEERTLIHLSEGSNKY
YCNEHVQIARALQTPKRNPPLQKPPMKSLHKKGSGKVITPAKKSFLRRLFD
LOCUS NM_013563 1612 bp mRNA linear ROD 15 Feb. 2015
DEFINITION Mus musculus interleukin 2 receptor, gamma
                        chain (Il2rg), mRNA.
ACCESSION NM_013563
VERSION NM_013563.3 GI: 118129799
SOURCE Mus musculus (house mouse)
[SEQ ID NO: 28]
      1 gacacagact acacccagag aaagaagagc aagcaccatg ttgaaactat tattgtcacc
    61 tagatccttc ttagtccttc agctgctcct gctgagggca gggtggagct ccaaggtcct
  121 catgtccagt gcgaatgaag acatcaaagc tgatttgatc ctgacttcta cagcccctga
  181 acacctcagt gctcctactc tgccccttcc agaggttcag tgctttgtgt tcaacataga
  241 gtacatgaat tgcacttgga atagcagttc tgagcctcag gcaaccaacc tcacgctgca
  301 ctataggtac aaggtatctg ataataatac attccaggag tgcagtcact atttgttctc
  361 caaagagatt acttctggct gtcagataca aaaagaagat atccagctct accagacatt
  421 tgttgtccag ctccaggacc cccagaaacc ccagaggcga gctgtacaga agctaaacct
  481 acagaatctt gtgatcccac gggctccaga aaatctaaca ctcagcaatc tgagtgaatc
  541 ccagctagag ctgagatgga aaagcagaca tattaaagaa cgctgtttac aatacttggt
  601 gcagtaccgg agcaacagag atcgaagctg gacggaacta atagtgaatc atgaacctag
  661 attctccctg cctagtgtgg atgagctgaa acggtacaca tttcgggttc ggagccgcta
  721 taacccaatc tgtggaagtt ctcaacagtg gagtaaatgg agccagcctg tccactgggg
  781 gagtcatact gtagaggaga atccttcctt gtttgcactg gaagctgtgc ttatccctgt
  841 tggcaccatg gggttgatta ttaccctgat ctttgtgtac tgttggttgg aacgaatgcc
  901 tccaattccc cccatcaaga atctagagga tctggttact gaataccaag ggaacttttc
  961 ggcctggagt ggtgtgtcta aagggctgac tgagagtctg cagccagact acagtgaacg
1021 gttctgccac gtcagcgaga ttccccccaa aggaggggcc ctaggagagg ggcctggagg
1081 ttctccttgc agcctgcata gcccttactg gcctccccca tcttattctc tgaagccgga
1141 agcctgaaca tcaatccttt gatggaacct caaagtccta tagtcctaag tgacgctaac
1201 ctcccctact caccttggca atctggatcc aatgctcact gccttccctt ggggctaagt
1261 ttcgatttcc tgtcccatgt aactgctttt ctgttccata tgccctactt gagagtgtcc
1321 cttgccctct ttccctgcac aagccctccc atgcccagcc taacaccttt ccactttctt
1381 tgaagagagt cttaccctgt agcccagggt ggctgggagc tractatgta ggccaggttg
1441 gcctccaact cacaggctat cctcccacct ctgcctcata agagttgggg ttactggcat
1501 gcaccaccac acccagcatg gtccttctct tttataggat tctccctccc tttttctacc
1561 tatgattcaa ctgtttccaa atcaacaaga aataaagttt ttaaccaatg at
[SEQ ID NO: 29]
Translation = MLKLLLSPRSELVLQLLLLRAGWSSKVLMSSANEDIKADLILTSTAPEHLSAPTLP
LPEVQCFVENIEYMNCTWNSSSEPQATNLTLHYRYKVSDNNTFQECSHYLESKEITSGCQIQKEDIQLYQ
TFVVQLQDPQKPQRRAVQKLNLQNLVIPRAPENLTLSNLSESQLELRWKSRHIKERCLQYLVQYRSNRDR
SWTELIVNHEPRFSLPSVDELKRYTFRVRSRYNPICGSSQQWSKWSQPVHWGSHTVEENPSLFALEAVLI
PVGTMGLIITLIFVYCWLERMPPIPPIKNLEDLVTEYQGNESAWSGVSKGLTESLQPDYSERFCHVSEIP
PKGGALGEGPGGSPCSLHSPYWPPPCYSLKPEA
LOCUS NM_000585 2012 bp mRNA linear PRI 15 Mar. 2015
DEFINITION Homo sapiens interleukin 15 (IL15), transcript
                        variant 3, mRNA.
ACCESSION NM_000585
VERSION NM_000585.4 GI: 323098327
SOURCE Homo sapiens (human)
[SEQ ID NO: 30]
      1 gttgggactc cgggtggcag gcgcccgggg gaatcccagc tgactcgctc actgccttcg
    61 aagtccggcg ccccccggga gggaactggg tggccgcacc ctcccggctg cggtggctgt
  121 cgccccccac cctgcagcca ggactcgatg gagaatccat tccaatatat ggccatgtgg
  181 ctctttcgag caatgttcca tcatgttcca tgctgctgac gtcacatgga gcacagaaat
  241 caatcttagc agatagccag cccatacaag atcgtattgt attgtaggag gcattgtgga
  301 tcgatggctg ctggaaaccc cttgccatag ccagctcttc ttcaatactt aaggatttac
  361 cgtcgctttg agtaatgaga atttcgaaac cacatttgag aagtatttcc atccagtgct
  421 acttctgttt acttctaaac agtcattttc taactgaagc tggcattcat gtcttcattt
  481 tgggctgttt cagtgcaggg cttcctaaaa cagaagccaa ctgggtgaat gtaataagtg
  541 atttgaaaaa aattgaagat cttattcaat ctatgcatat tgatgctact ttatatacgg
  601 aaagtgatgt tcaccccagt tccaaagtaa cagcaatgaa gtgctttctc ttggagttac
  661 aagttatttc acttgagtcc ggagatgcaa gtattcatga tacagtagaa aatctgatca
  721 tcctagcaaa caacagtttg tcttctaatg ggaatgtaac agaatctgga tccaaagaat
  781 gtgaggaact ggaggaaaaa aatattaaag aatttttgca gagttttgta catattgtcc
  841 aaatgttcat caacacttct tgattgcaat tgattctttt taaagtgttt ctgttattaa
  901 caaacatcac tctgctgctt agacataaca aaacactcgg catttcaaat gtgctgtcaa
  961 aacaagtttt tctgtcaaga agatgatcag accttggatc agatgaactc ttagaaatga
1021 aggcagaaaa atgtcattga gtaatatagt gactatgaac ttctctcaga cttactttac
1081 tcattttttt aatttattat tgaaattgta catatttgtg gaataatgta aaatcttgaa
1141 taaaaatatg tacaagtgtt gttttttaag ttgcactgat attttacctc ttattgcaaa
1201 atagcatttg tttaagggtg atagtcaaat tatgtattgg tggggctggg taccaatgct
1261 gcaggtcaac agctatgctg gtaggctcct gccagtgtgg aaccactgac tactggctct
1321 cattgacttc cttactaagc atagcaaaca gaggaagaat ttgttatcag taagaaaaag
1381 aagaactata tctgaatcct cttctttata ctgtaattta gttattgatg tataaagcaa
1441 ctgttatgaa ataaagaaat tccaataact ggcatataat gtccatcagt aaatcttggt
1501 ggtggtggca ataataaact tctactgata gctagaatgg tgtgcaagct tgtccaatca
1561 cggattgcag gccacatgcg gcccaggaca actttgaatg tcgcccaaca caaattcata
1621 aactttcata catctcgttt ttagctcatc agctatcatt agcggtagtg tatttaaagt
1681 gtcgcccaag acaattcttc ttattccaat gtggcccagg gaaatcaaaa gattggatgc
1741 ccctggtata gaaaactaat agtgacagtg ttcatatttc atgctttccc aaatacaggt
1801 attttatttt cacattcttt ttgccatgtt tatataataa taaagaaaaa ccctgttgat
1861 ttgttggagc cattgttatc tgacagaaaa taattgttta tattttttgc actacactgt
1921 ctaaaattag caagctctct tctaatggaa ctgtaagaaa gatgaaatat ttttgtttta
1981 ttataaattt atttcacctt aaaaaaaaaa aa
[SEQ ID NO: 31]
Translation = MRISKPHLRSISIQCYLCLLLNSHFLTEAGIHVFILGCFSAGLPKTEANWVNVISD
LKKIEDLIQSMHIDATLYTESDVHPSCKVTAMKCFLLELQVISLESGDASIHDTVENLIILANNSLSSNG
NVTESGCKECEELEEKNIKEFLQSFVHIVQMFINTS
LOCUS NM_172175 2333 bp mRNA linear PRI 15 Mar. 2015
DEFINITION Homo sapiens interleukin 15 (IL15), transcript
                        variant 2, mRNA.
ACCESSION NM_172175
VERSION NM_172175.2 GI: 323098328
SOURCE Homo sapiens (human)
[SEQ ID NO: 32]
      1 gttgggactc cgggtggcag gcgcccgggg gaatcccagc tgactcgctc actgccttcg
    61 aagtccggcg ccccccggga gggaactggg tggccgcacc ctcccggctg cggtggctgt
  121 cgccccccac cctgcagcca ggactcgatg gagaatccat tccaatatat ggccatgtgg
  181 ctctttggag caatgttcca tcatgttcca tgctgctgac gtcacatgga gcacagaaat
  241 caatgttagc agatagccag cccatacaag atcgttttca actagtggcc ccactgtgtc
  301 cggaattgat gggttcttgg tctcactgac ttcaagaatg aagccgcgga ccctcgcggt
  361 gagtcttaca gctcttaagg tggcgcatct ggagtttgtt ccttctgatg ttcggatgtg
  421 ttcggagttt cttccttctg gtgggttcgt ggtctcgctg gctcaggagt gaagctacag
  481 accttcgcgg aggcattgtg gatggatggc tcctggaaac cccttgccat agccagctct
  541 tcttcaatac ttaaggattt accgtggctt tgagtaatga gaatttcgaa accacatttc
  601 agaagtattt ccatccagtg ctacttgtgt ttacttctaa acagtcattt tctaactgaa
  661 gctggcattc atgtcttcat tttgggatgc agctaatata cccagttggc ccaaagcacc
  721 taacctatag ttatataatc tgactctcag ttcagtttta ctctactaat gccttcatgg
  781 tattgggaac catagatttg tgcagctgtt tcagtgcagg gcttcctaaa acagaagcca
  841 actgggtgaa tgtaataagt gatttgaaaa aaattgaaga tcttattcaa tctatgcata
  901 ttgatgctac tttatatacg gaaagtgatg ttcaccccag ttgcaaagta acagcaatga
  961 agtgctttct cttggagtta caagttattt cacttgagtc cggagatgca agtattcatg
1021 atacagtaga aaatctgatc atcctagcaa acaacagttt gtcttctaat gggaatgtaa
1081 cagaatctgg atgcaaagaa tgtgaggaac tcgaggaaaa aaatattaaa gaatttttgc
1141 agagttttgt acatattgtc caaatgttca tcaacacttc ttgattgcaa ttgattcttt
1201 ttaaagtgtt tctgttatta acasacatca ctctgctgct tagacataac aaaacactcg
1261 gcatttcaaa tctgctgtca aaacaagttt ttctgtcaag aagatgatca gaccttggat
1321 cagatgaact cttagaaatg aaggcagaaa aatgtcattg agtaatatag tgactatgaa
1381 cttctctcag acttacttta ctcatttttt taatttatta ttgaaattgt acatatttgt
1441 ggaataatgt aaaatgttga ataaaaatat gtacaagtgt tcttttttaa gttgcactga
1501 tattttacct cttattgcaa aatagcattt gtttaagggt gatagtcaaa ttatgtattg
1561 gtggggctgg gtaccaatgc tccaggtcaa cagctatgct ggtaggctcc tcccagtgtg
1621 gaaccactga ctactggctc tcattgactt ccttactaag catagcaaac agaggaagaa
1681 tttcttatca gtaagaaaaa gaagaactat atgtgaatcc tcttctttat actgtaattt
1741 agttattgat gtataaagca actgttatga aataaagaaa ttgcaataac tcgcatataa
1801 tgtccatcag taaatcttgg tggtggtggc aataataaac ttctactgat aggtagaatg
1861 gtgtgcaagc ttgtccaatc acggattgca ggccacatgc ggcccaggac aactttgaat
1921 gtcgcccaac acaaattcat aaactttcat acatctcgtt tttagctcat cagctatcat
1981 tagcggtagt gtatttaaag tgtggcccaa gacaattctt cttattccaa tgtggcccag
2041 ggaaatcaaa agattggatg cccctggtat agaaaactaa tagtgacagt gttcatattt
2101 catgctttcc caaatacagg tattttattt tcacattctt tttgccatgt ttatataata
2161 ataaagaaaa accctgttga tttgttggag ccattgttat ctgacagaaa ataattgttt
2221 atattttttg cactacactg tctaaaatta graagctctc ttctaatgga actgtaagaa
2281 agatgaaata tttttgtttt attataaatt tatttcacct taaaaaaaaa aaa
[SEQ ID NO: 33]
Translation = MVLGTIDLCSCFSAGLPKTEANWVNVISDLKKIEDLIQSMHIDATLYTESDVHPSC
KVTAMKCFLLELQVISLESGDASIHDTVENLIILANNSLSSNGNVTESGCKECEELEEKNIKEFLQSFVH
IVQMFINTS
LOCUS NM_008357 1297 bp mRNA linear ROD 15 Feb. 2015
DEFINITION Mus musculus interleukin 15 (Il15), transcript
                        variant 1, mRNA.
ACCESSION NM_008357
VERSION NM_008357.2 GI: 363000959
SOURCE Mus musculus (house mouse)
[SEQ ID NO: 34]
      1 ttcttgacca agacttcaat actcagtggc actgtattcc ccttctgtcc agccactctt
    61 ccccagagtt ctcttcttca tcctccccct tccagagtag ggcagcttgc aggtcctcct
  121 gcaagtctct cccaattctc tgcgcccaaa agacttgcag tgcatctcct tacgcgctgc
  181 agggaccttg ccagggcagg actgcccccg cccagttgca gagttggacg aagacgggat
  241 cctgctgtgt ttggaaggct gagttccaca tctaacagct cagagaggtc aggaaagaat
  301 ccaccttgac acatggccct ctggctcttc aaagcactgc ctcttcatgg tccttgctgg
  361 tgaggtcctt aagaacacag aaacccatgt cagcagataa ccagcctaca ggaggccaag
  421 aagagttctg gatggatggc agctggaagc ccatcgccat agccagctca tcttcaacat
  481 tgaagctctt acctgggcat taagtaatga aaattttgaa accatatatg aggaatacat
  541 ccatctcgtg ctacttgtgt ttccttctaa acagtcactt tttaactgag gctggcattc
  601 atgtcttcat tttgggctgt gtcagtgtag gtctccctaa aacagaggcc aactggatag
  661 atgtaagata tgacctggag aaaattgaaa gccttattca atctattcat attgacacca
  721 ctttatacac tgacagtgac tttcatccca gttgcaaagt tactgcaatg aactgctttc
  781 tcctggaatt gcaggttatt ttacatgagt acagtaacat gactcttaat gaaacagtaa
  841 gaaacgtgct ctaccttgca aacagcactc tctcttctaa caagaatgta gcagaatctg
  901 gctgcaagga atgtgaggag ctggaggaga aaaccttcac agagtttttg caaagcttta
  961 tacgcattgt ccaaatgttc atcaacacgt cctgactgca tgcgagcctc ttccgtgttt
1021 ctgttattaa ggtacctcca cctgctgctc agaggcagca cagctccatg catttgaaat
1081 ctgctgggca aactaagctt cctaacaagg agataatgag ccacttggat cacatgaaat
1141 cttggaaatg aagagaggaa aagagctcgt ctcagactta tttttgcttg cttattttta
1201 atttattgct tcatttgtac atatttgtaa tataacagaa gatgtggaat aaagttgtat
1261 ggatatttta tcaattgaaa tttaaaaaaa aaaaaaa
[SEQ ID NO: 35]
Translation = MKILKPYMENTSISCYLCFLLNSHELTEAGIHVFILGCVSVGLPKTEANWIDVRYD
LEKIESLIQSIHIDTTLYTDSDFHPSCKVTAMNCFLLELQVILHEYSNMTLNETVRNVLYLANSTLSSNK
NVAESGCKECEELEEKTETEFLQSFIRIVQMFINTS
LOCUS NM_001254747 1287 bp mRNA linear ROD 15 Feb. 2015
DEFINITION Mus musculus interleukin 15 (I115), transcript
                        variant 2, mRNA.
ACCESSION NM_001254747
VERSION NM_001254747.1 GI: 363000983
SOURCE Mus musculus (house mouse)
[SEQ ID NO: 36]
      1 ttcttgacca agacttcaat actcagtggc actgtattcc ccttctgtcc agccactctt
    61 ccccagagtt ctcttcttca tcctccccct tccagagtag ggcagcttgc aggtcctcct
  121 gcaagtctct cccaattctc tgcgcccaaa agacttgcag tgcatctcct tacgcgctgc
  181 agggaccttg ccagggcagg actgcccccg cccagttgca gagttggacg aagacgggat
  241 cctgctgtgt ttggaaggct gagttccaca tctaacagct cagagagaat ccaccttgac
  301 acatggccct ctggctcttc aaagcactgc ctcttcatgg tccttgctgg tgaggtcctt
  361 aagaacacag aaacccatgt cagcagataa ccagcctaca ggaggccaag aagagttctg
  421 gatggatggc agctggaagc ccatcgccat agccagctca tcttcaacat tgaagctctt
  481 acctgggcat taagtaatga aaattttgaa accatatatg aggaatacat ccatctcgtg
  541 ctacttgtgt ttccttctaa acagtcactt tttaactgag gctggcattc atgtcttcat
  601 tttgggctgt gtcagtgtag gtctccctaa aacagaggcc aactggatag atgtaagata
  661 tgacctggag aaaattgaaa gccttattca atctattcat attgacacca ctttatacac
  721 tcacagtgac tttcatccca gttgcaaagt tactgcaatg aactgctttc tcctggaatt
  781 gcaggttatt ttacatgagt acagtaacat gactcttaat gaaacagtaa gaaacgtgct
  841 ctaccttgca aacagcactc tgtcttctaa caagaatgta gcagaatctg gctgcaagga
  901 atgtgaggag ctggaggaga aaaccttcac agagtttttg caaagcttta tacgcattgt
  961 ccaaatgttc atcaacacgt cctgactgca tgcgagcctc ttccgtgttt ctgttattaa
1021 ggtacctcca cctgctgctc agaggcagca cagctccatg catttgaaat ctgctgggca
1081 aactaagctt cctaacaagg agataatgag ccacttggat cacatgaaat cttggaaatg
1141 aagagaggaa aagagctcgt ctcagactta tttttgcttg cttattttta atttattgct
1201 tcatttgtac atatttgtaa tataacagaa gatgtggaat aaagttgtat ggatatttta
1261 tcaattgaaa tttaaaaaaa aaaaaaa
[SEQ ID NO: 35]
Translation = MKILKPYMENTSISCYLCFLLNSHFLTEAGIHVFILGCVSVGLPKTEANWIDVRYD
LEKIESLIQSIHIDTTLYTDSDFHPSCKVTAMNCFLLELQVILHEYSNMTLNETVRNVLYLANSTLSSNK
NVAESGCKECEELEEKTFTEFLQSFIRIVQMFINTS
```

1.-67. (canceled)

68. A rodent embryonic stem (ES) cell comprising in its genome:
a signal-regulatory protein alpha (SIRPα) gene comprising a nucleic acid sequence that encodes a signal-regulatory protein alpha (Sirpa) protein operably linked to an endogenous rodent SIRPα gene promoter at the rodent SIRPα gene locus in the genome of the ES cell, wherein the SIRPα gene comprises a replacement of a portion of an endogenous rodent SIRPα coding sequence, and wherein the encoded SIRPα protein comprises a human SIRPα extracellular domain, possesses wild-type human SIRPα receptor function, and comprises a signaling domain of an endogenous rodent SIRPα protein; and
an interleukin-15 (IL-15) gene comprising a nucleic acid sequence that encodes an IL-15 (IL-15) protein, wherein the nucleic acid sequence includes exons 5-8 of a human IL-15 gene, wherein the nucleic acid sequence is operably linked to an endogenous rodent IL-15 gene promoter at the rodent IL-15 gene locus in the genome of the ES cell, wherein the IL-15 gene comprises a replacement of some or all of an endogenous rodent IL-15 coding sequence, and wherein the encoded IL-15 protein possesses wild-type human IL-15 signaling function.

69. The rodent ES cell of claim 68, wherein the SIRPα gene comprises exon 1 of a rodent SIRPα gene, exons 2, 3 and 4 of a human SIRPα gene, and exons 5, 6, 7 and 8 of the rodent SIRPα gene.

70. The rodent ES cell of claim 68, wherein the rodent ES cell is homozygous for the SIRPα gene.

71. The rodent ES cell of claim 68, wherein the IL-15 gene comprises exons 3 and 4 of a rodent IL-15 gene.

72. The rodent ES cell of claim 68, wherein the rodent ES cell is homozygous for the IL-15 gene.

73. The rodent ES cell of claim 68, wherein:
the SIRPα gene comprises a replacement of exons 2, 3 and 4 of the rodent SIRPα gene at the endogenous rodent SIRPα locus with exons 2, 3 and 4 of the human SIRPα gene;
the IL-15 gene comprises a replacement of exons 5, 6, 7 and 8 of the rodent IL-15 gene at the endogenous rodent IL-15 locus with exons 5, 6, 7 and 8 of the human IL-15 gene.

74. The rodent ES cell of claim 73, wherein:
wherein the SIRPα gene comprises exon 1 of a rodent SIRPα gene, exons 2, 3 and 4 of a human SIRPα gene, and exons 5, 6, 7 and 8 of the rodent SIRPα gene;
wherein the IL-15 gene comprises exons 3 and 4 of a rodent IL-15 gene;
wherein the rodent ES cell is homozygous for the SIRPα gene; and
wherein the rodent ES cell is homozygous for the IL-15 gene.

75. The rodent ES cell of claim 68, wherein the rodent ES cell comprises a Rag2 gene knock-out.

76. The rodent ES cell of claim 68, wherein the rodent ES cell comprises an IL2rg gene knock-out.

77. The rodent ES cell of claim 68, wherein the rodent ES cell comprises a Rag2 gene knock-out and an IL2rg gene knock-out.

78. The rodent ES cell of claim 74, wherein the rodent ES cell comprises a Rag2 gene knock-out and an IL2rg gene knock-out.

79. The rodent ES cell of claim 68, wherein the rodent ES cell is a mouse ES cell.

80. A rodent embryo comprising the rodent ES cell of claim 68.

81. A mouse embryo comprising the mouse ES cell of claim 79.

82. A method of making a genetically modified rodent, the method comprising making a genetically modified rodent from the rodent embryo of claim 80.

83. A method of making a genetically modified mouse, the method comprising making a genetically modified mouse from the mouse embryo of claim 81.
